(19) United States
(12) Patent Application Publication
(10) Pub. No.: US 2011/0293745 A1
(43) Pub. Date: Dec. 1, 2011

(54) AURORA KINASE INHIBITORS

(75) Inventors: Ute Hoch, San Francisco, CA (US); Jeffrey A. Silverman, Burlingame, CA (US); Daniel C. Adelman, Redwood City, CA (US)

(73) Assignee: SUNESIS PHARMACEUTICALS, INC., South San Francisco, CA (US)

(21) Appl. No.: 12/922,487

(22) PCT Filed: Mar. 16, 2009
(86) PCT No.: PCT/US09/37292

§ 371 Date: Aug. 19, 2011

Related U.S. Application Data
(60) Provisional application No. 61/036,817, filed on Mar. 14, 2008.
(60) Provisional application No. 61/053,658, filed on May 15, 2008.
(60) Provisional application No. 61/045,583, filed on Apr. 16, 2008.

(51) Int. Cl. A61K 31/519; A61K 31/704; A61P 35/02; A61K 31/7068; C07D 495/04; A61P 35/00

(52) U.S. Cl. 424/649; 544/278; 514/260.1; 514/49; 514/34

(57) ABSTRACT

The present invention provides Compound 1, solid forms thereof, compositions thereof, as Aurora kinase inhibitor for use as an oncology agent. The present invention also provides synthetic methods of preparing Compound 1 and intermediates thereto.

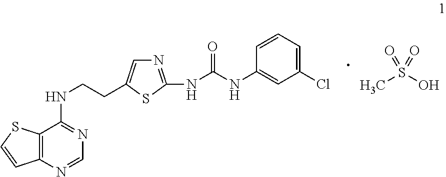

### CROSS-REFERENCE TO RELATED APPLICATIONS

The present invention claims priority to U.S. provisional application Ser. No. 61/036,817, filed Mar. 14, 2008, U.S. provisional application Ser. No. 61/045,583, filed Apr. 16, 2008, and U.S. provisional application Ser. No. 61/053,658, filed May 15, 2008, the entirety of each of which is hereby incorporated herein by reference.

### BACKGROUND OF THE INVENTION

Aurora kinases constitute a family of serine-threonine kinases; members of the family are referred to herein collectively as Aurora kinase. Aurora kinase upregulation and/or amplification has been strongly associated with cancer. For example, Aurora kinase overexpression and/or amplification has been observed in cervical cancer, ovarian cancer, and neuroblastoma cell lines [Warner, S. L. et al., Molecular Cancer Therapeutics 2:589-95 (2003)]. Furthermore, Aurora kinase overexpression and/or amplification has been observed also in primary clinical isolates of cancers. Additionally, higher expression levels of Aurora kinase(s) have been associated with increased levels of aggressiveness in certain cancer types.

On a cellular level, Aurora kinases play crucial roles in mitotic cell division, both in ensuring accurate division of genomic material in the nucleus and also in division of cytoplasm (cytokinesis). Disruption of activity of the Aurora kinases leads to multiple mitotic defects including aberrant centrosome duplication, misalignment of chromosomes, inhibition of cytokinesis, and disruption of the spindle checkpoint. These defects in mitosis result in cells having abnormal counts of chromosomes (aneuploidy) and programmed cell death (apoptosis).

There are three mammalian Aurora gene products: Aurora A, Aurora B and Aurora C. Aurora A and B are essential in mitosis. The role of Aurora C is unclear; however, Aurora C can complement Aurora B kinase activity in mitosis.

Elevated expression of Aurora A transcripts and/or protein has been detected in a high percentage of colon, breast, ovarian, gastric, pancreatic, bladder and liver tumors, and the AURKA chromosome locus (20q13) is amplified in a subset of these tumors. Aurora A mRNA overexpression has also been reported to be associated with proliferative activity in mantle cell lymphoma (MCL) and non-Hodgkin's lymphoma (NHL). Aurora B transcripts and/or protein have been found to be expressed at a high level in cancers of the thyroid, lung, prostate, endometrium, brain, and mouth, and in colorectal cancers. Aurora C is also expressed at high levels in primary tumors. Thus, there remains a need for developing a small-molecule antagonist of Aurora kinase activity as an oncology agent.

### SUMMARY OF THE INVENTION

It has now been found that Compound 1:

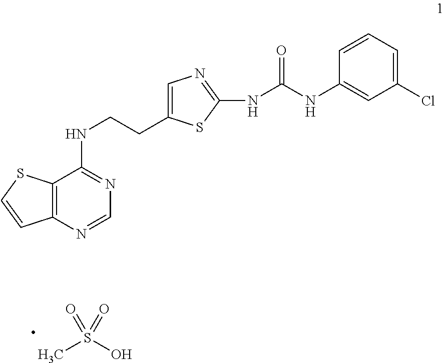

is particularly useful as an Aurora kinase (“Aurora”) inhibitor and is therefore useful for treating disorders mediated by Aurora. Also provided herein are, among other things, solid forms of Compound 1, pharmaceutical compositions which comprise Compound 1, methods for making Compound 1 and intermediates thereof, and methods of using the same in the treatment of Aurora-mediated disorders. Such embodiments and others are described herein.

### BRIEF DESCRIPTION OF THE DRAWINGS

FIG. 1A shows the inhibition of Aurora A; and FIG. 1B shows the inhibition of Aurora B enzymatic activity in vitro by Compound 1, as measured by a homogeneous time-resolved fluorescence assay.

FIG. 2 shows a detail of co-crystal obtained of Aurora-A with Compound 2. The protein is depicted in ribbon form except for the DFG motif region (labeled on the lower right), which is shown as a Van der Waals surface. Compound 2 (center), is also depicted as a Van der Waals surface.

FIG. 3 shows HCT 116 cells exposed to DMSO vehicle (depicted in black line, black fill) or to 36 nM Compound 1 (depicted in gray line, white fill) for 16 hours. Cells were stained with propidium iodide and subjected to cell sorting. Cell count is plotted against total cell fluorescence.

FIG. 4 shows HCT 116 cells exposed to DMSO vehicle or to 16 nM Compound 1 for 72 hours, followed by staining for DNA (with propidium iodide) and tubulin (with anti-tubulin antibody).

FIG. 5 shows the dose-dependent effect in the amount of phosphohistone H3 in HCT 116 cells upon exposure to Compound 1, as measured by High Content Screening.

FIGS. 6A and 6B depicts the concentration of Compound 2 in tumor (black circles) and in plasma (gray diamonds) over time in HCT 116 tumor xenograft mice after IP administration of a 170 mg/kg dose of the compound. T₁₁₂ of Compound 2 in tumor and in plasma are also depicted in this Figure.

FIG. 7 shows Western blots of phosphorylation of histone H3 in HCT 116 tumor xenograft mice after IP administration of vehicle, 50 mg/kg of Compound 1, or 100 mg/kg Compound 1. Concentrations of Compound 1 in the tumor are shown below the blots. Blots are shown for 3 hours, 6 hours, and 10 hours after administration of the compound.

FIG. 8 depicts representative Caspase-3 (upper row) and hematoxylin and eosin (H&E) (lower row) sections prepared from tumors after completion of treatment with 170 mg/kg Compound 1 on a bi-weekly schedule for 3 consecutive weeks. Treatments were administered on Day 1, 4, 8, 11, 15, and 18, with tumors being excised Day 4, 11, 18, and 25 of study. All images were taken at 40× magnification.

FIG. 9 shows tumor volume (mm³) at various times after implantation for HCT 116 colon cancer xenograft mice treated with vehicle (inverted triangles); treated with 125 mg/kg Compound 1 once a week for three weeks (squares); 150 mg/kg Compound 1 twice a week for three weeks (triangles); or 100 mg/kg per day, two times, with an interval of 9 days off between the two treatments.

FIG. 10A shows pharmacokinetics of Compound 2 over time after intravenous administration in mouse (squares), rat (diamonds) and dog (circles). FIG. 10B shows pharmacokinetics of Compound 2 in mice after intraperitoneal (IP), intravenous (IV), and oral (PO) administration. The routes of administration are depicted respectively as circles, squares, and triangles.

FIG. 11A shows exposure of Compound 2 by female mice (squares), female dog (solid triangles), male dogs (open triangles), female rats (solid circles), and male rats (open circles) as a function of the dose of Compound 1 administered. FIG. 11B shows the AUClast, as defined herein, for female rats (solid squares), and male rats (open squares).

FIG. 12A shows the mean percentage recovery of Compound 2 in rats over time in the following elimination pathways: bile (squares), feces (diamonds), and urine (triangles). FIG. 12B shows amounts of radioactively-labeled Compound 2 and metabolites thereof as measured in rat bile. FIG. 12C shows a map of the distribution of metabolites observed in samples of plasma, bile and urine from treated rats.

FIG. 13 shows a hypothetical example of measurement of drug cooperation. FIG. 13A depicts effect of cooperation on EC₅₀ (effective concentration) curves; FIG. 13B shows effect of cooperation on Cl₅₀ (combination index) data. FIG. 13C shows representative results for the hypothetical combinations.

FIG. 14A shows High Content Screening (HCS) cell count data for Compound 1 as combined with various drugs in wild type (shown in black) and p53−/− cells (shown in gray) HCT 116 colon cancer cells. Compound 1 was dosed first, and the combination drug was dosed second. Compound 1 dosed in combination with itself is depicted in open symbols; Compound 1 dosed with a different drug is shown in solid symbols. High/Low, High/High, and Low/High ratios of Compound 1 to combination drugs were used, as shown left to right. FIG. 14B shows data from Compound 1 administered with other drugs: (i) as a co-dose; (ii) with Compound 1 administered prior to the combination drug; and (iii) with the combination drug administered prior to Compound 1. Results for High/Low, High/High, and Low/High ratios of Compound 1 are shown left to right. In addition, results are shown in the presence or in the absence of p53 RNAi.

FIG. 15A shows results of a CellTiter Blue® cell proliferation assay using Compound 1 in combination with itself (open symbols) or a combination drug (solid symbols) in HCT 116 colon cancer cells. High/High and Low/High ratios of Compound 1 to combination drug are shown left to right. FIG. 15B shows quantitative results for the experiment, including statistical significance.

FIG. 16 shows DNA morphologies of HCT 116 cells treated with (top row, left to right) DMSO vehicle, docetaxel, and vincristine, respectively; and with (bottom row, left to right) Compound 1, Compound 1 and docetaxel, and Compound 1 and vincristine, respectively. Large arrows and small arrow indicate DNA morphologies of polyploidy and condensed chromatin, respectively.

FIG. 17 shows an HCT 116 mouse xenograft study. Mice were treated according to schedules presented schematically at the top of this Figure and described further herein, with vehicle (open squares); 10 mg/kg docetaxel administered as a single agent (solid circles); 42.5 mg/kg Compound 1 administered as a single agent (open circles); 10 mg/kg docetaxel administered prior to 42.5 mg/kg Compound 1 (inverted open triangles); and 42.5 mg/kg Compound 1 administered prior to 10 mg/kg docetaxel (open triangles).

FIG. 18 depicts an XRPD pattern obtained for Form A of Compound 1.

FIG. 19 depicts the DSC pattern obtained for Form A of Compound 1.

FIG. 20 depicts an XRPD pattern obtained for Form B of Compound 1.

FIG. 21 depicts the DSC pattern obtained for Form B of Compound 1.

FIG. 22 depicts an XRPD pattern obtained for Form C of Compound 1.

FIG. 23 depicts the DSC pattern obtained for Form C of Compound 1.

FIG. 24 depicts an XRPD pattern obtained for Form D of Compound 1.

FIG. 25 depicts the DSC pattern obtained for Form D of Compound 1.

FIG. 26 depicts an XRPD pattern obtained for Form E of Compound 1.

FIG. 27 depicts the DSC pattern obtained for Form E of Compound 1.

FIG. 28 depicts an XRPD pattern obtained for Form F of Compound 1.

FIG. 29 depicts the DSC pattern obtained for Form F of Compound 1.

FIG. 30 depicts an XRPD pattern obtained for Form G of Compound 1.

FIG. 31 depicts the DSC pattern obtained for Form G of Compound 1.

FIG. 32 depicts an XRPD pattern obtained for Form H of Compound 1.

FIG. 33 depicts the DSC pattern obtained for Form H of Compound 1.

FIG. 34 depicts an XRPD pattern obtained for Form I of Compound 1.

FIG. 35 depicts the DSC pattern obtained for Form I of Compound 1.

FIG. 36 depicts an XRPD pattern obtained for Form J of Compound 1.

FIG. 37 depicts the DSC pattern obtained for Form J of Compound 1.

FIG. 38 depicts an XRPD pattern obtained for Form K of Compound 1.

FIG. 39 depicts the DSC pattern obtained for Form K of Compound 1.

FIG. 40 depicts an XRPD pattern obtained for Form L of Compound 1.

FIG. 41 depicts the DSC pattern obtained for Form L of Compound 1.

FIG. 42 depicts photomicrographs of cells from HCT 116 xenograft mice treated with (top row) vehicle and (bottom row) Compound 1. A) shows epidermis (left) 4 days after treatment and (right) 18 days after treatment. B) shows bone marrow (left) eleven days after treatment and (right) eighteen days after treatment.

FIG. 43 shows correlation of plasma concentrations of Compound 1 with inhibition of phospho-histone H3 (pHH3) in tumor as measured in HCT 116 xenograft mice. A) A plot of (left y-axis and squares) plasma concentration of Compound 2 (μM) and (right y-axis and triangles) pHH3 levels one hour after administration against dose of Compound 1 administered. B) A plot of plasma concentration of Compound 2 plotted directly against pHH3 levels in U/mL one hour after administration of Compound 1. C) Western blots showing pHH3 and Histone H3 (HH3) levels after administration of Compound 1: (top blot, left to right) vehicle, 1 mg/kg, 2 mg/kg, 5 mg/kg to three HCT 116 mice for each dose; (bottom blot, left to right) vehicle, 10 mg/kg, and 20 mg/kg to three HCT 116 xenograft mice for each dose. D) Western blots showing pHH3 and HH3 levels, 6 hours, 9 hours, and 24 hours after administration of a single 170 mg/kg dose of Compound 1.

FIG. 44 shows induction of apoptosis in xenograft tumors after a single dose of Compound 1. A) Western blot showing cleaved PARP levels (as compared with β-actin control) for tumors from HCT 116 xenograft mice at 3 hours, 6 hours, and 12 hours after treatment with an IP dose of 170 mg/kg of Compound 1; three mice were treated at each dose. B) Western blot showing cleaved PARP and HH3 levels 2 hours, 6 hours and 24 hours after treatment of MV-4-11 xenograft mice with an IP dose of 50 mg/kg or 100 mg/kg of Compound 1; three mice were treated at each dose.

FIG. 45 shows observed form conversion from slurries and characterization of the various crystal forms observed.

### DETAILED DESCRIPTION OF THE INVENTION

### (1) Compound 1 and Compound 2

According to one embodiment, the present invention provides a mesylate salt of 1-(3-chlorophenyl)-3-{5-[2-(thieno[3,2-d]pyrimidin-4-ylamino)ethyl]thiazo}-urea, referred to herein as “Compound 1”:

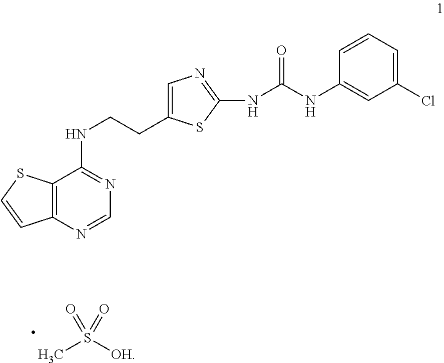

It has now been found that Compound 1, including compositions thereof, is particularly useful for treating disorders mediated by Aurora kinases. Compound 1 of the present invention is a novel small molecule that shows potent inhibition of Aurora kinases.

It will be appreciated by one of ordinary skill in the art that 1-(3-chlorophenyl)-3-{5-[2-(thieno[3,2-d]pyrimidin-4-ylamino)ethyl]thiazol-2-yl}-urea referred to herein as Compound 2:

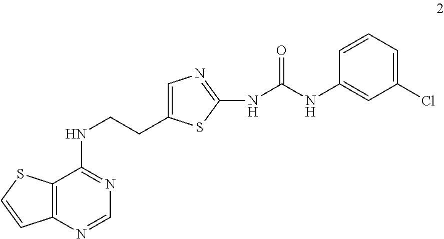

and methanesulfonic acid are ionically bonded to form Compound 1, i.e., the mesylate salt of Compound 2. Compound 2 is in the class of molecules described in US 2006/0035908 and WO 2006/036266, each of which is incorporated herein by reference for all that they disclose.

It is contemplated that Compound 1 can be provided in a variety of physical forms. For example, Compound 1 can be put into solution, suspension, or be provided in solid form. When Compound 1 is in solid form, said compound may be amorphous, crystalline, or a mixture thereof. Such solid forms are described in more detail below. Dosage amounts used in the compositions and methods provided herein are calculated based on Compound 2 (free base) rather than any particular salt form, even if it is the salt form itself that is used. For example, if a 750 mg/m² of Compound 1 is specified, the amount as used herein corresponds to the amount of Compound 1 that provides 750 mg/m² of the free base.

In general, Compound 1, and pharmaceutically acceptable compositions thereof, are useful as inhibitors (e.g., of Aurora kinases), and for the treatment of Aurora-mediated diseases or disorders including, but not limited to, cancers (e.g., bladder cancer, brain cancer, breast cancer, cervical cancer, colon cancer, esophageal cancer, head and neck cancer, leukemia, liver cancer, lung cancer, lymphoma, melanoma, myeloma, neuroendocrine cancer (e.g., neuroblastoma), ovarian cancer, pancreatic cancer, prostate cancer, renal cancer, sarcoma, skin cancer, stomach cancer, testicular cancer, thyroid cancer and uterine cancer); and hematological tumors (e.g., mantle cell lymphoma (MCL), Non-Hodgkin's lymphoma (NHL), Hodgkin's disease, acute myelogenous leukemia (AML), chronic myelogenous leukemia (CML), chronic lymphocytic leukemia (CLL) or acute lymphoblastic lymphoma (ALL)).

### DEFINITIONS

As used herein, the term “about”, when used in reference to any degree 2-theta value recited herein, refers to the stated value±0.1 degree 2-theta.

As used herein, the term “anhydrous” refers to a form of a compound that is substantially free of water. It has been found that Compound 1 can exist as an anhydrous and nonsolvated crystalline form, referred to herein as Form A. As used herein, the term “substantially free of water” means that no significant amount of water is present. For example, in certain embodiments when the term “substantially free of water” is applied herein to a solid form, it means that water content in the crystalline structure is less than 0.5% of the weight of the solid. In some embodiments of the invention, the term “substantially free of water” means that the water content is less than 1% of the weight of the solid. One of ordinary skill in the art will appreciate that an anhydrous solid can contain various amounts of residual water wherein that water is not incorporated in the crystalline lattice. Such incorporation of residual water can depend upon the compound's hygroscopicity and storage conditions.

The term “carrier” refers to any chemical compound moiety consistent with the stability of Compound 1. In certain embodiments, the term “carrier” refers to a pharmaceutically acceptable carrier. An exemplary carrier herein is water.

The expression “dosage form” refers to means by which a formulation is stored and/or administered to a subject. For example, the formulation may be stored in a vial or syringe. The formulation may also be stored in a container which protects the formulation from light (e.g., UV light). Alternatively, a container or vial which itself is not necessarily protective from light may be stored in a secondary storage container (e.g., an outer box, bag, etc.) which protects the formulation from light.

The term “formulation” refers to a composition that includes at least one pharmaceutically active compound (e.g., at least Compound 1) in combination with one or more excipients or other pharmaceutical additives for administration to a patient. In general, particular excipients and/or other pharmaceutical additives are typically selected with the aim of enabling a desired stability, release, distribution and/or activity of active compound(s) for applications.

The term “patient”, as used herein, means a mammal to which a formulation or composition comprising a formulation is administered, and includes humans.

As used herein, the term “polymorph” refers to different crystal structures achieved by a particular chemical entity. Specifically, polymorphs occur when a particular chemical compound can crystallize with more than one structural arrangement.

As used herein, the term “solvate” refers to a crystal form where a stoichiometric or non-stoichiometric amount of solvent, or mixture of solvents, is incorporated into the crystal structure. Similarly, the term “hydrate” refers to a crystal form where a stoichiometric or non-stoichiometric amount of water is incorporated into the crystal structure.

As used herein, the term “substantially all” when used to describe X-ray powder diffraction (“XRPD”) peaks of a compound means that the XRPD of that compound includes at least about 80% of the peaks when compared to a reference. For example, when an XRPD of a compound is said to include “substantially all” of the peaks in a reference list, or all of the peaks in a reference XRPD, it means that the XRPD of that compound includes at least 80% of the peaks in the specified reference. In other embodiments, the phrase “substantially all” means that the XRPD of that compound includes at least about 85, 90, 95, 97, 98, or 99% of the peaks when compared to a reference. Additionally, one skilled in the art will appreciate throughout, that XRPD peak intensities and relative intensities as listed herein may change with varying particle size and other relevant variables.

The term “substantially free of” when used herein in the context of a physical form of Compound 1 means that at least about 95% by weight of Compound 1 is in the specified solid form. In certain embodiments of the invention, the term “substantially free of” one or more other forms of Compound 1 means that at least about 97%, 98%, or 99% by weight of Compound 1 is in the specified solid form. For example, “substantially free of amorphous Compound 1” means that at least about 95% by weight of Compound 1 is crystalline. In certain embodiments of the invention, “substantially free of amorphous Compound 1” means that at least about 97%, 98%, or 99% by weight of Compound 1 is crystalline.

The term “substantially similar,” when used herein in the context of comparing X-ray powder diffraction or differential scanning calorimetry spectra obtained for a physical form of Compound 1, means that two spectra share defining characteristics sufficient to differentiate them from a spectrum obtained for a different form of Compound 1. In certain embodiments, the term “substantially similar” means that two spectra are the same.

As used herein, and unless otherwise specified, the terms “therapeutically effective amount” and “effective amount” of a compound refer to an amount sufficient to provide a therapeutic benefit in the treatment, prevention and/or management of a disease, to delay or minimize one or more symptoms associated with the disease or disorder to be treated. The terms “therapeutically effective amount” and “effective amount” can encompass an amount that improves overall therapy, reduces or avoids symptoms or causes of disease or disorder or enhances the therapeutic efficacy of another therapeutic agent.

The terms “treat” or “treating,” as used herein, refer to partially or completely alleviating, inhibiting, delaying onset of, reducing the incidence of, ameliorating and/or relieving a disorder or condition, or one or more symptoms of the disorder, disease or condition.

The expression “unit dose” as used herein refers to a physically discrete unit of a formulation appropriate for a subject to be treated. It will be understood, however, that the total daily usage of a formulation of the present invention will be decided by the attending physician within the scope of sound medical judgment. The specific effective dose level for any particular subject or organism will depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### (2) Solid Forms of Compound 1

It would be desirable to provide a solid form of Compound 1 that imparts characteristics such as improved aqueous solubility, stability and ease of formulation. In particular, such solid form may be thermodynamically stable in humid environments. Additionally, such solid form may be stable at relative humidities below 90% and be readily isolated as a free-flowing solid.

It has been found that Compound 1 can exist in a variety of solid forms. Such forms include anhydrous, non-solvated, hydrated, and solvated forms. Such solid forms include crystalline and amorphous forms. In some embodiments, Compound 1 is an anhydrous and non-solvated crystalline form. All such solid forms of Compound 1 are contemplated under the present invention. In certain embodiments, the present invention provides Compound 1 as a mixture of one or more solid forms selected from crystalline and amorphous.

In certain embodiments of the present invention, Compound 1 is provided as a crystalline solid. In certain embodiments, Compound 1 is a crystalline solid substantially free of amorphous Compound 1.

In certain embodiments, the present invention provides Compound 1 as an anhydrous and non-solvated crystalline form. In some embodiments, such an anhydrous and non-solvated crystalline form is Form A. In certain embodiments, the present invention provides Form A of Compound 1 substantially free of other solid forms of Compound 1.

In some embodiments, the present invention provides Form A of Compound 1 characterized in that it has one or more, two or more, or three or more, peaks in its XRPD pattern selected from those at about 8.5, 13.2, 15.3, 15.6, 16.7, 20.2, 20.6, 25.2, 26.4 and 27.0 degrees 2-theta. In certain embodiments, the present invention provides Form A of Compound 1, substantially free of other forms of Compound 1.

In other embodiments, Form A of Compound 1 is characterized in that is has substantially all of the peaks in its XRPD pattern listed in Table 1, below.

TABLE 1
XRPD Peaks Form A
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 8.4678 | 10.43367 | 6 | 0.17720 | 269 | 3458 |
| 2 | 10.3045 | 8.57770 | 1 | 0.20910 | 59 | 806 |
| 3 | 12.6558 | 6.98886 | 3 | 0.21560 | 105 | 1358 |
| 4 | 13.0200 | 6.79417 | 3 | 0.19200 | 110 | 1221 |
| 5 | 13.2243 | 6.68966 | 10 | 0.21350 | 423 | 4400 |
| 6 | 15.0400 | 5.88589 | 1 | 0.13000 | 49 | 453 |
| 7 | 15.3000 | 5.78645 | 7 | 0.24080 | 292 | 3415 |
| 8 | 15.5800 | 5.68308 | 7 | 0.20680 | 301 | 3143 |
| 9 | 15.8580 | 5.58407 | 6 | 0.21600 | 233 | 2689 |
| 10 | 16.4400 | 5.38768 | 3 | 0.22940 | 111 | 1533 |
| 11 | 16.7269 | 5.29591 | 8 | 0.22900 | 333 | 3787 |
| 12 | 17.7667 | 4.98824 | 4 | 0.22350 | 183 | 2366 |
| 13 | 19.0584 | 4.65297 | 4 | 0.21900 | 163 | 2010 |
| 14 | 19.5709 | 4.53226 | 5 | 0.21010 | 222 | 2521 |
| 15 | 20.1577 | 4.40163 | 8 | 0.31790 | 349 | 5877 |
| 16 | 20.6461 | 4.29860 | 21 | 0.29980 | 863 | 13866 |
| 17 | 22.6000 | 3.93118 | 1 | 0.33340 | 56 | 1093 |
| 18 | 22.8000 | 3.89715 | 2 | 0.20760 | 65 | 646 |
| 19 | 23.6583 | 3.75767 | 2 | 0.19670 | 95 | 1139 |
| 20 | 23.8908 | 3.72163 | 2 | 0.07270 | 80 | 298 |
| 21 | 24.1800 | 3.67776 | 1 | 0.12440 | 55 | 862 |
| 22 | 24.4200 | 3.64216 | 2 | 0.00000 | 68 | 0 |
| 23 | 24.6200 | 3.61302 | 2 | 0.00000 | 65 | 0 |
| 24 | 25.1659 | 3.53587 | 100 | 0.19490 | 4195 | 46845 |
| 25 | 25.4800 | 3.49299 | 2 | 0.10600 | 95 | 1219 |
| 26 | 25.9867 | 3.42602 | 2 | 0.18410 | 94 | 1038 |
| 27 | 26.4191 | 3.37092 | 7 | 0.21350 | 280 | 3137 |
| 28 | 27.0447 | 3.29435 | 7 | 0.22670 | 308 | 3871 |
| 29 | 28.6015 | 3.11848 | 4 | 0.27500 | 188 | 2892 |
| 30 | 30.1203 | 2.96460 | 6 | 0.29820 | 252 | 4411 |
| 31 | 30.5800 | 2.92107 | 1 | 0.24000 | 49 | 714 |
| 32 | 31.2458 | 2.86033 | 1 | 0.27830 | 44 | 658 |
| 33 | 33.1618 | 2.69931 | 1 | 0.19640 | 59 | 839 |
| 34 | 33.6641 | 2.66018 | 2 | 0.15970 | 66 | 653 |
| 35 | 34.2166 | 2.61848 | 2 | 0.32670 | 73 | 1158 |
| 36 | 34.7600 | 2.57878 | 2 | 0.13540 | 78 | 679 |
| 37 | 35.0796 | 2.55601 | 4 | 0.21920 | 147 | 2167 |
| 38 | 36.3556 | 2.46917 | 2 | 0.28370 | 97 | 1650 |
| 39 | 37.1494 | 2.41821 | 2 | 0.20290 | 63 | 766 |
| 40 | 37.8009 | 2.37802 | 2 | 0.16180 | 71 | 745 |
| 41 | 39.9821 | 2.25317 | 1 | 0.29570 | 54 | 1198 |
| 42 | 40.6153 | 2.21950 | 2 | 0.20600 | 102 | 1258 |
| 43 | 41.4789 | 2.37526 | 2 | 0.28220 | 82 | 1373 |
| 44 | 42.0600 | 2.14654 | 1 | 0.28000 | 60 | 1184 |
| 45 | 43.4996 | 2.07878 | 2 | 0.24730 | 87 | 1250 |

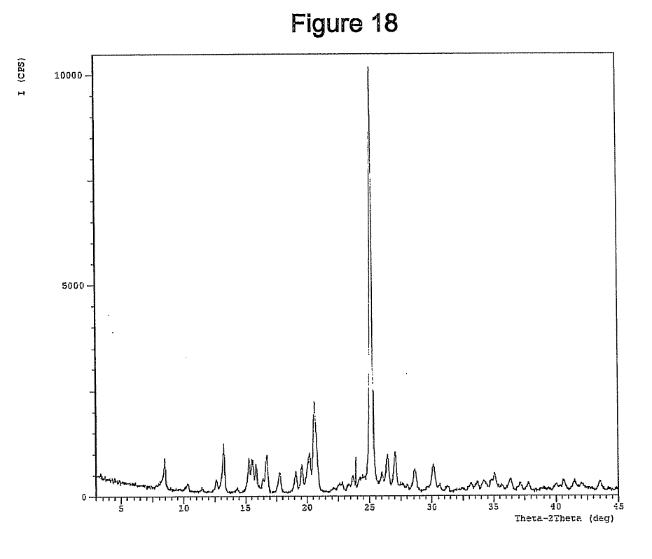

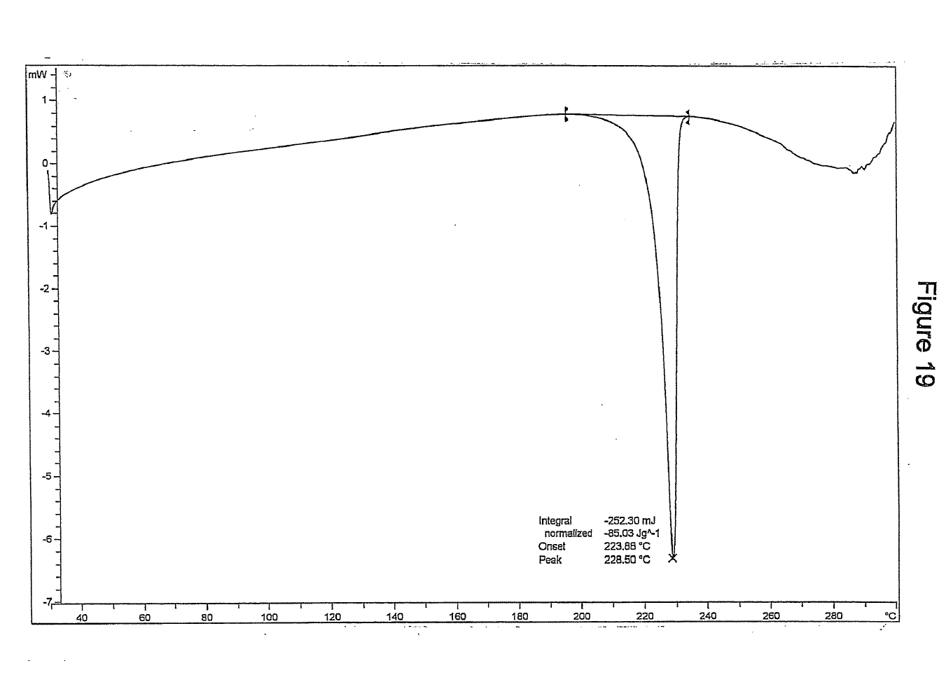

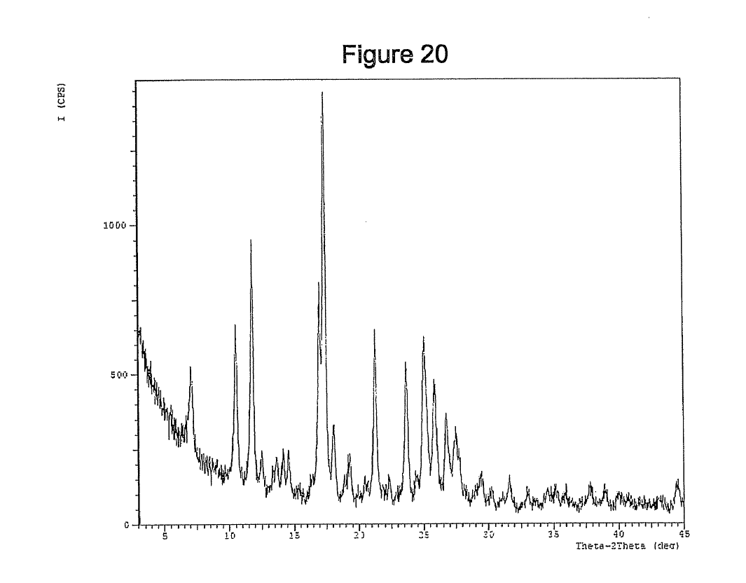

In some embodiments, the present invention provides Form A of Compound 1, having an X-ray diffraction pattern substantially similar to that depicted in FIG. 18. In one aspect, the present invention provides Form A having a DSC pattern substantially similar to that depicted in FIG. 19.

In certain embodiments, Compound 1 exists in at least one hydrate form. One such hydrate, i.e., as a monohydrate, is referred to herein as Form B. In certain embodiments, the present invention provides Form B of Compound 1. In some embodiments, the present invention provides Form B of Compound 1 characterized in that it has one or more, two or more, or three or more, peaks in its XRPD pattern selected from those at about 7.1, 10.5, 11.8, 17.0, 17.4, 18.0, 21.3, 23.7, 25.1, 25.8, 26.8, 27.4, and 27.7 degrees 2-theta. In certain embodiments, the present invention provides Form B of Compound 1, substantially free of other forms of Compound 1. In certain embodiments, Form B is monohydrate solid form of Compound 1.

In other embodiments, Form B of Compound 1 is characterized in that it has substantially all of the peaks in its XRPD pattern listed in Table 2, below.

TABLE 2
XRPD Peaks Form B
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 6.6600 | 13.26120 | 4 | 0.16660 | 19 | 317 |
| 2 | 7.0543 | 12.52082 | 19 | 0.30860 | 102 | 1662 |
| 3 | 10.2400 | 8.63159 | 8 | 0.14860 | 42 | 463 |
| 4 | 10.5261 | 8.39761 | 34 | 0.27570 | 184 | 2654 |
| 5 | 11.4000 | 7.75576 | 4 | 0.14400 | 22 | 293 |
| 6 | 11.7888 | 7.50083 | 55 | 0.26760 | 294 | 4360 |
| 7 | 12.5150 | 7.06717 | 8 | 0.25000 | 43 | 681 |
| 8 | 13.3800 | 6.61217 | 5 | 0.19200 | 25 | 281 |
| 9 | 13.6200 | 6.49619 | 8 | 0.32000 | 41 | 584 |
| 10 | 14.1464 | 6.25562 | 9 | 0.24710 | 47 | 647 |
| 11 | 14.5789 | 6.07100 | 9 | 0.23210 | 48 | 634 |
| 12 | 16.4200 | 5.39419 | 5 | 0.53000 | 28 | 1214 |
| 13 | 16.9800 | 5.21753 | 47 | 0.27520 | 251 | 3455 |
| 14 | 17.3667 | 5.10222 | 100 | 0.27830 | 535 | 7775 |
| 15 | 18.0424 | 4.91263 | 16 | 0.27910 | 84 | 1425 |
| 16 | 18.8600 | 4.70147 | 4 | 0.18000 | 20 | 213 |
| 17 | 19.2450 | 4.60827 | 9 | 0.35000 | 49 | 926 |
| 18 | 20.4575 | 4.33780 | 4 | 0.18500 | 20 | 282 |
| 19 | 21.2582 | 4.17619 | 39 | 0.25150 | 206 | 3052 |
| 20 | 22.3268 | 3.97866 | 4 | 0.19640 | 21 | 231 |
| 21 | 23.6590 | 3.75756 | 32 | 0.27800 | 170 | 2696 |
| 22 | 24.4250 | 3.64143 | 4 | 0.23000 | 21 | 245 |
| 23 | 25.0681 | 3-54945 | 36 | 0.37870 | 195 | 3838 |
| 24 | 25.8430 | 3.44475 | 26 | 0.33400 | 138 | 2544 |
| 25 | 26.7610 | 3.32863 | 19 | 0.29400 | 100 | 1581 |
| 26 | 27.4400 | 3.24778 | 15 | 0.39200 | 78 | 1399 |
| 27 | 27.7200 | 3.21561 | 10 | 0.27340 | 51 | 801 |
| 28 | 29.3905 | 3.03653 | 6 | 0.40760 | 34 | 820 |
| 29 | 30.2236 | 2.95470 | 4 | 0.23930 | 19 | 314 |
| 30 | 31.6105 | 2.82815 | 7 | 0.25100 | 37 | 751 |
| 31 | 33.0166 | 2.71085 | 4 | 0.23330 | 19 | 341 |
| 32 | 34.5275 | 2.59561 | 4 | 0.20500 | 19 | 305 |
| 33 | 35.1650 | 2.55000 | 4 | 0.29000 | 19 | 390 |
| 34 | 37.8140 | 2.37723 | 5 | 0.30800 | 25 | 519 |
| 35 | 38.9500 | 2.31047 | 4 | 0.24000 | 22 | 329 |
| 36 | 39.9626 | 2.25423 | 3 | 0.34130 | 18 | 499 |
| 37 | 44.4620 | 2.03599 | 6 | 0.40400 | 32 | 711 |

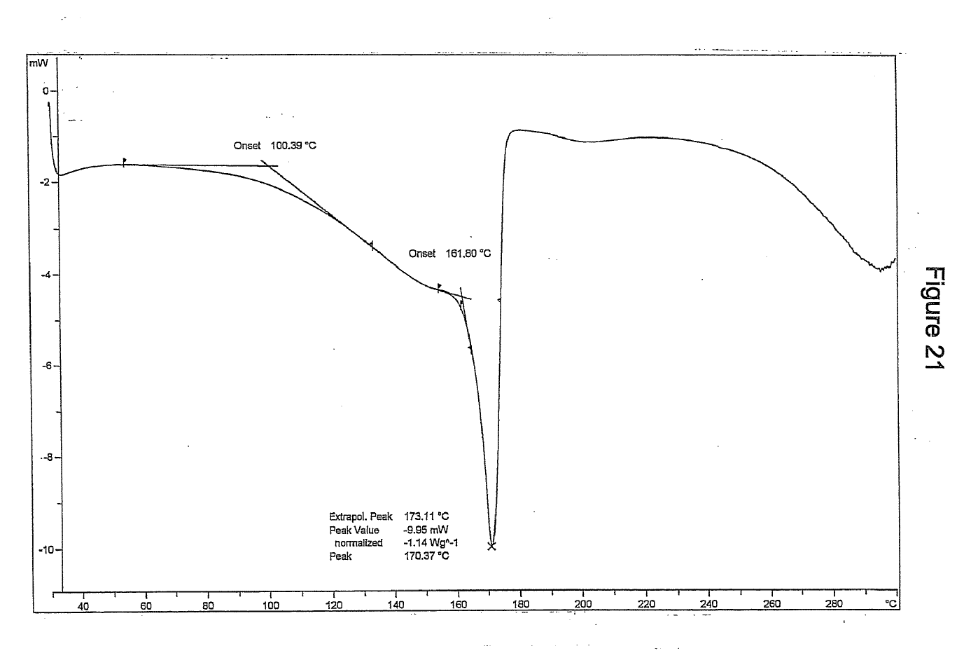

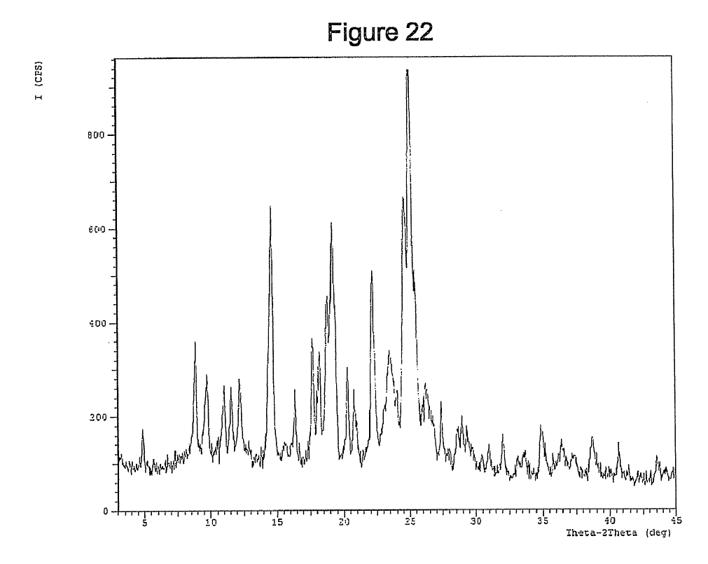

In some embodiments, the present invention provides Form B of Compound 1, having an X-ray diffraction pattern substantially similar to that depicted in FIG. 20. In one aspect, the present invention provides Form B having a DSC pattern substantially similar to that depicted in FIG. 21.

In certain embodiments, the present invention provides Form C of Compound 1. In certain embodiments, the present invention provides Form C of Compound 1, substantially free of other forms of Compound 1. In certain embodiments, Form C is characterized in that it has one or more, two or more, or three or more, peaks in its XRPD pattern selected from those at about 8.8, 9.7, 14.6, 17.7, 18.2, 18.8, 19.2, 22.2, 23.5, 24.6, 25.1 and 25.5 degrees 2-theta.

In other embodiments, Form C of Compound 1 is characterized in that is has substantially all of the peaks in its XRPD pattern listed in Table 3, below.

TABLE 3
XRPD Peaks Form C
# Peak Data List

| peak | 2Theta | d | | FWHM | Intensity | Integrated I [symbol] |
|---|---|---|---|---|---|---|
| no. | (deg) | (A) | I/I1 | (deg) | (Counts) | (Counts) |
| 1 | 4.8810 | 18.08985 | 7 | 0.25800 | 50 | 393 |
| 2 | 8.1200 | 10.87978 | 3 | 1.44000 | 20 | 951 |
| 3 | 8.8412 | 9.99384 | 25 | 0.30050 | 169 | 1169 |
| 4 | 9.6784 | 9.13115 | 20 | 0.37840 | 133 | 1393 |
| 5 | 10.4400 | 3.46668 | 4 | 0.28000 | 26 | 264 |
| 6 | 11.0018 | 8.03556 | 16 | 0.31640 | 106 | 886 |
| 7 | 11.5495 | 7.65570 | 14 | 0.31690 | 97 | 832 |
| 8 | 12.2101 | 7.24295 | 17 | 0.32520 | 115 | 1219 |
| 9 | 14.5762 | 6.07212 | 57 | 0.38720 | 384 | 3973 |
| 10 | 15.5914 | 5.67895 | 3 | 0.30290 | 21 | 160 |
| 11 | 16.3240 | 5.42570 | 11 | 0.26230 | 77 | 563 |
| 12 | 17.6914 | 5.00930 | 25 | 0.27880 | 171 | 1267 |
| 13 | 18.1677 | 4.87903 | 23 | 0.32450 | 158 | 1340 |
| 14 | 18.7600 | 4.72630 | 37 | 0.30440 | 253 | 1650 |
| 15 | 19.1600 | 4.62852 | 53 | 0.56180 | 356 | 4480 |
| 16 | 20.2834 | 4.37464 | 19 | 0.26220 | 127 | 915 |
| 17 | 20.8130 | 4.26450 | 13 | 0.30820 | 91 | 739 |
| 18 | 22.1831 | 4.00411 | 44 | 0.35000 | 295 | 2822 |
| 19 | 23.0400 | 3.85709 | 12 | 0.42000 | 83 | 888 |
| 20 | 23.4800 | 3.78580 | 26 | 0.69540 | 177 | 2913 |
| 21 | 24.0000 | 3.70494 | 15 | 0.00000 | 103 | 0 |
| 22 | 24.6400 | 3.61014 | 67 | 0.40840 | 452 | 5085 |
| 23 | 25.0800 | 3.54779 | 100 | 0.42820 | 676 | 6179 |
| 24 | 25.4800 | 3.49299 | 41 | 0.30760 | 276 | 3218 |
| 25 | 26.2800 | 3.38845 | 19 | 0.00000 | 130 | 0 |
| 26 | 26.7200 | 3.33364 | 11 | 0.37340 | 71 | 1435 |
| 27 | 27.3869 | 3.25396 | 12 | 0.29760 | 83 | 687 |
| 28 | 27.9200 | 3.19303 | 4 | 0.24000 | 29 | 220 |
| 29 | 28.6000 | 3.11864 | 9 | 0.32720 | 63 | 481 |
| 30 | 28.9200 | 3.08485 | 11 | 0.38220 | 74 | 502 |
| 31 | 29.3600 | 3.03961 | 9 | 0.72000 | 59 | 620 |
| 32 | 29.7600 | 2.99966 | 5 | 0.26660 | 32 | 221 |
| 33 | 30.4467 | 2.93355 | 3 | 0.24000 | 21 | 143 |
| 34 | 30.9667 | 2.88547 | 5 | 0.28000 | 37 | 299 |
| 35 | 32.0193 | 2.79297 | 8 | 0.26630 | 51 | 384 |
| 36 | 33.1540 | 2.69993 | 3 | 0.26800 | 23 | 162 |
| 37 | 33.6125 | 2.66414 | 5 | 0.26500 | 35 | 272 |
| 38 | 34.9422 | 2.56574 | 11 | 0.37330 | 77 | 594 |
| 39 | 35.2400 | 2.54474 | 3 | 0.24000 | 23 | 185 |
| 40 | 36.4100 | 2.46561 | 8 | 0.42000 | 51 | 714 |
| 41 | 37.2980 | 2.40892 | 5 | 0.47600 | 33 | 474 |
| 42 | 38.6838 | 2.32575 | 9 | 0.37060 | 63 | 770 |
| 43 | 40.6787 | 2.21618 | 6 | 0.27160 | 42 | 440 |
| 44 | 43.5666 | 2.07574 | 5 | 0.33330 | 34 | 346 |

[symbol]

indicates data missing or illegible when filed

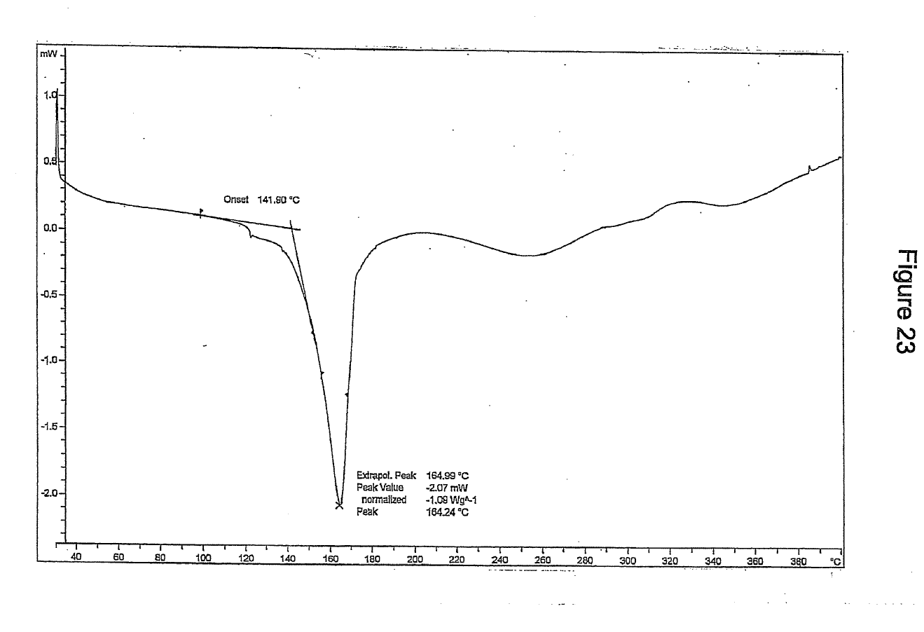

In some embodiments, the present invention provides Form C of Compound 1, having an X-ray diffraction pattern substantially similar to that depicted in FIG. 22. In one aspect, the present invention provides Form C having a DSC pattern substantially similar to that depicted in FIG. 23. In some embodiments, Form C is characterized in that it has a melting point of 164° C.

In certain embodiments, Compound 1 exists in at least one solvate form. In certain embodiments, the present invention provides Form D of Compound 1, as a dimethylacetamide (DMA) solvate. In certain embodiments, the present invention provides Form D of Compound 1.

In certain embodiments, the present invention provides Form D of Compound 1. In certain embodiments, the present invention provides Form D of Compound 1, substantially free of other forms of Compound 1. In certain embodiments, Form D is characterized in that it has one or more, two or more, or three or more, peaks in its XRPD pattern selected from those at about 8.0, 9.8, 13.5, 13.9, 15.9, 16.2, 18.5, 20.7, 21.1, 24.4, 24.6, 25.0 and 26.3 degrees 2-theta.

In other embodiments, Form D of Compound 1 is characterized in that is has substantially all of the peaks in its XRPD pattern listed in Table 4, below.

TABLE 4
XRPD Peaks Form D
# Peak Data List

| peak | 2Theta | d | | FWHM | Intensity | Integrated I [symbol] |
|---|---|---|---|---|---|---|
| no. | (deq) | (A) | I/I1 | (deq) | (Counts) | (Counts) |
| 1 | 3.4057 | 25.92209 | 3 | 0.07730 | 29 | 134 |
| 2 | 3.8639 | 22.84909 | 5 | 0.10220 | 43 | 170 |
| 3 | 4.1679 | 21.18317 | 4 | 0.11590 | 38 | 181 |
| 4 | 4.7200 | 18.70653 | 7 | 0.14320 | 64 | 323 |
| 5 | 4.8878 | 18.06470 | 16 | 0.23690 | 151 | 892 |
| 6 | 5.8724 | 15.03789 | 3 | 0.12710 | 30 | 123 |
| 7 | 6.2103 | 14.22042 | 3 | 0.08290 | 28 | 81 |
| 8 | 7.4800 | 11.80917 | 3 | 0.17460 | 31 | 325 |
| 9 | 7.9647 | 11.09157 | 27 | 0.18420 | 257 | 1530 |
| 10 | 9.5600 | 9.24397 | 6 | 0.11780 | 56 | 248 |
| 11 | 9.7824 | 9.03430 | 32 | 0.16300 | 303 | 1351 |
| 12 | 12.1392 | 7.28509 | 7 | 0.15120 | 64 | 329 |
| 13 | 13.4761 | 6.56523 | 24 | 0.20930 | 224 | 1345 |
| 14 | 13.9025 | 6.36481 | 38 | 0.20770 | 359 | 2011 |
| 15 | 14.2000 | 6.23213 | 10 | 0.14400 | 90 | 503 |
| 16 | 14.6943 | 6.02358 | 11 | 0.16650 | 99 | 439 |
| 17 | 14.9200 | 5.93296 | 6 | 0.20000 | 55 | 352 |
| 18 | 15.5600 | 5.69034 | 6 | 0.11560 | 55 | 206 |
| 19 | 15.8800 | 5.57639 | 22 | 0.23040 | 210 | 1233 |
| 20 | 16.2354 | 5.45511 | 48 | 0.32780 | 448 | 3854 |
| 21 | 17.6400 | 5.02378 | 3 | 0.09340 | 31 | 122 |
| 22 | 17.8753 | 4.95818 | 11 | 0.21880 | 101 | 550 |
| 23 | 18.2000 | 4.87044 | 13 | 0.20000 | 125 | 829 |
| 24 | 18.5331 | 4.78365 | 69 | 0.22010 | 648 | 3878 |
| 25 | 19.8984 | 4.45840 | 3 | 0.21030 | 31 | 185 |
| 26 | 20.3547 | 4.35947 | 8 | 0.17600 | 75 | 344 |
| 27 | 20.6800 | 4.29163 | 20 | 0.17460 | 188 | 1325 |
| 28 | 21.1200 | 4.20320 | 28 | 0.15680 | 258 | 1431 |
| 29 | 21.3600 | 4.15651 | 6 | 0.11420 | 54 | 504 |
| 30 | 21.7516 | 4.08256 | 6 | 0.18330 | 54 | 266 |
| 31 | 22.1622 | 4.00784 | 18 | 0.17170 | 166 | 770 |
| 32 | 23.3487 | 3.80679 | 4 | 0.10250 | 36 | 117 |
| 33 | 23.9794 | 3.70807 | 16 | 0.28120 | 150 | 1108 |
| 34 | 24.3600 | 3.65099 | 25 | 0.15060 | 230 | 821 |
| 35 | 24.6000 | 3.61592 | 40 | 0.20160 | 371 | 2330 |
| 36 | 24.9636 | 3.56407 | 100 | 0.15520 | 936 | 4351 |
| 37 | 25.6374 | 3.47190 | 4 | 0.09000 | 40 | 106 |
| 38 | 26.2824 | 3.38814 | 51 | 0.18760 | 476 | 2724 |
| 39 | 26.7791 | 3.32642 | 14 | 0.19240 | 131 | 810 |
| 40 | 27.3415 | 3.25926 | 11 | 0.23960 | 107 | 637 |
| 41 | 27.8267 | 3.20352 | 3 | 0.24000 | 31 | 242 |
| 42 | 28.4657 | 3.13304 | 5 | 0.20700 | 47 | 302 |
| 43 | 29.5208 | 3.02342 | 15 | 0.14720 | 140 | 678 |
| 44 | 31.0692 | 2.87618 | 5 | 0.26150 | 46 | 515 |
| 45 | 32.6307 | 2.74202 | 3 | 0.11000 | 29 | 202 |
| 46 | 33.4725 | 2.67496 | 3 | 0.18500 | 30 | 259 |
| 47 | 34.7906 | 2.57658 | 3 | 0.37070 | 31 | 494 |
| 48 | 36.8708 | 2.43584 | 4 | 0.16960 | 39 | 240 |
| 49 | 39.0209 | 2.30643 | 5 | 0.18360 | 44 | 210 |

[symbol]

indicates data missing or illegible when filed

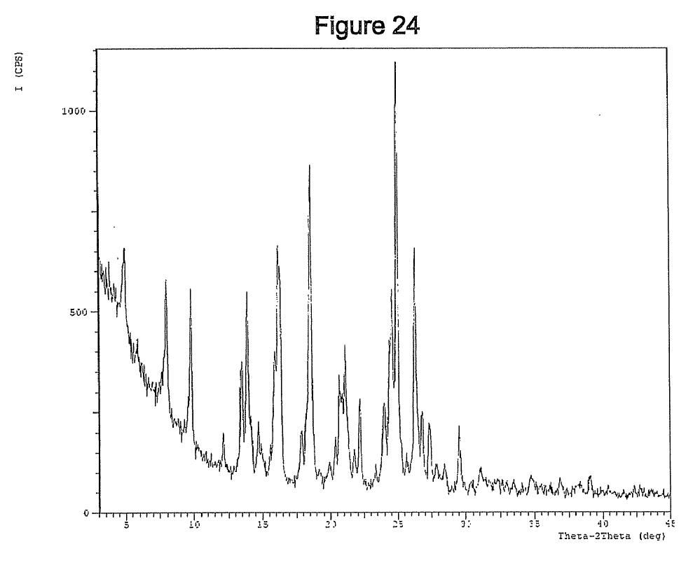

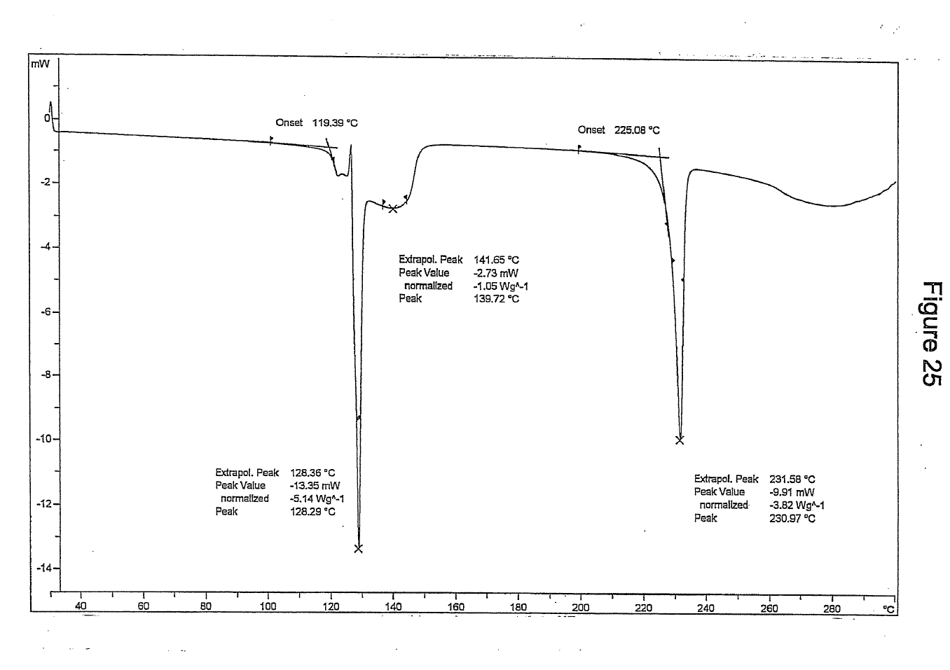

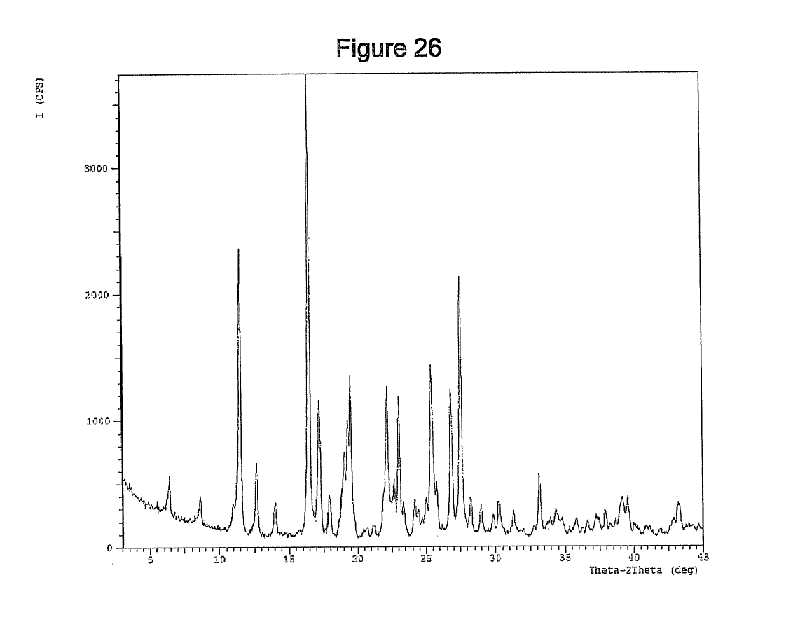

In some embodiments, the present invention provides Form D of Compound 1, having an X-ray diffraction pattern substantially similar to that depicted in FIG. 24. In one aspect, the present invention provides Form D having a DSC pattern substantially similar to that depicted in FIG. 25.

In certain embodiments, Compound 1 exists in at least one solvate form. In certain embodiments, the present invention provides Form E of Compound 1, as a formamide solvate. In certain embodiments, the present invention provides Form E of Compound 1.

In certain embodiments, the present invention provides Form E of Compound 1. In certain embodiments, the present invention provides Form E of Compound 1, substantially free of other forms of Compound 1. In certain embodiments, Form E is characterized in that it has one or more, two or more, or three or more, peaks in its XRPD pattern selected from those at about 11.5, 12.7, 16.5, 17.2, 19.0, 19.3, 19.5, 22.2, 23.0, 25.4, 26.8 and 27.5 degrees 2-theta.

In other embodiments, Form E of Compound 1 is characterized in that is has substantially all of the peaks in its XRPD pattern listed in Table 5, below.

TABLE 5
XRPD Peaks Form E
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 6.4010 | 13.79719 | 7 | 0.19350 | 209 | 1447 |
| 2 | 8.6160 | 10.25454 | 6 | 0.18510 | 182 | 1117 |
| 3 | 11.0000 | 8.03687 | 5 | 0.15680 | 165 | 1206 |
| 4 | 11.5173 | 7.67703 | 61 | 0.17170 | 1833 | 9852 |
| 5 | 12.6787 | 6.97629 | 15 | 0.18410 | 456 | 2594 |
| 6 | 14.0166 | 6.31326 | 7 | 0.18440 | 206 | 1199 |
| 7 | 16.1600 | 5.48039 | 6 | 0.12480 | 174 | 1036 |
| 8 | 16.4704 | 5.37780 | 100 | 0.17070 | 3022 | 14441 |
| 9 | 17.1901 | 5.15423 | 28 | 0.22660 | 838 | 5698 |
| 10 | 17.9375 | 4.94112 | 8 | 0.19160 | 250 | 1377 |
| 11 | 18.8000 | 4.71634 | 8 | 0.18580 | 255 | 3404 |
| 12 | 19.0400 | 4.65742 | 17 | 0.00000 | 526 | 0 |
| 13 | 19.2800 | 4.59999 | 24 | 0.23420 | 725 | 5354 |
| 14 | 19.5200 | 4.54397 | 34 | 0.13880 | 1025 | 5077 |
| 15 | 21.8000 | 4.07360 | 5 | 0.12680 | 143 | 834 |
| 16 | 22.1590 | 4.00841 | 31 | 0.20220 | 951 | 6169 |
| 17 | 22.6800 | 3.91750 | 11 | 0.15560 | 325 | 1876 |
| 18 | 23.0310 | 3.85858 | 31 | 0.16990 | 931 | 4388 |
| 19 | 23.3422 | 3.80784 | 6 | 0.18920 | 196 | 1258 |
| 20 | 24.1450 | 3.68302 | 7 | 0.19960 | 221 | 1156 |
| 21 | 24.4400 | 3.63922 | 4 | 0.12940 | 129 | 645 |
| 22 | 25.0000 | 3.55896 | 7 | 0.18580 | 218 | 1790 |
| 23 | 25.4193 | 3.50120 | 38 | 0.19730 | 1137 | 6420 |
| 24 | 25.8000 | 3.45039 | 9 | 0.11460 | 284 | 1736 |
| 25 | 26.8196 | 3.32149 | 31 | 0.19580 | 943 | 5335 |
| 26 | 27.5266 | 3.23776 | 59 | 0.19200 | 1772 | 9986 |
| 27 | 28.2140 | 3.16042 | 8 | 0.16870 | 232 | 1211 |
| 28 | 28.9581 | 3.08088 | 6 | 0.16830 | 177 | 817 |
| 29 | 29.8444 | 2.99137 | 4 | 0.19820 | 117 | 634 |
| 30 | 30.2385 | 2.95328 | 7 | 0.20110 | 226 | 1411 |
| 31 | 31.2905 | 2.85634 | 5 | 0.17900 | 149 | 1021 |
| 32 | 33.1593 | 2.69951 | 14 | 0.17850 | 420 | 2265 |
| 33 | 33.8499 | 2.64600 | 3 | 0.34390 | 102 | 865 |
| 34 | 34.3901 | 2.60566 | 5 | 0.29280 | 163 | 1100 |
| 35 | 34.7600 | 2.57878 | 3 | 0.20740 | 91 | 613 |
| 36 | 35.7716 | 2.50813 | 3 | 0.22710 | 93 | 808 |
| 37 | 37.3179 | 2.40768 | 3 | 0.34250 | 95 | 1029 |
| 38 | 37.8887 | 2.37271 | 5 | 0.12960 | 156 | 567 |
| 39 | 38.9600 | 2.30990 | 5 | 0.14000 | 144 | 749 |
| 40 | 39.1600 | 2.29856 | 7 | 0.28120 | 216 | 1472 |
| 41 | 39.5226 | 2.27830 | 7 | 0.19870 | 222 | 1253 |
| 42 | 43.2000 | 2.09250 | 7 | 0.20180 | 214 | 1733 |
| 43 | 43.3600 | 2.08515 | 4 | 0.09060 | 131 | 521 |

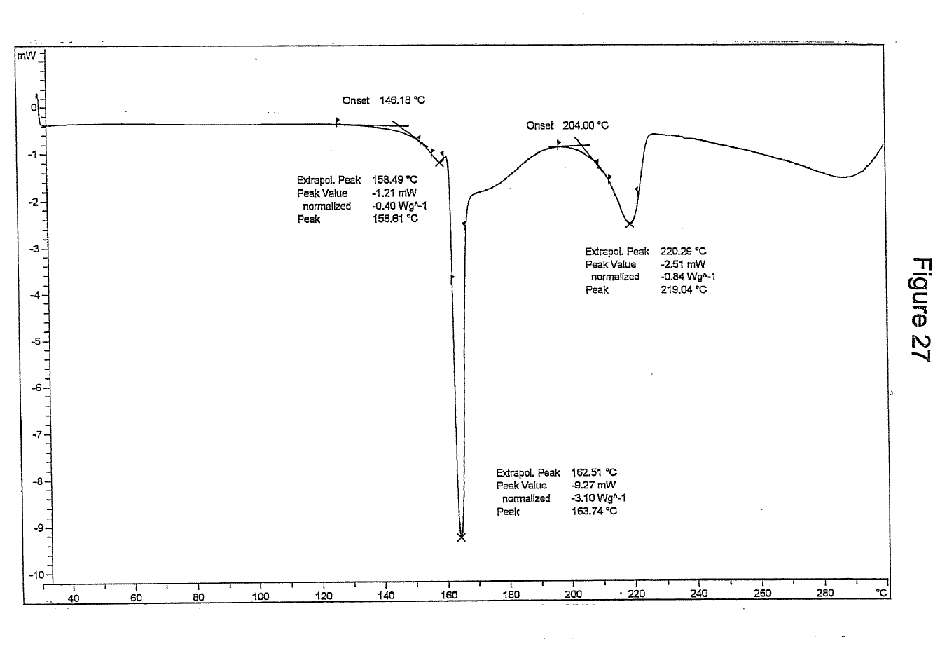

In some embodiments, the present invention provides Form E of Compound 1, having an X-ray diffraction pattern substantially similar to that depicted in FIG. 26. In one aspect, the present invention provides Form E having a DSC pattern substantially similar to that depicted in FIG. 27.

In certain embodiments, the present invention provides Form F of Compound 1. In certain embodiments, the present invention provides Form F of Compound 1, substantially free of other forms of Compound 1. In certain embodiments, Form F is characterized in that it has one or more, two or more, or three or more, peaks in its XRPD pattern selected from those at about 9.8, 11.4, 13.0, 13.3, 17.1, 17.7, 18.0, 19.4 and 19.9 degrees 2-theta.

In other embodiments, Form F of Compound 1 is characterized in that is has substantially all of the peaks in its XRPD pattern listed in Table 6, below.

TABLE 6
XRPD Peaks Form F
# Peak Data List

| peak | 2Theta | d | | FWHM | Intensity | Integrated |
|---|---|---|---|---|---|---|
| no. | (deg) | (A) | I/I1 | (deg) | (Counts) | Int (Counts) |
| 1 | 3.4900 | 25.29614 | 4 | 0.08660 | 12 | 66 |
| 2 | 3.8273 | 23.06751 | 5 | 0.12130 | 15 | 107 |
| 3 | 4.0733 | 21.67492 | 7 | 0.13330 | 21 | 133 |
| 4 | 4.2150 | 20.94657 | 5 | 0.11000 | 16 | 82 |
| 5 | 4.4200 | 19.97551 | 4 | 0.12000 | 11 | 67 |
| 6 | 4.8024 | 18.38575 | 5 | 0.22710 | 16 | 162 |
| 7 | 5.0583 | 17.45617 | 7 | 0.14330 | 21 | 149 |
| 8 | 5.5600 | 15.88211 | 5 | 0.20000 | 14 | 153 |
| 9 | 5.7200 | 15.43821 | 5 | 0.14860 | 15 | 97 |
| 10 | 5.9010 | 14.96507 | 5 | 0.18200 | 14 | 122 |
| 11 | 6.2000 | 14.24403 | 4 | 0.06660 | 11 | 47 |
| 12 | 6.3600 | 13.88604 | 4 | 0.10800 | 12 | 110 |
| 13 | 6.5613 | 13.46046 | 4 | 0.09070 | 12 | 75 |
| 14 | 7.0533 | 12.52260 | 3 | 0.10670 | 10 | 101 |
| 15 | 7.5466 | 11.70510 | 4 | 0.09330 | 11 | 70 |
| 16 | 7.8033 | 11.32061 | 3 | 0.10000 | 10 | 67 |
| 17 | 9.3760 | 9.42496 | 7 | 0.15200 | 21 | 223 |
| 18 | 9.7532 | 9.06129 | 47 | 0.17360 | 136 | 1374 |
| 19 | 10.2750 | 8.60226 | 3 | 0.05000 | 9 | 52 |
| 20 | 10.8591 | 8.14083 | 3 | 0.06830 | 9 | 55 |
| 21 | 11.0400 | 8.00784 | 3 | 0.12000 | 10 | 165 |
| 22 | 11.3803 | 7.76914 | 53 | 0.16070 | 156 | 1467 |
| 23 | 11.8053 | 7.49038 | 5 | 0.12070 | 15 | 153 |
| 24 | 12.5966 | 7.02157 | 4 | 0.11330 | 12 | 84 |
| 25 | 12.8600 | 6.87834 | 6 | 0.13000 | 18 | 125 |
| 26 | 13.0400 | 6.78379 | 22 | 0.17340 | 63 | 723 |
| 27 | 13.3191 | 6.64226 | 100 | 0.19570 | 292 | 2854 |
| 28 | 13.6200 | 6.49619 | 12 | 0.16400 | 35 | 517 |
| 29 | 14.0133 | 6.31473 | 4 | 0.09330 | 11 | 94 |
| 30 | 14.3175 | 6.18124 | 3 | 0.08500 | 9 | 67 |
| 31 | 15.4545 | 5.72895 | 8 | 0.17900 | 24 | 239 |
| 32 | 16.0130 | 5.53037 | 6 | 0.11400 | 17 | 125 |
| 33 | 16.8600 | 5.25440 | 4 | 0.10660 | 13 | 91 |
| 34 | 17.1076 | 5.17890 | 32 | 0.20470 | 92 | 851 |
| 35 | 17.3200 | 5.11587 | 11 | 0.16000 | 33 | 333 |
| 36 | 17.7176 | 5.00195 | 34 | 0.24110 | 98 | 1196 |
| 37 | 18.0408 | 4.91306 | 61 | 0.18070 | 179 | 1706 |
| 38 | 18.6600 | 4.75140 | 11 | 0.17720 | 32 | 310 |
| 39 | 18.9400 | 4.68179 | 14 | 0.24660 | 40 | 536 |
| 40 | 19.4044 | 4.57077 | 24 | 0.37460 | 69 | 1228 |
| 41 | 19.8825 | 4.46193 | 18 | 0.24500 | 54 | 644 |
| 42 | 20.4558 | 4.33815 | 10 | 0.18170 | 29 | 285 |
| 43 | 20.8200 | 4.26308 | 11 | 0.20000 | 33 | 354 |
| 44 | 21.6225 | 4.10664 | 4 | 0.16500 | 11 | 168 |
| 45 | 22.4716 | 3.95335 | 14 | 0.17670 | 42 | 455 |
| 46 | 22.8600 | 3.88706 | 35 | 0.22660 | 101 | 1328 |
| 47 | 23.3512 | 3.80639 | 17 | 0.14250 | 51 | 463 |
| 48 | 23.6786 | 3.75449 | 17 | 0.17070 | 50 | 491 |
| 49 | 24.0066 | 3.70393 | 3 | 0.10670 | 9 | 71 |
| 50 | 24.5450 | 3.62389 | 4 | 0.15000 | 12 | 121 |
| 51 | 24.9606 | 3.56449 | 81 | 0.19450 | 236 | 2340 |
| 52 | 25.2783 | 3.52041 | 15 | 0.15670 | 43 | 380 |
| 53 | 25.6375 | 3.47189 | 24 | 0.24500 | 70 | 841 |
| 54 | 26.2159 | 3.39659 | 43 | 0.18050 | 127 | 1203 |
| 55 | 26.6073 | 3.34750 | 7 | 0.19870 | 20 | 271 |
| 56 | 27.3333 | 3.26022 | 7 | 0.13330 | 19 | 160 |
| 57 | 27.7223 | 3.21535 | 42 | 0.18660 | 122 | 1406 |
| 58 | 28.1550 | 3.16691 | 4 | 0.09000 | 11 | 95 |
| 59 | 28.7100 | 3.10694 | 10 | 0.22000 | 29 | 309 |
| 60 | 29.0600 | 3.07031 | 4 | 0.12000 | 11 | 76 |
| 61 | 31.4680 | 2.84064 | 4 | 0.09600 | 13 | 87 |
| 62 | 33.3325 | 2.68588 | 3 | 0.10500 | 10 | 91 |
| 63 | 34.9433 | 2.56567 | 4 | 0.11330 | 13 | 144 |
| 64 | 36.2200 | 2.47811 | 4 | 0.10000 | 11 | 77 |
| 65 | 36.5580 | 2.45597 | 4 | 0.11600 | 12 | 141 |
| 66 | 41.4066 | 2.17889 | 3 | 0.13330 | 10 | 111 |

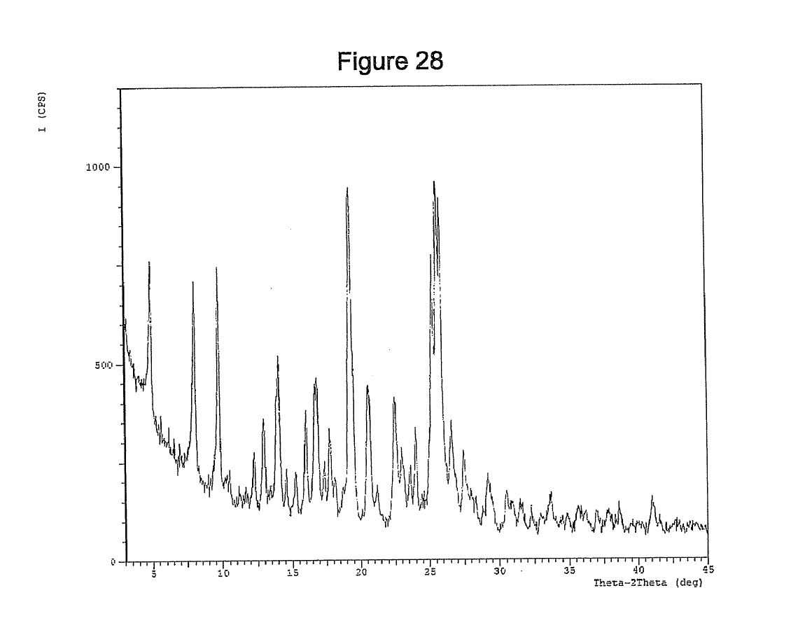

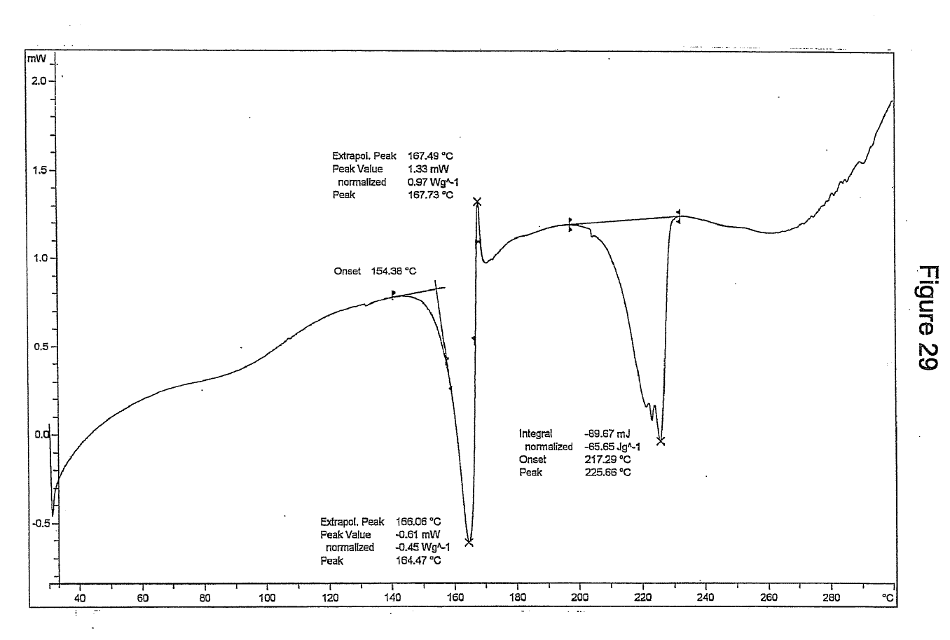

In some embodiments, the present invention provides Form F of Compound 1, having an X-ray diffraction pattern substantially similar to that depicted in FIG. 28. In one aspect, the present invention provides Form F having a DSC pattern substantially similar to that depicted in FIG. 29.

As described above, Compound 1 exists in at least one hydrate form. One such hydrate, i.e., a monohydrate, is referred to herein as Form G. In certain embodiments, the present invention provides Form G of Compound 1. In certain embodiments, the present invention provides Form G of Compound 1, substantially free of other forms of Compound 1. In certain embodiments, Form G is characterized in that it has one or more, two or more, or three or more, peaks in its XRPD pattern selected from those at about 6.2, 11.9, 12.3, 16.7, 18.2, 18.5, 19.2, 22.3, 24.7, 26.0, 26.6 and 27.4 degrees 2-theta.

In other embodiments, Form G of Compound 1 is characterized in that is has substantially all of the peaks in its XRPD pattern listed in Table 7, below.

TABLE 7
XRPD Peaks Form G
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 5.8400 | 15.12125 | 3 | 0.23000 | 41 | 382 |
| 2 | 6.1752 | 14.30117 | 18 | 0.20960 | 234 | 1396 |
| 3 | 8.8573 | 9.97571 | 4 | 0.17630 | 56 | 303 |
| 4 | 11.4000 | 7.75576 | 14 | 0.21940 | 185 | 1662 |
| 5 | 11.8537 | 7.45991 | 58 | 0.23210 | 760 | 5246 |
| 6 | 12.3194 | 7.17893 | 32 | 0.23430 | 422 | 2626 |
| 7 | 13.4638 | 6.57120 | 9 | 0.25760 | 116 | 911 |
| 8 | 14.3628 | 6.16185 | 6 | 0.37910 | 79 | 784 |
| 9 | 16.2800 | 5.44027 | 5 | 0.15200 | 68 | 390 |
| 10 | 16.7011 | 5.30403 | 100 | 0.21830 | 1317 | 8173 |
| 11 | 17.4036 | 5.09148 | 6 | 0.22940 | 80 | 527 |
| 12 | 17.7313 | 4.99812 | 8 | 0.16930 | 100 | 452 |
| 13 | 18.2000 | 4.87044 | 22 | 0.24700 | 291 | 1813 |
| 14 | 18.5425 | 4.78125 | 32 | 0.34900 | 417 | 3488 |
| 15 | 19.2077 | 4.61714 | 36 | 0.40820 | 471 | 5472 |
| 16 | 20.0247 | 4.43056 | 4 | 0.16610 | 54 | 239 |
| 17 | 20.6192 | 4.30414 | 6 | 0.18970 | 77 | 398 |
| 18 | 21.4453 | 4.14017 | 6 | 0.38930 | 78 | 703 |
| 19 | 21.8800 | 4.05889 | 7 | 0.24000 | 94 | 749 |
| 20 | 22.2800 | 3.98692 | 27 | 0.30700 | 356 | 2440 |
| 21 | 22.6000 | 3.93118 | 12 | 0.30720 | 152 | 1755 |
| 22 | 23.8400 | 3.72944 | 9 | 0.22060 | 119 | 1593 |
| 23 | 24.3200 | 3.65691 | 13 | 0.00000 | 172 | 0 |
| 24 | 24.7466 | 3.59483 | 31 | 0.35160 | 411 | 4319 |
| 25 | 25.1600 | 3.53669 | 7 | 0.14940 | 95 | 497 |
| 26 | 25.9972 | 3.42466 | 31 | 0.31320 | 402 | 3349 |
| 27 | 26.6361 | 3.34395 | 16 | 0.21580 | 214 | 1249 |
| 28 | 27.3617 | 3.25690 | 78 | 0.37020 | 1029 | 10166 |
| 29 | 28.1468 | 3.16781 | 9 | 0.25090 | 114 | 1108 |
| 30 | 32.3200 | 2.76767 | 4 | 0.17000 | 48 | 308 |
| 31 | 32.4800 | 2.75440 | 4 | 0.18280 | 55 | 273 |
| 32 | 33.7377 | 2.65454 | 9 | 0.25360 | 120 | 933 |
| 33 | 36.0813 | 2.48731 | 3 | 0.16270 | 41 | 276 |
| 34 | 39.5440 | 2.27712 | 3 | 0.59920 | 41 | 756 |
| 35 | 40.6493 | 2.21772 | 3 | 0.28530 | 40 | 342 |
| 36 | 41.8800 | 2.15535 | 6 | 0.29720 | 75 | 535 |
| 37 | 42.1200 | 2.14362 | 4 | 0.14580 | 49 | 224 |

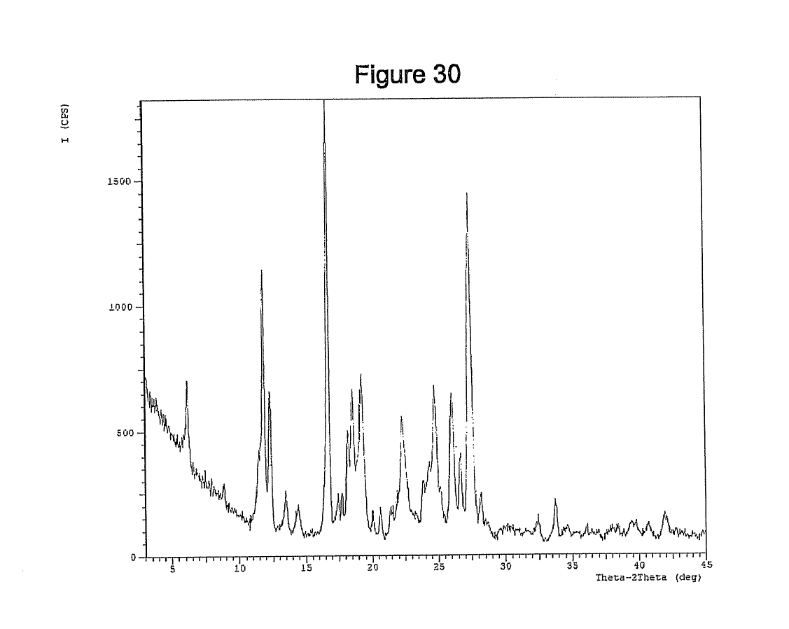

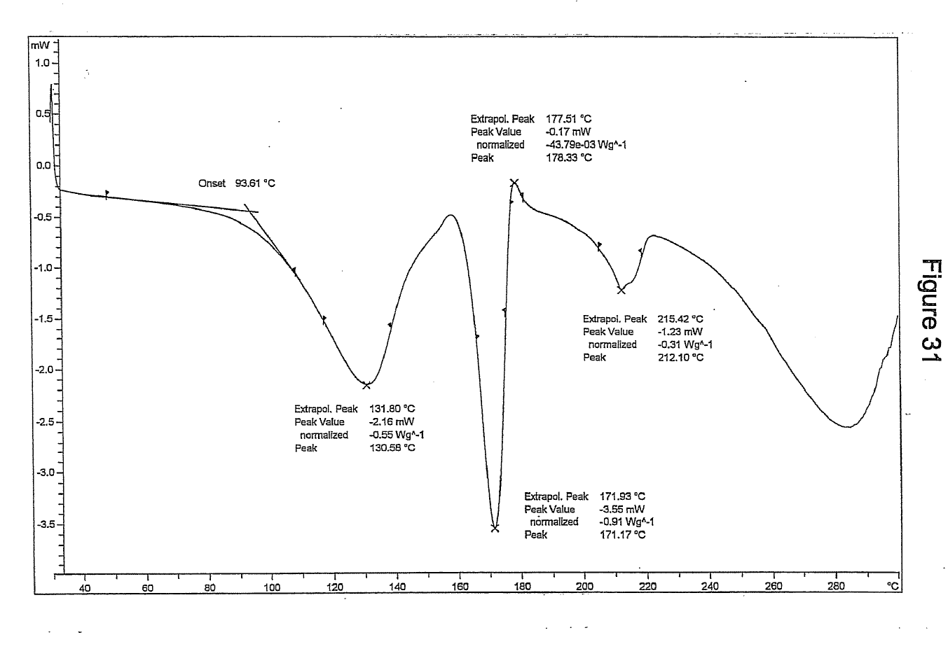

In some embodiments, the present invention provides Form G of Compound 1, having an X-ray diffraction pattern substantially similar to that depicted in FIG. 30. In one aspect, the present invention provides Form G having a DSC pattern substantially similar to that depicted in FIG. 31.

As described above, Compound 1 exists in at least one solvate form. In certain embodiments, the present invention provides Form H of Compound 1, as an ethanol solvate. In certain embodiments, the present invention provides Form H of Compound 1. In certain embodiments, the present invention provides Form H of Compound 1, substantially free of other forms of Compound 1. In certain embodiments, Form H is characterized in that it has one or more, two or more, or three or more, peaks in its XRPD pattern selected from those at about 9.8, 12.2, 13.6, 18.4, 18.7, 19.6, 20.0, 24.5, 24.8 and 28.7 degrees 2-theta.

In other embodiments, Form H of Compound 1 is characterized in that is has substantially all of the peaks in its XRPD pattern listed in Table 8, below.

TABLE 8
XRPD Peaks Form H
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 6.1433 | 14.37536 | 13 | 0.24670 | 217 | 1804 |
| 2 | 9.7574 | 9.05740 | 22 | 0.23840 | 365 | 2877 |
| 3 | 10.6481 | 8.30167 | 12 | 0.23900 | 205 | 1691 |
| 4 | 12.2266 | 7.23321 | 26 | 0.23680 | 436 | 3293 |
| 5 | 13.0800 | 6.76314 | 8 | 0.23320 | 128 | 969 |
| 6 | 13.6072 | 6.50227 | 54 | 0.22920 | 898 | 6297 |
| 7 | 15.4987 | 5.71271 | 4 | 0.18990 | 61 | 349 |
| 8 | 16.2000 | 5.46695 | 6 | 0.17940 | 104 | 522 |
| 9 | 16.4825 | 5.37388 | 14 | 0.25130 | 236 | 1524 |
| 10 | 17.7241 | 5.00013 | 11 | 0.37570 | 182 | 1855 |
| 11 | 18.3698 | 4.82581 | 41 | 0.27630 | 686 | 4682 |
| 12 | 18.7231 | 4.73553 | 70 | 0.23420 | 1161 | 7439 |
| 13 | 19.5600 | 4.53476 | 17 | 0.26900 | 276 | 2156 |
| 14 | 19.9574 | 4.44535 | 21 | 0.30940 | 348 | 2701 |
| 15 | 20.7471 | 4.27790 | 6 | 0.19170 | 104 | 534 |
| 16 | 21.5200 | 4.12597 | 5 | 0.24500 | 81 | 719 |
| 17 | 22.1072 | 4.01769 | 12 | 0.43360 | 205 | 2366 |
| 18 | 22.9431 | 3.87316 | 6 | 0.18740 | 101 | 514 |
| 19 | 23.6000 | 3.76682 | 11 | 0.23280 | 176 | 1882 |
| 20 | 24.1200 | 3.68678 | 9 | 0.00000 | 153 | 0 |
| 21 | 24.5200 | 3.62753 | 37 | 0.25260 | 607 | 4723 |
| 22 | 24.8312 | 3.58277 | 100 | 0.24890 | 1658 | 11193 |
| 23 | 26.2201 | 3.39605 | 37 | 0.27200 | 614 | 4390 |
| 24 | 26.5200 | 3.35833 | 5 | 0.12500 | 76 | 591 |
| 25 | 27.2224 | 3.27325 | 4 | 0.18210 | 68 | 356 |
| 26 | 28.6625 | 3.11198 | 24 | 0.24470 | 403 | 2873 |
| 27 | 29.2400 | 3.05182 | 4 | 0.21340 | 64 | 765 |
| 28 | 30.2624 | 2.95100 | 8 | 0.24120 | 126 | 1073 |
| 29 | 31.8122 | 2.81068 | 3 | 0.16080 | 54 | 429 |
| 30 | 32.4000 | 2.76102 | 8 | 0.21600 | 125 | 558 |
| 31 | 32.6000 | 2.74454 | 6 | 0.42660 | 92 | 1068 |
| 32 | 35.1110 | 2.55380 | 6 | 0.23140 | 94 | 694 |
| 33 | 38.7082 | 2.32434 | 9 | 0.27190 | 150 | 1257 |
| 34 | 41.6466 | 2.16689 | 3 | 0.41330 | 50 | 1003 |

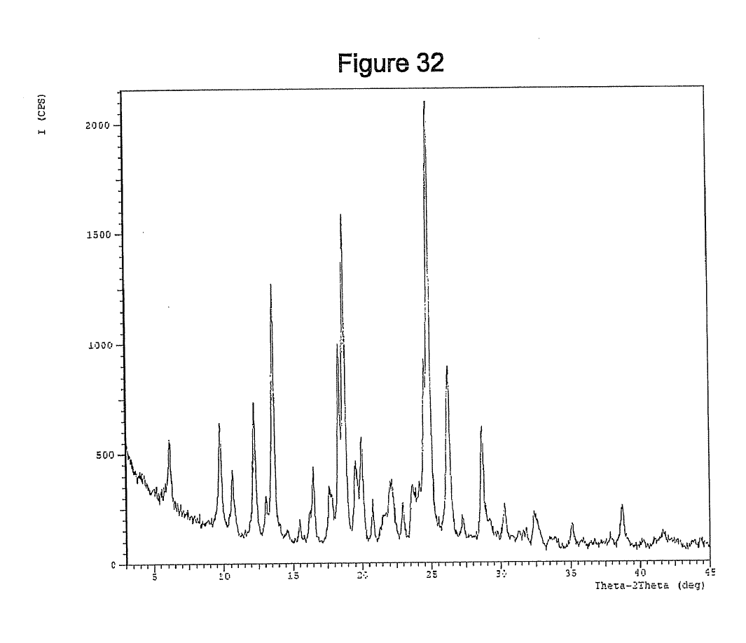

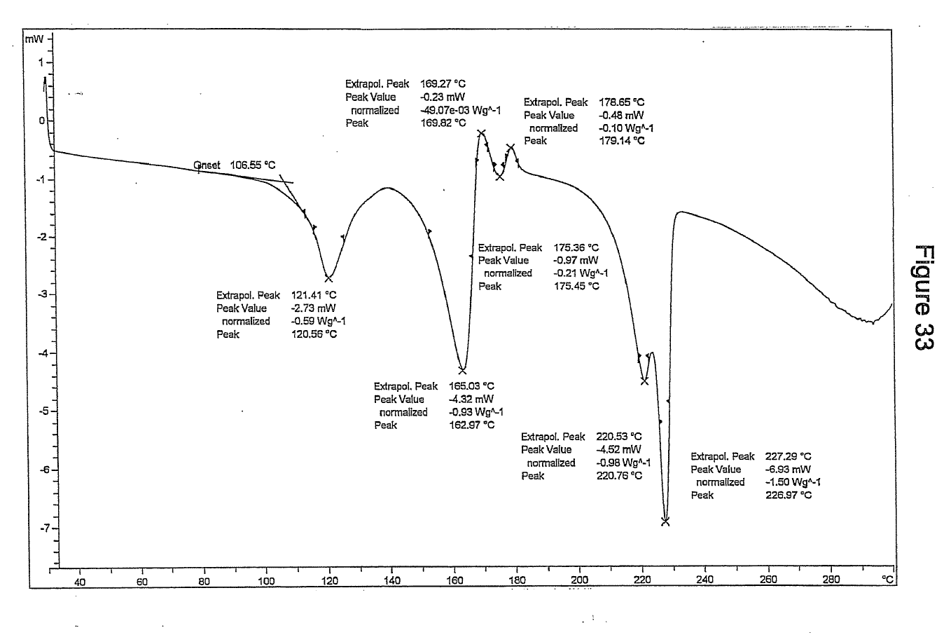

In some embodiments, the present invention provides Form H of Compound 1, having an X-ray diffraction pattern substantially similar to that depicted in FIG. 32. In one aspect, the present invention provides Form H having a DSC pattern substantially similar to that depicted in FIG. 33.

In certain embodiments, the present invention provides Form I of Compound 1, as an acetic acid solvate. In certain embodiments, the present invention provides Form I of Compound 1. In certain embodiments, the present invention provides Form I of Compound 1, substantially free of other forms of Compound 1. In certain embodiments, Form I is characterized in that it has one or more, two or more, or three or more, peaks in its XRPD pattern selected from those at about 9.4, 13.3, 13.7, 17.0, 17.7, 18.8, 19.3, 20.7, 22.1, 22.5, 24.6, 24.8, 25.3, 26.7 and 29.8 degrees 2-theta.

In other embodiments, Form I of Compound 1 is characterized in that is has substantially all of the peaks in its XRPD pattern listed in Table 9, below.

TABLE 9
XRPD Peaks Form I
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 8.9200 | 9.90573 | 4 | 0.25720 | 60 | 800 |
| 2 | 9.4257 | 9.37538 | 24 | 0.20800 | 358 | 2067 |
| 3 | 10.3369 | 8.55089 | 11 | 0.22520 | 163 | 1084 |
| 4 | 10.7191 | 8.24684 | 6 | 0.23820 | 83 | 519 |
| 5 | 13.3200 | 6.64181 | 26 | 0.23920 | 387 | 2662 |
| 6 | 13.6515 | 6.48127 | 79 | 0.22170 | 1176 | 6875 |
| 7 | 14.5370 | 6.08840 | 3 | 0.21400 | 50 | 349 |
| 8 | 15.3723 | 5.75940 | 5 | 0.23040 | 67 | 484 |
| 9 | 17.0254 | 5.20372 | 29 | 0.20730 | 437 | 2597 |
| 10 | 17.7285 | 4.99890 | 26 | 0.20560 | 392 | 2365 |
| 11 | 18.5200 | 4.78700 | 4 | 0.12720 | 64 | 328 |
| 12 | 18.7887 | 4.71915 | 33 | 0.21530 | 486 | 2885 |
| 13 | 19.3060 | 4.59385 | 74 | 0.20590 | 1098 | 6023 |
| 14 | 19.9381 | 4.44961 | 18 | 0.22250 | 264 | 1635 |
| 15 | 20.6868 | 4.29023 | 38 | 0.21260 | 558 | 3138 |
| 16 | 21.3045 | 4.16722 | 4 | 0.19760 | 54 | 300 |
| 17 | 22.1356 | 4.01260 | 30 | 0.22890 | 444 | 2598 |
| 18 | 22.4844 | 3.95113 | 26 | 0.22510 | 393 | 2116 |
| 19 | 22.8862 | 3.88266 | 14 | 0.32180 | 215 | 1729 |
| 20 | 24.1228 | 3.68636 | 8 | 0.19340 | 122 | 751 |
| 21 | 24.6000 | 3.61592 | 25 | 0.33720 | 378 | 2535 |
| 22 | 24.8000 | 3.58721 | 26 | 0.21720 | 392 | 2224 |
| 23 | 25.3370 | 3.51238 | 100 | 0.20940 | 1485 | 9032 |
| 24 | 26.0463 | 3.41832 | 18 | 0.29910 | 260 | 2403 |
| 25 | 26.3600 | 3.37835 | 9 | 0.00000 | 140 | 0 |
| 26 | 26.6957 | 3.33662 | 44 | 0.21240 | 646 | 4156 |
| 27 | 27.8345 | 3.20264 | 11 | 0.17690 | 156 | 883 |
| 28 | 29.7752 | 2.99817 | 31 | 0.18640 | 456 | 2436 |
| 29 | 30.3575 | 2.94197 | 12 | 0.18550 | 172 | 1111 |
| 30 | 30.9729 | 2.88491 | 13 | 0.19280 | 197 | 1181 |
| 31 | 31.9623 | 2.79782 | 14 | 0.20380 | 215 | 1259 |
| 32 | 32.2780 | 2.77118 | 4 | 0.22800 | 52 | 365 |
| 33 | 33.0012 | 2.71208 | 10 | 0.19240 | 144 | 895 |
| 34 | 34.3481 | 2.60875 | 6 | 0.21630 | 90 | 570 |
| 35 | 34.9259 | 2.56690 | 4 | 0.27540 | 64 | 809 |
| 36 | 38.3297 | 2.34642 | 7 | 0.17950 | 108 | 603 |
| 37 | 38.9983 | 2.30772 | 4 | 0.25170 | 59 | 493 |
| 38 | 42.8875 | 2.10702 | 8 | 0.38220 | 117 | 1362 |
| 39 | 43.5852 | 2.07489 | 6 | 0.33280 | 96 | 1142 |

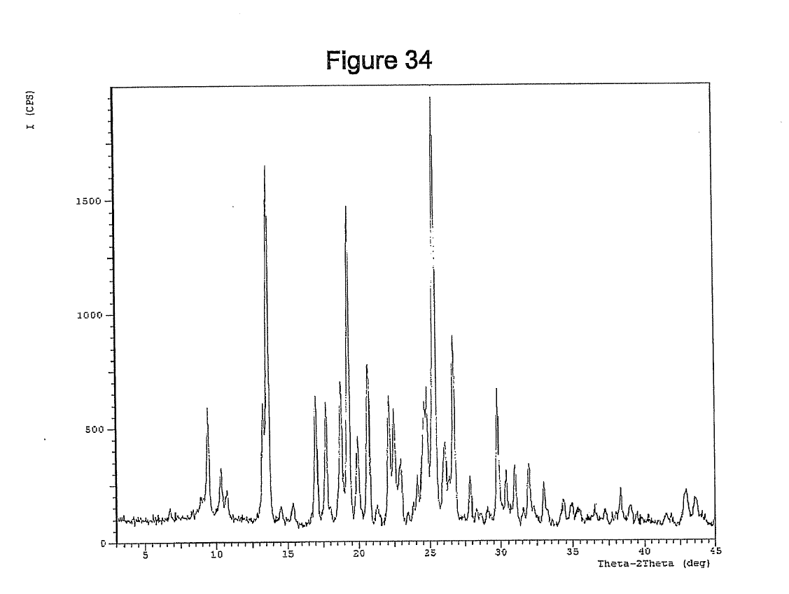

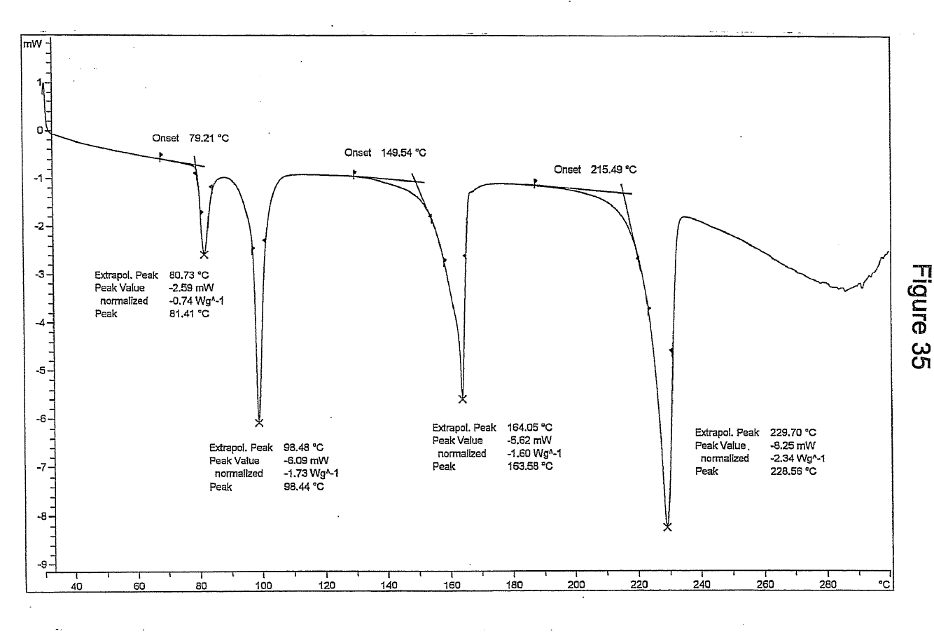

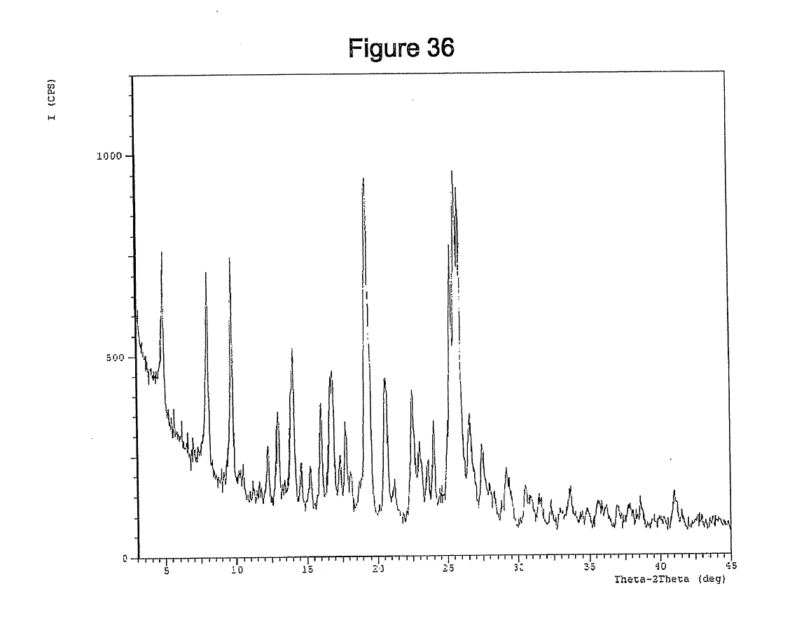

In some embodiments, the present invention provides Form I of Compound 1, having an X-ray diffraction pattern substantially similar to that depicted in FIG. 34. In one aspect, the present invention provides Form I having a DSC pattern substantially similar to that depicted in FIG. 35.

In certain embodiments, the present invention provides Form J of Compound 1, as a dimethylformamide (DMF) solvate. In certain embodiments, the present invention provides Form J of Compound 1. In certain embodiments, the present invention provides Form J of Compound 1, substantially free of other forms of Compound 1. In certain embodiments, Form J is characterized in that it has one or more, two or more, or three or more, peaks in its XRPD pattern selected from those at about 4.9, 8.0, 9.7, 13.0, 14.0, 16.0, 16.8, 17.8, 19.3, 20.6, 22.5, 23.0, 24.0, 25.6, 26.6 and 27.5 degrees 2-theta.

In other embodiments, Form J of Compound 1 is characterized in that is has substantially all of the peaks in its XRPD pattern listed in Table 10, below.

TABLE 10
XRPD Peaks Form J
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 4.8657 | 18.14670 | 29 | 0.34630 | 185 | 1847 |
| 2 | 7.9899 | 11.05664 | 42 | 0.36280 | 271 | 2796 |
| 3 | 9.7224 | 9.08992 | 44 | 0.35060 | 284 | 2575 |
| 4 | 10.3390 | 8.54916 | 4 | 0.37800 | 28 | 246 |
| 5 | 12.2474 | 7.22097 | 9 | 0.29980 | 58 | 464 |
| 6 | 12.9696 | 6.82046 | 19 | 0.33350 | 124 | 1096 |
| 7 | 14.0017 | 6.31994 | 40 | 0.37050 | 259 | 2587 |
| 8 | 14.5855 | 6.06826 | 8 | 0.30100 | 48 | 386 |
| 9 | 15.2405 | 5.80891 | 8 | 0.33240 | 52 | 416 |
| 10 | 16.0174 | 5.52886 | 21 | 0.34150 | 135 | 1157 |
| 11 | 16.7606 | 5.28534 | 41 | 0.39870 | 260 | 2801 |
| 12 | 17.7873 | 4.98251 | 17 | 0.41200 | 108 | 1555 |
| 13 | 19.2987 | 4.59557 | 90 | 0.41810 | 578 | 6531 |
| 14 | 20.5982 | 4.30848 | 36 | 0.35440 | 228 | 2039 |
| 15 | 21.1782 | 4.19178 | 4 | 0.28360 | 26 | 189 |
| 16 | 22.5082 | 3.94701 | 29 | 0.38530 | 188 | 1704 |
| 17 | 23.0000 | 3.86371 | 16 | 0.38580 | 100 | 1161 |
| 18 | 23.9938 | 3.70588 | 18 | 0.32100 | 114 | 1283 |
| 19 | 25.5642 | 3.48168 | 100 | 0.85200 | 640 | 12268 |
| 20 | 26.5885 | 3.34983 | 24 | 0.51240 | 154 | 2130 |
| 21 | 27.4847 | 3.24260 | 17 | 0.38550 | 109 | 946 |
| 22 | 28.0400 | 3.17963 | 6 | 0.65600 | 37 | 680 |
| 23 | 29.2206 | 3.05380 | 12 | 0.45010 | 77 | 899 |
| 24 | 30.6816 | 2.91163 | 7 | 0.63670 | 47 | 630 |
| 25 | 31.5335 | 2.83488 | 5 | 0.28300 | 35 | 260 |
| 26 | 33.6489 | 2.66134 | 8 | 0.38220 | 54 | 682 |
| 27 | 34.8466 | 2.57256 | 4 | 0.30670 | 24 | 258 |
| 28 | 35.7100 | 2.51232 | 7 | 0.50000 | 43 | 731 |
| 29 | 37.0120 | 2.42688 | 5 | 0.29600 | 30 | 226 |
| 30 | 37.8091 | 2.37752 | 5 | 0.42970 | 31 | 355 |
| 31 | 38.6020 | 2.33049 | 5 | 0.36400 | 29 | 280 |
| 32 | 41.0166 | 2.19870 | 8 | 0.35330 | 53 | 590 |

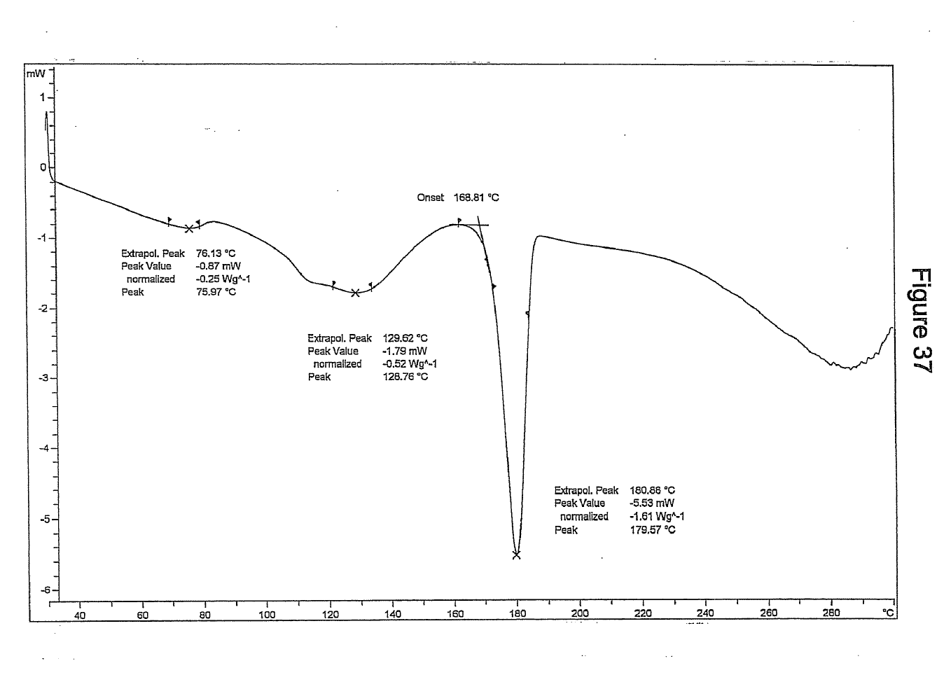

In some embodiments, the present invention provides Form J of Compound 1, having an X-ray diffraction pattern substantially similar to that depicted in FIG. 36. In one aspect, the present invention provides Form J having a DSC pattern substantially similar to that depicted in FIG. 37.

In certain embodiments, Compound 1 exists in at least one solvate form. In certain embodiments, the present invention provides Form K of Compound 1, as an N-methylpyrrolidinone (NMP) solvate. In certain embodiments, the present invention provides Form K of Compound 1. In certain embodiments, the present invention provides Form K of Compound 1, substantially free of other forms of Compound 1. In certain embodiments, Form K is characterized in that it has one or more, two or more, or three or more, peaks in its XRPD pattern selected from those at about 13.4, 13.9, 15.3, 16.8, 18.1, 21.3, 22.8, 24.5, 24.9, 25.2 and 28.6 degrees 2-theta.

In other embodiments, Form K of Compound 1 is characterized in that is has substantially all of the peaks in its XRPD pattern listed in Table 11, below.

TABLE 11
XRPD Peaks Form K
8 Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 7.2989 | 12.10177 | 2 | 0.27780 | 81 | 766 |
| 2 | 9.1200 | 9.68894 | 4 | 0.27660 | 168 | 1221 |
| 3 | 9.4273 | 9.37379 | 5 | 0.23870 | 185 | 1097 |
| 4 | 11.3052 | 7.82058 | 9 | 0.21180 | 351 | 2319 |
| 5 | 12.7054 | 6.96169 | 2 | 0.18910 | 87 | 549 |
| 6 | 13.3906 | 6.60695 | 17 | 0.20920 | 696 | 4039 |
| 7 | 13.8884 | 6.37124 | 12 | 0.25300 | 488 | 2959 |
| 8 | 14.1600 | 6.24964 | 3 | 0.24620 | 132 | 1359 |
| 9 | 14.7313 | 6.00853 | 7 | 0.26770 | 260 | 2172 |
| 10 | 15.2912 | 5.78976 | 14 | 0.20500 | 564 | 3272 |
| 11 | 16.0400 | 5.52112 | 9 | 0.21860 | 372 | 2103 |
| 12 | 16.2400 | 5.45357 | 9 | 0.18220 | 356 | 2043 |
| 13 | 16.8481 | 5.25808 | 10 | 0.20160 | 386 | 2142 |
| 14 | 17.8000 | 4.97898 | 4 | 0.24820 | 156 | 1284 |
| 15 | 18.1023 | 4.89651 | 10 | 0.21230 | 398 | 2165 |
| 16 | 18.6400 | 4.75646 | 2 | 0.11200 | 65 | 217 |
| 17 | 18.9234 | 4.68586 | 8 | 0.21730 | 321 | 1786 |
| 18 | 19.2927 | 4.59699 | 3 | 0.19170 | 136 | 784 |
| 19 | 20.0618 | 4.42246 | 7 | 0.26330 | 272 | 1832 |
| 20 | 20.3600 | 4.35835 | 4 | 0.22840 | 143 | 998 |
| 21 | 20.7196 | 4.28351 | 4 | 0.19650 | 160 | 899 |
| 22 | 21.3167 | 4.16486 | 11 | 0.23700 | 443 | 2856 |
| 23 | 21.8800 | 4.05889 | 5 | 0.32500 | 184 | 1440 |
| 24 | 22.0400 | 4.02979 | 6 | 0.22500 | 228 | 993 |
| 25 | 22.6000 | 3.93118 | 9 | 0.22580 | 358 | 1929 |
| 26 | 22.8400 | 3.89041 | 10 | 0.17520 | 394 | 2202 |
| 27 | 23.3200 | 3.81141 | 2 | 0.10160 | 72 | 322 |
| 28 | 23.6740 | 3.75521 | 9 | 0.23040 | 355 | 2343 |
| 29 | 24.4800 | 3.63337 | 12 | 0.39480 | 481 | 4516 |
| 30 | 24.8800 | 3.57585 | 40 | 0.16060 | 1586 | 8250 |
| 31 | 25.1653 | 3.53596 | 100 | 0.25070 | 3997 | 25825 |
| 32 | 26.4744 | 3.36401 | 4 | 0.28110 | 152 | 1221 |
| 33 | 26.8800 | 3.31416 | 3 | 0.15800 | 118 | 520 |
| 34 | 27.1600 | 3.28062 | 2 | 0.14860 | 63 | 510 |
| 35 | 27.8012 | 3.20640 | 3 | 0.19440 | 135 | 660 |
| 36 | 28.2000 | 3.16196 | 3 | 0.13340 | 107 | 356 |
| 37 | 28.5717 | 3.12166 | 10 | 0.27960 | 389 | 3164 |
| 38 | 29.4758 | 3.02794 | 2 | 0.16300 | 79 | 498 |
| 39 | 30.1628 | 2.96052 | 3 | 0.27810 | 128 | 1093 |
| 40 | 30.7126 | 2.90876 | 3 | 0.18220 | 123 | 687 |
| 41 | 32.2838 | 2.77069 | 2 | 0.35670 | 82 | 1143 |
| 42 | 32.9136 | 2.71910 | 2 | 0.22190 | 67 | 381 |
| 43 | 33.2846 | 2.68963 | 2 | 0.31920 | 65 | 488 |
| 44 | 34.1600 | 2.62268 | 3 | 0.31060 | 103 | 865 |
| 45 | 34.5268 | 2.59566 | 3 | 0.24370 | 116 | 877 |

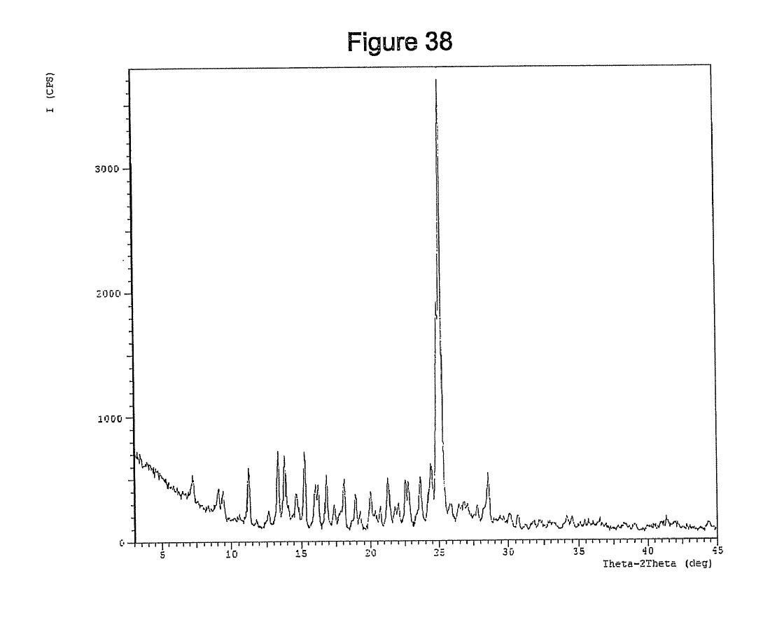

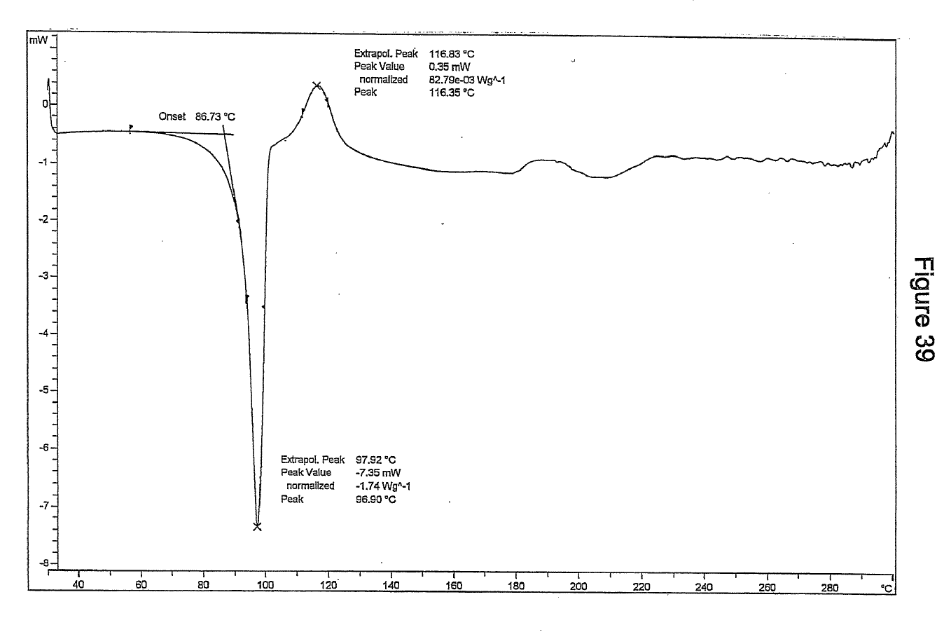

In some embodiments, the present invention provides Form K of Compound 1, having an X-ray diffraction pattern substantially similar to that depicted in FIG. 38. In one aspect, the present invention provides Form K having a DSC pattern substantially similar to that depicted in FIG. 39.

In certain embodiments, the present invention provides Form L of Compound 1, as a DMF solvate. In certain embodiments, the present invention provides Form L of Compound 1. In certain embodiments, the present invention provides Form L of Compound 1, substantially free of other forms of Compound 1. In certain embodiments, Form L is characterized in that it has one or more, two or more, or three or more, peaks in its XRPD pattern selected from those at about 8.6, 13.1, 13.6, 14.3, 15.5, 17.1, 19.7, 21.0, 21.4, 22.0, 23.8, 25.7, 26.0, 26.3, 27.4 and 36.7 degrees 2-theta.

In other embodiments, Form L of Compound 1 is characterized in that is has substantially all of the peaks in its XRPD pattern listed in Table 12, below.

TABLE 12
XRPD Peaks Form L
# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 8.6002 | 10.27334 | 17 | 0.21800 | 718 | 4810 |
| 2 | 11.2161 | 7.88251 | 3 | 0.20180 | 142 | 1221 |
| 3 | 13.0528 | 6.77717 | 7 | 0.21970 | 306 | 2104 |
| 4 | 13.5995 | 6.50593 | 7 | 0.30570 | 292 | 2295 |
| 5 | 14.3277 | 6.17687 | 8 | 0.22090 | 334 | 1928 |
| 6 | 14.6858 | 6.02704 | 5 | 0.23490 | 204 | 1295 |
| 7 | 15.4534 | 5.72935 | 8 | 0.35800 | 339 | 3103 |
| 8 | 17.1047 | 5.17978 | 21 | 0.21840 | 890 | 5746 |
| 9 | 18.3513 | 4.83063 | 3 | 0.25170 | 133 | 1308 |
| 10 | 19.0382 | 4.65786 | 6 | 0.20600 | 248 | 1347 |
| 11 | 19.7005 | 4.50274 | 15 | 0.24070 | 608 | 3802 |
| 12 | 20.1200 | 4.40979 | 4 | 0.25040 | 146 | 1548 |
| 13 | 21.0352 | 4.21995 | 20 | 0.28640 | 835 | 6052 |
| 14 | 21.3600 | 4.15651 | 13 | 0.39160 | 559 | 5224 |
| 15 | 22.0233 | 4.03280 | 35 | 0.21280 | 1450 | 8712 |
| 16 | 22.4000 | 3.96583 | 3 | 0.07640 | 131 | 496 |
| 17 | 23.7667 | 3.74078 | 28 | 0.23710 | 1157 | 8319 |
| 18 | 24.8000 | 3.58721 | 4 | 0.20260 | 180 | 1287 |
| 19 | 25.0800 | 3.54779 | 5 | 0.14180 | 191 | 943 |
| 20 | 25.6800 | 3.46624 | 7 | 0.25760 | 279 | 3153 |
| 21 | 25.9600 | 3.42949 | 7 | 0.00000 | 285 | 0 |
| 22 | 26.3309 | 3.38201 | 100 | 0.20210 | 4157 | 24328 |
| 23 | 27.3729 | 3.25559 | 11 | 0.19670 | 456 | 3726 |
| 24 | 28.8086 | 3.09653 | 4 | 0.24270 | 172 | 1606 |
| 25 | 31.9416 | 2.79959 | 4 | 0.27400 | 166 | 2105 |
| 26 | 34.4525 | 2.60108 | 3 | 0.20350 | 130 | 1099 |
| 27 | 35.3329 | 2.53826 | 3 | 0.22000 | 127 | 933 |
| 28 | 36.0702 | 2.48805 | 4 | 0.24830 | 169 | 1309 |
| 29 | 36.4400 | 2.46365 | 4 | 0.22900 | 148 | 814 |
| 30 | 36.7351 | 2.44453 | 7 | 0.24530 | 272 | 1775 |
| 31 | 38.4402 | 2.33993 | 4 | 0.21420 | 181 | 1064 |
| 32 | 39.1276 | 2.30039 | 3 | 0.23610 | 129 | 1947 |

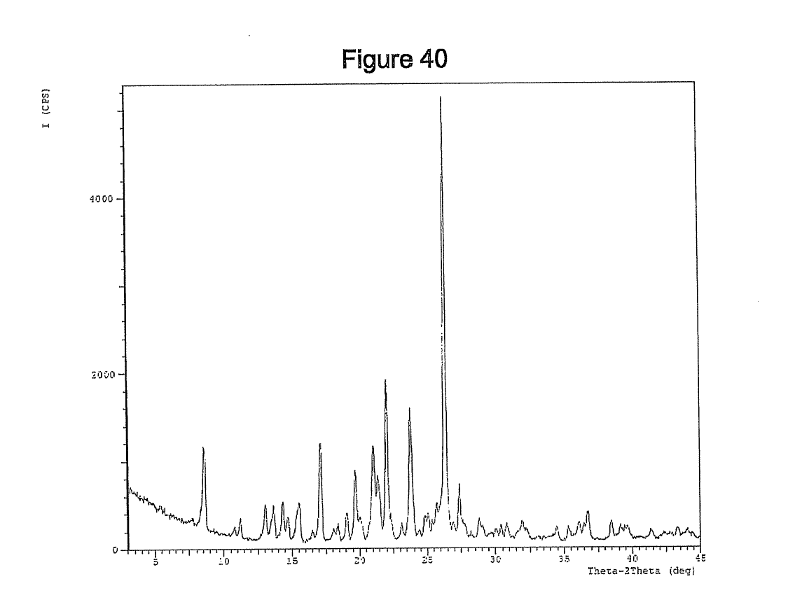

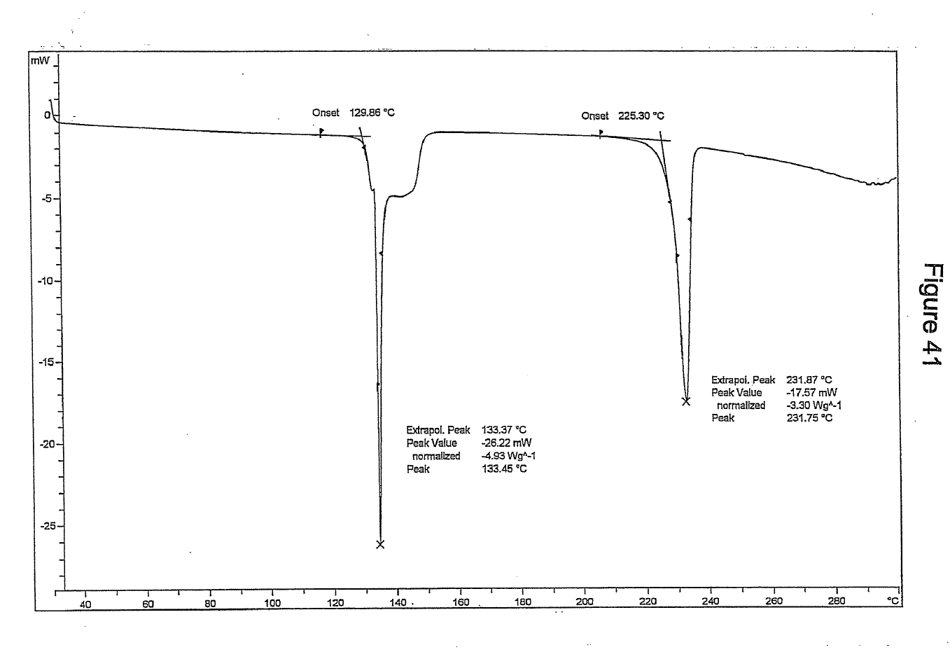

In some embodiments, the present invention provides Form L of Compound 1, having an X-ray diffraction pattern substantially similar to that depicted in FIG. 40. In one aspect, the present invention provides Form L having a DSC pattern substantially similar to that depicted in FIG. 41.

In another embodiment, the present invention provides Compound 1 as an amorphous solid. Amorphous solids are well known to one of ordinary skill in the art and are typically prepared by such methods as lyophilization, melting and precipitation from supercritical fluid, among others.

In certain embodiments, the present invention provides a composition comprising Form A of Compound 1 and at least one or more other solid forms of Compound 1. In some embodiments, the present invention provides a composition comprising Form A and Form B. In other embodiments, the present invention provides a composition comprising Form A and amorphous Compound 1.

### (3) Formulations

The present invention provides formulations and methods of administration of Compound 1. In certain embodiments, the present invention provides formulations that are suitable for parenteral administration of Compound 1. Formulations provided for parenteral administration include sterile solutions for injection, sterile suspensions for injection, sterile emulsions, and dispersions. In some embodiments, Compound 1 is formulated for intravenous administration. In some embodiments, Compound 1 is formulated for intravenous administration at a concentration of about 0.5 to about 5.0 mg/mL.

In certain embodiments, the solubility of Compound 1 in a formulation can be improved by the addition of solubilizing agents. Solubilizing agents are known to one skilled in the art and include cyclodextrins, nonionic surfactants, and the like. Cyclodextrins include, for example, sulfobutyl ether beta-cyclodextrin, sodium salt (e.g., Captisol®). Exemplary nonionic surfactants include Tween®-80 and PEG-400. Other illustrative formulations of Compound 1 of the present invention include 10%/30%/60%, 5%/30%/65%, and 2.5%/30%/67.5%, respectively, of Tween-80, PEG-400, and water.

In certain embodiments, the present invention provides a composition comprising Compound 2 or a pharmaceutically acceptable salt thereof, and a solubilizing agent.

In some embodiments, the present invention provides a composition comprising Compound 2 or a pharmaceutically acceptable salt thereof, and a cyclodextrin.

In some embodiments, the present invention provides a composition comprising Compound 2 or a pharmaceutically acceptable salt thereof, and a sulfobutyl ether beta-cyclodextrin, sodium salt.

In certain embodiments, the present invention provides a composition comprising Compound 1, and a solubilizing agent.

In some embodiments, the present invention provides a composition comprising Compound 1, and a cyclodextrin.

In some embodiments, the present invention provides a composition comprising Compound 1, and a sulfobutyl ether beta-cyclodextrin, sodium salt.

### Additional Components

In some embodiments, formulations may comprise one or more additional agents for modification and/or optimization of release and/or absorption characteristics. For example, incorporation of buffers, co-solvents, diluents, preservatives, and/or surfactants may facilitate dissolution, absorption, stability, and/or improved activity of active compound(s), and may be utilized in formulations of the invention. In some embodiments, where additional agents are included in a formulation, the amount of additional agents in the formulation may optionally include: buffers about 10% to about 90%, co-solvents about 1% to about 50%, diluents about 1% to about 10%, preservative agents about 0.1% to about 8%, and/or surfactants about 1% to about 30%, based upon total weight of the formulation, as applicable.

Suitable co-solvents (i.e., water-miscible solvents) are known in the art. For example, suitable co-solvents include, but are not limited to ethyl alcohol, propylene glycol.

Physiologically acceptable diluents may optionally be added to improve product characteristics. Physiologically acceptable diluents are known in the art and include, but are not limited to, sugars, inorganic salts and amino acids, and solutions of any of the foregoing. Representative examples of acceptable diluents include dextrose, mannitol, lactose, and sucrose, sodium chloride, sodium phosphate, and calcium chloride, arginine, tyrosine, and leucine, and the like, and aqueous solutions thereof.

Suitable preservatives are known in the art, and include, for example, benzyl alcohol, methyl paraben, propyl paraben, sodium salts of methyl paraben, thimerosal, chlorobutanol, and phenol. Suitable preservatives include but are not limited to: chlorobutanol (0.3-0.9% W/V), parabens (0.01-5.0% W/V), thimerosal (0.004-0.2% W/V), benzyl alcohol (0.5-5% W/V), phenol (0.1-1.0% W/V), and the like.

Suitable surfactants are also known in the art and include, e.g., poloxamer, polyoxyethylene ethers, polyoxyethylene sorbitan fatty acid esters polyoxyethylene fatty acid esters, polyethylene glycol fatty acid esters, polyoxyethylene hydrogenated castor oil, polyoxyethylene alkyl ether, polysorbates, cetyl alcohol, glycerol fatty acid esters (e.g., triacetin, glycerol monostearate, and the like), polyoxymethylene stearate, sodium lauryl sulfate, sorbitan fatty acid esters, sucrose fatty acid esters, benzalkonium chloride, polyethoxylated castor oil, and docusate sodium, and the like, and combinations thereof. In some embodiments the formulation may further comprise a surfactant.

In certain embodiments, the present invention provides dosage forms including unit dose forms, dose-concentrates, etc. for parenteral administration wherein the dosage forms comprise Compound 1. Parenteral administration of provided formulations may include any of intravenous injection, intravenous infusion, intradermal, intralesional, intramuscular, subcutaneous injection, or depot administration of a unit dose. A unit dose may or may not constitute a single “dose” of active compound(s), as a prescribing doctor may choose to administer more than one, less than one, or precisely one unit dose in each dose (i.e., each instance of administration). For example, unit doses may be administered once, less than once, or more than once a day, for example, once per week, twice per week, once every other day (QOD), once per day, or 2, 3 or 4 times per day, or 1 or 2 times per day.

### (4) Pharmaceutical Uses and Administration

As described above, Compound 1 is an inhibitor of Aurora kinases. As such, it is useful for treating diseases or conditions mediated by one or more Aurora kinases. Such diseases include, for example, cancers. In other embodiments of the methods provided herein, the cancer being treated is selected from the group consisting of bladder cancer, brain cancer, breast cancer, cervical cancer, colon cancer, esophageal cancer, head and neck cancer, leukemia, liver cancer, lung cancer (e.g., small cell and non-small cell lung cancers), lymphoma, melanoma, myeloma, neuroendocrine cancer (e.g., neuroblastoma), ovarian cancer, pancreatic cancer, prostate cancer, renal cancer, sarcoma, skin cancer, stomach cancer, testicular cancer, thyroid cancer, and uterine cancer.

In certain embodiments, the patient has a solid tumor. For example, the method may be used to treat cancers of the brain, colon, lung, prostate, ovary, breast, cervix, and skin. In one embodiment, the lung cancer is a non-small cell lung cancer (NSCLC). In another embodiment, the skin cancer is a melanoma.

In other embodiments, the patient has a hematological tumor. In another embodiment, the patient has a lymphoma or leukemia. In certain embodiments the patient's hematological tumor is a mantle cell lymphoma (MCL), Non-Hodgkin's lymphoma (NHL), Hodgkin's disease, acute myelogenous leukemia (AML), chronic myelogenous leukemia (CML), chronic lymphocytic leukemia (CLL), or acute lymphoblastic lymphoma (ALL).

The invention is also directed to methods of treating cancer, comprising administering specific doses of Compound 1. These doses may be administered once or more than once. In one embodiment, the dose or doses are administered according to schedules described herein. Compositions of compounds formulated to contain the appropriate amount of compound so that the dose is readily administered are also envisaged.

In one aspect, the invention is directed to a method of treating cancer comprising administering to a patient Compound 1 or a composition thereof (e.g., a provided formulation herein) with a frequency of at least once every three weeks. In one embodiment, Compound 1 or a composition thereof is administered once every three weeks. In another embodiment, Compound 1 or a composition thereof is administered once every two weeks. In another embodiment, Compound 1 or a composition thereof is administered once per week. In another embodiment, Compound 1 or a composition thereof is administered twice per week. In another embodiment, the compound is administered daily.

In another embodiment, Compound 1 is administered to the patient in at least one cycle of once a day for five days. In another embodiment Compound 1 is administered in two cycles of once a day for five days, with at least one day between the two cycles wherein the compound is not administered. In another embodiment, Compound 1 is administered in at least two cycles, with two, three, four, five, six, seven, or eight days off between the two cycles. In another embodiment, Compound 1 is administered in at least two cycles, with nine days off between the two cycles.

The invention is also directed to methods of treating cancer comprising administering specific doses of Compound 1. Such doses may be administered once or more than once. In one embodiment, such dose or doses are administered according to schedules described herein. Compositions of compounds formulated to contain the appropriate amount of compound so that the dose is readily administered are also envisaged.

In another aspect, the invention is directed to a method for treating cancer in a patient, comprising administering to a patient having a cancer an effective amount of Compound 1.

In another aspect, the invention is directed to a method for treating cancer in a patient comprising administering to a patient having cancer a dose of about 10 mg/m²-3000 mg/m² of Compound 1. The dose may be administered as a composition comprising the dose of Compound 1 and one or more pharmaceutically acceptable carriers, diluents, or excipients.

In one embodiment, the dose is administered once a week. In another embodiment the dose administered once a week is 240 mg/m²-2000 mg/m². In another embodiment, the dose administered once a week is about 480 mg/m²-1800 mg/m². In another embodiment, the dose administered once a week is about 480 mg/m²-1500 mg/m². In another embodiment, the dose administered once a week is about 480 mg/m²-1200 mg/m². In another embodiment, the dose administered once a week is about 750 mg/m²-1500 mg/m². In another embodiment, the dose administered once a week is about 960 mg/m²-1200 mg/m².

In another embodiment, the dose is administered once a week for three weeks.

In another embodiment, the method of treating cancer comprises administering to a patient a dose of 30 mg/m²-2000 mg/m² of Compound 1 administered in a cycle of once a week for three weeks, wherein there is at least one day off between cycles. In another embodiment, the method of treating cancer comprises administering to a patient a dose of 30 mg/m²-750 mg/m² of Compound 1 administered in a cycle of once a week for three weeks, wherein there is at least one day off between cycles. In another aspect, the invention is directed to a method of treating cancer comprising administering to a patient a dose of 60 mg/m²-750 mg/m² of Compound 1 administered in a cycle of once a week for three weeks, wherein there is at least one day off between cycles. In one embodiment, Compound 1 is administered on Day 1, Day 8, and Day 15 of three week cycle, with 7 days off between cycles. In other words, Compound 1 is administered on Day 1, Day 8, and Day 15 of a 21 day cycle, with 7 days off between cycles. In another embodiment, the dose administered on Day 1, Day 8, and Day 15 of the three week cycle with 7 days off between cycles is 200 mg/m²-600 mg/m². In another embodiment, the dose administered on Day 1, Day 8, and Day 15 of the three week cycle with 7 days off between cycles is 300 mg/m²-500 mg/m². In another embodiment, the dose administered on Day 1, Day 8, and Day 15 of the three week cycle with 7 days off between cycles is 350 mg/m²-450 mg/m². In another embodiment the dose administered on Day 1, Day 8, and Day 15 of the three week cycle with 7 days off between cycles is 300 mg/m²-400 mg/m². In another embodiment, the dose administered on Day 1, Day 8, and Day 15 of the three week cycle with 7 days off between cycles is 400 mg/m²-500 mg/m². In another embodiment, the dose administered on Day 1, Day 8, and Day 15 of the three week cycle with 7 days off between cycles is 500 mg/m²-600 mg/m².

In another aspect, the invention is directed to a method comprising administering to a patient a dose of 30 mg/m²-300 mg/m² of Compound 1. In one embodiment, the dose is administered once per day. In another embodiment, the dose administered once per day is 100 mg/m²-300 mg/m². In another embodiment the dose administered once per day is 150 mg/m²-250 mg/m². In another embodiment, the dose administered once per day is 100 mg/m²-200 mg/m². In another embodiment, the dose administered once per day is 200 mg/m²-300 mg/m². In other embodiments the doses are administered once per day for five days.

### (5) Combination Therapy

It will also be appreciated that Compound 1 and pharmaceutically acceptable compositions comprising Compound 1 can be employed in complementary combination therapies with other active agents or medical procedures. Thus, Compound 1 and pharmaceutically acceptable compositions thereof can be administered concurrently with, prior to, or subsequent to, one or more other desired active agents or medical procedures. The particular combination of therapies (agents or procedures) to employ in a combination regimen will take into account compatibility of the desired therapeutics and/or procedures and the desired therapeutic effect to be achieved. It will also be appreciated that the therapies employed may achieve a desired effect for the same disorder (for example, Compound 1 may be administered concurrently with another active agent used to treat the same disorder), or they may achieve different effects (e.g., control of any adverse effects). Non-limiting examples of such agents and procedures include surgery, radiotherapy (e.g., gamma-radiation, neutron beam radiotherapy, electron beam radiotherapy, proton therapy, brachytherapy, and systemic radioisotopes), endocrine therapy, biologic response modifiers (interferons, interleukins, and tumor necrosis factor (TNF) to name a few examples), hyperthermia and cryotherapy, agents to attenuate any adverse effects (e.g., antiemetic agents), and other approved chemotherapeutic anticancer agents.

Examples of chemotherapeutic anticancer agents that may be used as second active agents in combination with Compound 1 include, but are not limited to, alkylating agents (e.g., mechlorethamine, chlorambucil, cyclophosphamide, melphalan, ifosfamide), antimetabolites (e.g., methotrexate), other aurora kinase inhibitors, purine antagonists and pyrimidine antagonists (e.g., 6-mercaptopurine, 5-fluorouracil, cytarabine, gemcitabine), spindle poisons (e.g., vinblastine, vincristine, vinorelbine, paclitaxel), podophyllotoxins (e.g., etoposide, irinotecan, topotecan), antibiotics (e.g., doxorubicin, daunorubicin, bleomycin, mitomycin), nitrosoureas (e.g., carmustine, lomustine), inorganic ions (e.g., platinum complexes such as cisplatin, carboplatin), enzymes (e.g., asparaginase), hormones (e.g., tamoxifen, leuprolide, flutamide, and megestrol), topoisomerase H inhibitors or poisons, EGFR (Her1, ErbB-1) inhibitors (e.g., gefitinib), antibodies (e.g., rituximab), IMIDs (e.g., thalidomide, lenalidomide), various targeted agents (e.g., HDAC inhibitors such as vorinostat), Bcl-2 inhibitors, VEGF inhibitors); proteasome inhibitors (e.g., bortezomib), cyclin dependent kinase (cdk) inhibitors (e.g. seliciclib), and dexamethasone.

Some specific anticancer agents that can be used in combination with Compound 1 include, but are not limited to: azacitidine (e.g., Vidaza®); bortezomib (e.g., Velcade®); capecitabine (e.g., Xeloda®); carboplatin (e.g., Paraplatin®); cisplatin (e.g., Platinol®); cyclophosphamide (e.g., Cytoxan®, Neosar®); cytarabine (e.g., Cytosar®), cytarabine liposomal (e.g., DepoCyt®), cytarabine ocfosfate or other formulations of the active moiety; doxorubicin, doxorubicin hydrochloride (e.g., Adriamycin®), liposomal doxorubicin hydrochloride (e.g., Doxil®); fludarabine, fludarabine phosphate (Fludara®); 5-fluorouracil (e.g., Adrucil®); gefitinib (e.g., Iressa®); gemcitabine hydrochloride (e.g., Gemzar®); irinotecan (CPT-11, camptothecin-11), irinotecan hydrochloride (e.g., Camptosar®); lenalidomide (e.g., Revlimid®); melphalan (e.g., Alkeran®); paclitaxel (e.g., Taxol®); paclitaxel protein-bound (e.g., Abraxane®); rituximab (e.g., Rituxan®); vorinostat (e.g., Zolinza®).

Other anticancer agents that can be used in combination with Compound 1 include, but are not limited to: acivicin; aclarubicin; acodazole hydrochloride; acronine; adalimumab (e.g., Humire); adozelesin; alitretinoin (e.g., Panretin®); altretamine (hexamethylmelamine; e.g., Hexylen®); ambomycin; ametantrone acetate; aminoglutethimide (e.g., Cytadren®); amonafide malate (e.g., Xanafide®); amsacrine; anastrozole (e.g., Arimidee); anthramycin; asparaginase (e.g., Kidrolase®, Elspar®); asperlin; azetepa; azotomycin; batimastat; benzodepa; bevacizumab (e.g., Avastin®); bexarotene (e.g., Targetin®); bicalutamide (e.g., Casodex®); bisantrene hydrochloride; bisnafide dimesylate; bizelesin; bleomycin sulfate (e.g., Blenoxane®); brequinar sodium; bropirimine; busulfan (e.g., Busulfex®, Myleran®); CD20 antibodies such as ofatumumab; CD23 antibodies such as lumiliximab; CD52 antibodies such as alemtuzumab (e.g., Campath®); CD80 antibodies such as galiximab; cactinomycin; calusterone; caracemide; carbetimer; carmustine (e.g., BiCNU®); carmustine implant (e.g., Gliadel® wafer); carubicin hydrochloride; carzelesin; cedefingol; celecoxib (COX-2 inhibitor, e.g., Celebrex®); chlorambucil (e.g., Leukeran®); cirolemycin; cladribine (e.g., Leustatin®); clofarabine; cloretazine; crisnatol; crisnatol mesylate; 4-hydroperoxycyclophosphamide; dacarbazine (e.g., DTIC-Dome®); dactinomycin (e.g., Cosmegen®); dasatanib (e.g., Sprycel®); daunorubicin hydrochloride (e.g., Cerubidine), liposomal daunorubicin citrate (e.g., DaunoXome®); decitabine (e.g., Dacogen®); denileukin diftitox (e.g., Ontak®); dexormaplatin; dezaguanine, dezaguanine mesylate; diaziquone; droloxifene, droloxifene citrate; dromostanolone propionate; duazomycin; edatrexate; edrecolomab (Panorex®); eflornithine, eflornithine hydrochloride; elsamitrucin; enloplatin; enpromate; epipropidine; epirubicin hydrochloride (e.g., Ellence®); erbulozole; erlotinib (e.g., Tarceva®); esorubicin hydrochloride; estramustine, estramustine phosphate sodium (e.g., Emcyt®), estramustine analogues; etanidazole; etoposide (VP-16; e.g., Toposar®), etoposide phosphate (e.g., Etopophos®); etoprine; fadrozole hydrochloride; fazarabine; fenretinide; floxuridine (e.g., FUDR®); fluorocitabine; flutamide (e.g., Eulexin®); fosquidone; fostriecin, fostriecin sodium; G250 monoclonal antibody; galiximab; gefitinib (e.g., Iressa®); gemtuzumab ozogamicin (Mylotarg®); goserelin acetate (Zoladex®); hydroxyurea (e.g., Droxia®, Hydrea®); ibritumomab tiuxetan (e.g., Zevalin®)+¹¹¹In or ⁹⁰Yt; idarubicin, idarubicin hydrochloride (e.g., Idamycin®); ifosfamide (e.g., Ifex®); ilmofosine; iproplatin; lanreotide, lanreotide acetate; lapatinib (e.g., Tykerb®); letrozole (e.g., Femara®); leuprolide acetate (e.g., Eligard®, Viadur®); liarozole, liarozole hydrochloride; CD33 antibodies such as lintuzumab; lometrexol, lometrexol sodium; lomustine (e.g., CeeNe); losoxantrone, losoxantrone hydrochloride; masoprocol; maytansine; mechlorethamine (nitrogen mustard, mustine), mechlorethamine hydrochloride (e.g., Mustargen®); megestrol acetate (e.g., Megace®); melengestrol acetate; menogaril; mercaptopurine (e.g., Purinethol®); methotrexate sodium (e.g., Rheumatrex®); metoprine; meturedepa; mitindomide; mitocarcin; mitocromin; mitogillin; mitomalcin; mitomycin (Mutamycin), mitomycin analogues; mitosper; mitotane; mitoxantrone, mitoxantrone hydrochloride (e.g., Novantrone®); mycophenolic acid; nelarabine (Arranon®); nocodazole; nogalamycin; ormaplatin; oxisuran; panitumumab (e.g., Vectibix®); pegaspargase (PEG-L-asparaginase; e.g., Oncaspar®); peliomycin; pemetrexed (e.g., Alimta®); pentamustine; peplomycin sulfate; perfosfamide; pertuzumab (e.g. Omnitarg®); pipobroman; piposulfan; piroxantrone hydrochloride; plicamycin; plomestane; porfimer sodium; porfiromycin; prednimustine; procarbazine hydrochloride (e.g., Matulane®); puromycin; puromycin hydrochloride; pyrazofurin; R-roscovitine (seliciclib); riboprine; safingol; safingol hydrochloride; semustine; simtrazene; sorafenib (e.g., Nexavar®); sparfosate sodium; sparsomycin; spirogermanium hydrochloride; spiromustine; spiroplatin; streptonigrin; streptozocin (e.g., Zanosar®); sulofenur; sunitinib malate (e.g., Sutent®); talisomycin; tecogalan sodium; tegafur; teloxantrone hydrochloride; temoporfin; temozolomide (e.g., Temodar®); teniposide (e.g., Vumon; teroxirone; testolactone; thalidomide (e.g., Thalomid®); thiamiprine; thioguanidine; 6-thioguanine; thiotepa (e.g., Thioplex®); tiazofurin; tipifamib (e.g., Zarnestra®); tirapazamine; topotecan (e.g., Hycamtin®); toremifene, toremifene citrate (e.g., Fareston®); tositumomab+¹³¹I (e.g., Bexxar®); trastuzumab (e.g., Herceptin®); trestolone acetate; triciribine, triciribine phosphate; trimetrexate, trimetrexate glucuronate; triptorelin; troxacitabine (e.g., Troxatyl®); tubulozole hydrochloride; uracil mustard; uredepa; vapreotide; verteporfin; vinblastine sulfate (e.g., Velban®); vincristine (leurocristine) sulfate (e.g., Vincasar®); vindesine, vindesine sulfate; vinepidine sulfate; vinglycinate sulfate; vinleurosine sulfate; vinorelbine tartrate (e.g., Navelbine®); vinrosidine sulfate; vinzolidine sulfate; vorozole; zeniplatin; zinostatin, zinostatin stimalamer; and zorubicin (rubidazone) hydrochloride.

Other anticancer agents that can be used in combination with Compound 1 include, but are not limited to: 20-epi-1,25-dihydroxyvitamin D3; 5-ethynyluracil; abiraterone acetate; acylfulvene, (hydroxymethyl)acylfulvene; adecypenol; ALL-TK antagonists; ambamustine; amidox; amifostine; aminolevulinic acid; amrubicin; anagrelide (e.g., Agrylin®); andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; arsenic trioxide (e.g., Trisenox®); asulacrine; atamestane; atrimustine; axinastatin 1; axinastatin 2; axinastatin 3; azasetron; azatoxin; azatyrosine; baccatin III derivatives; balanol; BCR/ABL antagonists; benzochlorins; benzoylstaurosporine; beta lactam derivatives; beta-alethine; betaclamycin B; betulinic acid; bFGF inhibitor; bisantrene; bisaziridinylspermine; bisnafide; bistratene A; brefeldin A or its prodrug breflate; budotitane; buthionine sulfoximine; calcipotriol; calphostin C; camptothecin derivatives (e.g., irinotecan); carboxamide-amino-triazole; carboxyamidotriazole; CaRest M3; CARN 700; cartilage-derived inhibitor; casein kinase inhibitors; castanospermine; cecropin B; cetrorelix; chlorins; chloroquinoxaline sulfonamide; cicaprost; cis-porphyrin; clarithromycin (e.g., Biaxin®); clomifene analogues; clotrimazole; collismycin A; collismycin B; combretastatin A4, combretastatin analogues; conagenin; crambescidin 816; cryptophycin 8; cryptophycin A derivatives; curacin A; cyclopentanthraquinones; cycloplatam; cypemycin; cytolytic factor; cytostatin; dacliximab (daclizumab; e.g., Zenapax®); dehydrodidemnin B; deslorelin; dexamethasone (e.g., Decadron®); dexifosfamide; dexrazoxane; dexverapamil; didemnin B; didox; diethylnorspermine; dihydro-5-azacytidine; 9-dihydrotaxol; dioxamycin; diphenyl; docetaxel (e.g., Taxotere®); docosanol; doxifluridine; duocarmycin SA; ebselen; ecomustine; edelfosine; edrecolomab; elemene; emitefur; epristeride; estrogen agonists; estrogen antagonists; exemestane (e.g., Aromasin®); fadrozole; filgrastim; finasteride; flavopiridol (alvocidib); flezelastine; fluasterone; fluorodaunorunicin hydrochloride; forfenimex; formestane; fotemustine; gadolinium texaphyrin; gallium nitrate; galocitabine; ganciclovir; ganirelix; gelatinase inhibitors; glutathione inhibitors; hepsulfam; heregulin; hexamethylene bisacetamide; hypericin; ibandronic acid; idoxifene; idramantone; ilomastat; imatinib mesylate (e.g., Gleevec®); imiquimod (e.g., Aldara®), and other cytokine inducers; immunostimulant peptides; insulin-like growth factor-1 receptor inhibitor; interferon agonists; interferons such as interferon alpha (e.g., Intron® A); pegylated interferon alfa-2b (e.g., PegIntron®); interleukins such as IL-2 (aldesleukin, e.g., Proleukin®); iobenguane; iododoxorubicin; 4-ipomeanol; iroplact; irsogladine; isobengazole; isohomohalicondrin B; jasplakinolide; kahalalide F; lamellarin-N triacetate; leinamycin; lenograstim; lentinan sulfate; leptolstatin; leukemia inhibiting factor; leukocyte alpha interferon; leuprolide+estrogen+progesterone; leuprorelin; levamisole; linear polyamine analogue; lipophilic disaccharide peptide; lipophilic platinum compounds; lissoclinamide 7; lobaplatin; lombricine; lonidamine; loxoribine; lurtotecan; lutetium texaphyrin; lysofylline; lytic peptides; maitansine; mannostatin A; marimastat; maspin; matrilysin inhibitors; matrix metalloproteinase inhibitors; merbarone; meterelin; methioninase; metoclopramide; MIF inhibitor; mifepristone (e.g., Mifeprex®); miltefosine; mirimostim; mitoguazone; mitolactol; mitonafide; mitotoxin fibroblast growth factor-saporin; mofarotene; cetuximab (e.g., Erbitux®); human chorionic gonadotrophin; monophosphoryl lipid A+mycobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide (e.g., Nilandron®); nisamycin; nitric oxide modulators; nitroxide antioxidants (e.g., tempol); nitrullyn; oblimersen (Genasense®); 06-benzylguanine; octreotide (e.g., Sandostatin®); octreotide acetate (e.g., Sandostatin LAR®); okicenone; oligonucleotides; onapristone; oracin; osaterone; oxaliplatin (e.g., Eloxatin®); oxaunomycin; paclitaxel analogues; paclitaxel derivatives; palauamine; palmitoylrhizoxin; panaxytriol; panomifene; parabactin; pazelliptine; peldesine; pentosan polysulfate sodium; pentostatin (e.g., Nipent®); pentrozole; perflubron; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum-triamine complex; propyl bis-acridone; prostaglandin J2; proteasome inhibitors; protein A-based immune modulator; protein kinase C inhibitors, including microalgal PKC inhibitors; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed (e.g., Tomudex®); ras farnesyl protein transferase inhibitors; ras inhibitors; ras-GAP inhibitor; retelliptine demethylated; rhenium (Rel86); rhizoxin; ribozymes; RII retinamide; rohitukine; romurtide; roquinimex; rubiginone B1; ruboxyl; saintopin; SarCNU; sarcophytol A; Sdi 1 mimetics; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; splenopentin; spongistatin 1; squalamine; steroids (e.g., prednisone, prednisolone); stipiamide; stromelysin inhibitors; sulfinosine; sulindac; superactive vasoactive intestinal peptide antagonist; suradista; suramin; swainsonine; tallimustine; tamoxifen, tamoxifen citrate (e.g., Nolvadex®), tamoxifen methiodide; tauromustine; tazarotene; tellurapyrylium; telomerase inhibitors; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; titanocene bichloride; topsentin; translation inhibitors; tretinoin (all-trans retinoic acid, e.g., Vesanoid®); triacetyluridine; turosteride; tyrosine kinase inhibitors; tyrphostins; UBC inhibitors; ubenimex; urogenital sinus-derived growth inhibitory factor; urokinase receptor antagonists; variolin B; velaresol; veramine; verdins; vinxaltine; vitaxin; zanoterone; and zilascorb.

For a more comprehensive discussion of updated cancer therapies see, The Merck Manual, Seventeenth Ed. 1999. See also the National Cancer Institute (NCl) website (http://www.cancer.gov/drugdictionary/) for a comprehensive list of oncology medicaments suitable as second active agents, and the U.S. Food and Drug Administration (FDA) website for a list of the FDA-approved oncology medicaments.

In other embodiments, the second active agent is a supportive care agent, such as an antiemetic agent or erythropoiesis stimulating agents. Specific antiemetic agents include, but are not limited to, phenothiazines, butyrophenones, benzodiazapines, corticosteroids, serotonin antagonists, cannabinoids, and NK1 receptor antagonists. Examples of phenothiazine antiemetic agents include, but are not limited to, prochlorperazine and trimethobenzamide. Examples of butyrophenone antiemetic agents include, but are not limited to, haloperidol. Examples of benzodiazapine antiemetic agents include, but are not limited to, lorazepam. Examples of corticosteroid antiemetic agents include, but are not limited to, dexamethasone. Examples of serotonin receptor (5-HT3 receptor) antagonist antiemetic agents include, but are not limited to, dolasetron mesylate (e.g., Anzemet®), granisetron (e.g., Kytril®), itasetron, ondansetron (e.g., Zofran®), palonosetron (e.g., Aloxi®) ramosetron, tropisetron (e.g., Navoban®), batanopride, dazopride, renzapride. Examples of cannabinoid antiemetic agents include, but are not limited to, dronabinol. Examples of NK1 receptor antagonists include, but are not limited to, aprepitant (e.g., Emend®).

Other supportive care agents include agents that stimulate erythropoiesis or other hematopoietic processes, such as epoetin alfa (e.g., Epogen®, Procrit®); G-CSF and recombinant forms such as filgrastim (e.g., Neupogen®), pegfilgrastim (e.g., Neulasta®), and lenofilgrastim; darbepoetin alfa (e.g., Aranesp®); and GM-CSF and recombinant forms such as sargramostim (e.g., Leukine®) or molgramostim. Other supportive care agents include chemoprotectant agents such as amifostine (e.g., Ethyol®), dexrazoxane (e.g., Zinecard®), leucovorin (folinic acid), and mesna (e.g., Mesnex®); thrombopoeitic growth factors such as interleukin-11 (IL-11, oprelvekin, e.g., Neumega®); bisphosphonates such as pamidronate disodium (e.g., Aredia®), etidronate disodium (e.g., Didronel®) and zoledronic acid (e.g., Zometa®); and TNF antagonists, such as infliximab (e.g., Remicade®).

Tumor lysis syndrome (TLS) may be expected in the treatment of hematologic cancers, and supportive care treatment(s) to mitigate or prevent TLS or its component symptoms may be administered to patients treated with Compound 1 according to the invention. Treatments suitable for preventing or mitigating TLS (or any of the symptoms thereof, including hyperkalemia, hyperphosphatemia, hyperuricemia, hypocalcemia, and acute renal failure), include, for example, allopurinol (e.g., Zyloprim®), rasburicase (e.g., Elitek®), and sodium polystyrene sulfonate (e.g., Kayexalate®).

Doses and dosing regimens of Compound 1 together with other active moieties and combinations thereof should depend on the specific indication being treated, age and condition of a patient, and severity of adverse effects, and may be adjusted accordingly by those of skill in the art. Examples of doses and dosing regimens for other active moieties can be found, for example, in Physician's Desk Reference, and will require adaptation for use in the methods of the invention.

While the active moieties mentioned herein as second active agents may be identified as free active moieties or as salt forms (including salts with hydrogen or coordination bonds) or other as non-covalent derivatives (e.g., chelates, complexes, and clathrates) of such active moieties, it is to be understood that the given representative commercial drug products are not limiting, and free active moieties, or salts or other derivative forms of the active moieties may alternatively be employed. Accordingly, reference to an active moiety should be understood to encompass not just the free active moiety but any pharmacologically acceptable salt or other derivative form that is consistent with the specified parameters of use.

### (6) Methods for Preparing Compound 1

In one aspect, the present invention provides methods for preparing a Compound 1, according to the steps depicted in Scheme I.

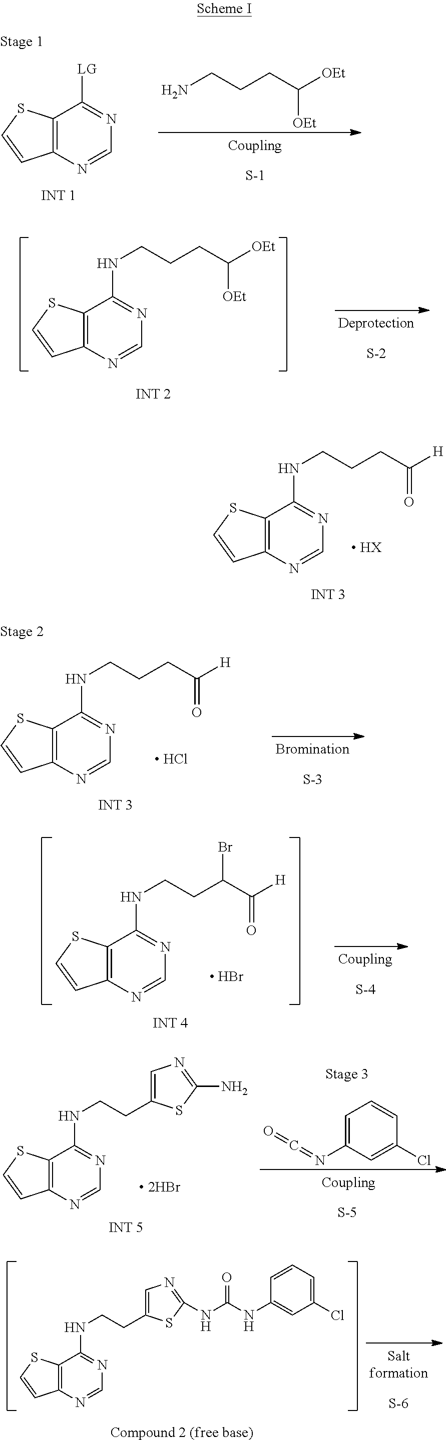

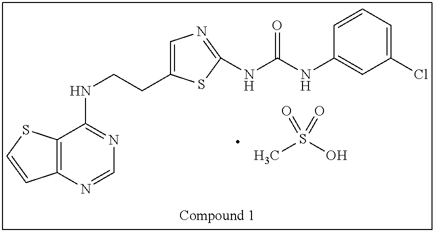

In Scheme I above, LG and HX are as defined below and in classes and subclasses as described herein.

In one aspect, the present invention provides methods for preparing INT5, Compound 2 and Compound 1, according to the steps depicted in Scheme I above. In certain embodiments, the present invention provides a method for preparing Compound 2 comprising the steps of providing INT5 and coupling INT5 with 3-chlorophenyl-isocyanate to form Compound 2.

As depicted in step S-1, a compound of formula INT1 is coupled to aminobutyraldehyde diethyl actetal via a displacement of the LG moiety of formula INT1 to form INT2, where LG is a suitable leaving group. A “suitable leaving group” is a group that is subject to nucleophilic displacement, i.e., a chemical group that is readily displaced by an incoming chemical moiety, in this case, an amino moiety of aminobutyraldehyde diethyl actetal. Suitable leaving groups are well known in the art, e.g., see, Advanced Organic Chemistry, Jerry March, 5^{th} Ed., pp. 351-357, John Wiley and Sons, N.Y. Such leaving groups include, but are not limited to, halogen and sulfonate esters. Examples of suitable leaving groups include chloro, iodo, bromo, fluoro, methanesulfonyloxy (mesyloxy), tosyloxy, triflyloxy, nitro-phenylsulfonyloxy (nosyloxy), and bromo-phenylsulfonyloxy (brosyloxy). In another embodiment, a suitable leaving group is chlorine or tosyl.

According to an alternate embodiment, the suitable leaving group may be generated in situ within the reaction medium. For example, a leaving group may be generated in situ from a precursor of that compound wherein said precursor contains a group readily replaced by said leaving group in situ.

In step S-2, INT2 is deprotected using a suitable acid to form formula INT3. HX is a suitable acid, wherein X⁻ is the anion of said suitable acid. One skilled in the art would recognize that various mineral or organic acids are suitable for achieving the deprotection. In one embodiment, a suitable mineral or organic acid includes hydrobromic acid, sulfuric acid, methanesulfonic acid and the like. In one embodiment, the suitable acid is hydrochloric acid, wherein the anion X⁻ is chloride. One of ordinary skill in the art will appreciate that X⁻ can be derived from a variety of organic and inorganic acids. In certain embodiments, X⁻ is a suitable anion. Such anions include those derived from an inorganic acid such as trifluoroacetic acid, hydrochloric acid, hydrobromic acid, phosphoric acid, sulfuric acid or perchloric acid. It is also contemplated that such anions include those derived from an organic acid such as acetic acid, oxalic acid, maleic acid, tartaric acid, citric acid, succinic acid, malonic acid, methanesulfonic acid, optionally substituted phenylsulfonic acids, sulfinic acid, optionally substituted phenylsulfinic acid, trifluoroacetic acid, trifluoromethanesulfonic (triflic) acid, optionally substituted benzoic acids, and the like. One of ordinary skill in the art will recognize that such salts are formed by other methods used in the art such as ion exchange.

For example, the general preparation of INT3 is as follows. INT1 combined with aminobutyraldehyde diethyl acetal in 2-propanol in the presence of triethylamine (TEA) at reflux temperature affords INT2. After an aqueous/organic workup (water/ethyl acetate and aqueous sodium chloride [NaCl]/ethyl acetate), treatment of crude acetal in tetrahydrofuran with aqueous HCl affords INT3 as an off-white crystalline solid. It has been surprisingly found that performing an aqueous/organic workup of INT2 at 45° C. to 50° C. prevents the precipitation of solids. It will be appreciated that INT3, although represented as the open aldehyde form in Scheme I, may be an equilibrium mixture of the aldehyde and hemiaminal tautomers shown below:

### INT3 Aldehyde-Hemiaminal Tautomers

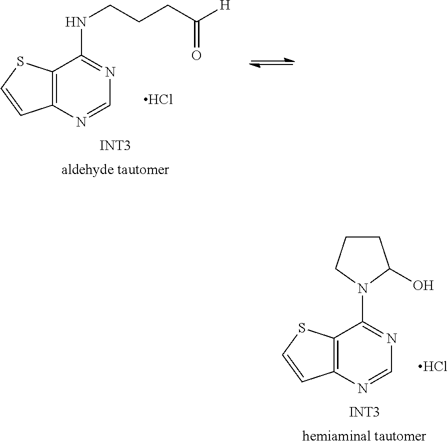

In step S-3, INT3 is combined with a suitable brominating agent to form intermediate INT4. One skilled in the art would recognize that various organic acids are suitable for achieving the bromination. In one embodiment, a suitable organic acid includes propionic acid. In one embodiment, the suitable organic acid is acetic acid. One skilled in the art would recognize that various brominating agents are appropriate for such reaction. In certain embodiments, suitable brominating agents include dibromohydantoin and N-bromosuccinimide. In one embodiment, the brominating agent is bromine. One skilled in the art would recognize that the reaction may be performed at varied temperature ranges. In one embodiment, the reaction temperature range for heating is from about 80° C. to 90° C. In one embodiment, the reaction temperature for heating is 85° C. In one embodiment, the temperature range for cooling is from about 50° C. to about 55° C.

For example, the general preparation of INT5 is as follows. INT3 is heated in acetic acid to afford a solution, cooled to 50° C. to 55° C., and then a solution of bromine is added. Heat is removed, acetone and methyl tert-butyl ether (MTBE) are added to help induce crystallization, and the resulting solid INT4 is filtered. To INT4 and thiourea is added ethanol and water and the resulting slurry heated. The reaction mixture is then concentrated to azeotropically remove water, additional ethanol is added, and then MTBE is added to help induce crystallization. INT5 is isolated as a yellow solid. It will be appreciated that INT4, although represented as the open aldehyde form in Scheme I, may be an equilibrium mixture of the aldehyde and hemiaminal tautomers as shown below:

### INT4 Aldehyde-Hemiaminal Tautomers

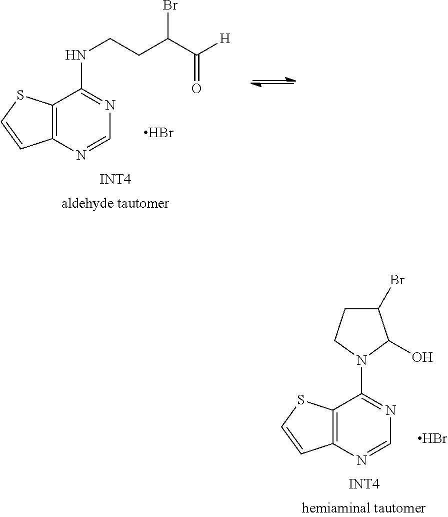

In some embodiments, the present invention provides INT4 having less than about 30%, less than about 25%, less than about 10%, less than about 5%, or less than about 1%, by weight of any of the following compounds:

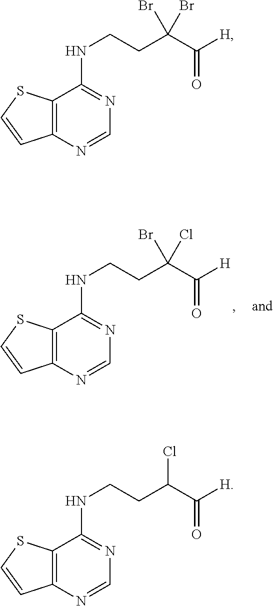

In step S-4 INT4 is coupled with thiourea to form a thiazole INT5 in a suitable solvent or solvent mixture. One skilled in the art would recognize that various solvents and/or solvent mixtures (with and without water) are appropriate for such reaction. In one embodiment, solvents and/or solvent mixtures include 100% ethanol; ethanol:water (70:30); 100% acetonitrile; acetonitrile:water (80:20). In one embodiment, the solvents and/or solvent mixture is ethanol:water (9:1). One skilled in the art would recognize that the reaction may be performed at varied temperature ranges. In one embodiment, the reaction temperature range for heating is from about 80° C. to reflux. In one embodiment, the reaction temperature is performed at reflux.

In step S-5, INT5 is coupled to 3-chlorophenyl-isocyanate to form Compound 2. One skilled in the art would recognize that various organic solvents are appropriate for such reaction. In one embodiment, such solvents include tetrahydrofuran (THF), dichloromethane (DCM), ethyl acetate, dimethylacetamide and 1,2-dichloroethane. In one embodiment, the solvent is acetonitrile. One skilled in the art would recognize that the reaction may be performed at varied temperature ranges. In one embodiment, the reaction temperature range is from about room temperature to about 80° C. In one embodiment, the reaction temperature range is from about 50° C. to about 80° C. In one embodiment, the reaction temperature range is from about 50° C. to about 55° C. One skilled in the art would recognize that various solvents and/or solvent mixtures are appropriate for reslurrying. In one embodiment, such solvents and/or solvent mixtures include 100% ethanol; acetone:methanol (50:50); ethanol:acetonitrile (50:50, or 20:80); methanol:DCM (50:50); and methanol:acetonitrile (10:90). In one embodiment, the solvent mixture is methanol:acetonitrile (1:1).

In step S-6 Compound 2 is combined with methanesulfonic acid in the presence of a suitable acid to form Compound 1 or other salt. One skilled in the art would recognize that various mineral or organic acids are suitable for achieving salt formation. In one embodiment, a suitable acid includes formic acid, propionic acid, and the like. In one embodiment, the suitable acid is acetic acid. One skilled in the art would recognize that the salt formation may be performed at varied temperature ranges. In one embodiment, the salt formation is performed at from about 60° C. to about 111° C. In one embodiment, the reaction temperature range is from about 60° C. to about 65° C. In one embodiment, the reaction temperature is about 65° C. One skilled in the art would recognize that various organic solvents are appropriate for such salt formation. In one embodiment, such solvents include methylethylketone, EtOAc, MTBE and dimethylacetamide. In one embodiment, the solvent is acetone. One skilled in the art would recognize that the salt formation may be performed at varied temperature ranges. In one embodiment, the salt formation is performed at a temperature range of from about room temperature to about 56° C. In one embodiment, the reaction performed at a temperature of about 56° C.

For example, the general preparation of Form A of Compound 1 is as follows. To a suspension of INT5 in acetonitrile is added (triethylamine) TEA and the mixture is warmed until a solution forms. 3-Chlorophenyl isocyanate is added at about 50° C. to 55° C. over 2 hours, and the mixture is then cooled and filtered. The collected solids are resuspended in hot 1:1 acetonitrile/methanol and the suspension is then cooled, filtered, and the collected solids washed with 1:1 acetonitrile/methanol to afford Compound 2. Compound 2 is dissolved in glacial acetic acid at about 60° C. to 65° C. and the solution is clarified by passing through an inline filter (10 μm).

To the resulting solution is added neat methanesulfonic acid, the mixture is cooled to about 50° C. to 55° C., and acetone is added to induce crystallization. The suspension is cooled to ambient temperature and the resulting solids collected and washed with acetone. The solids are resuspended in acetone (ACS reagent grade) and the mixture distilled to azeotropically remove water. The solids are collected, washed with acetone (low water content), and dried in a vacuum oven at elevated temperature to obtain Compound 1. In certain embodiments, use of low water content acetone in the final wash step ensures the drug substance remains in its anhydrate form. Alternatively, the hydrate form of Compound 1 can be reconverted to the anhydrate by suspension in acetone followed by azeotropic distillation.

In certain embodiments, the present invention provides Compound 1 characterized in that it has ≦410 ppm acetonitrile, ≦3,000 ppm methanol, ≦10,000 ppm acetic acid, ≦5,000 ppm acetone, or ≦5,000 ppm triethylamine present as a residual solvent.

In other embodiments, the present invention provides Compound 1 having less than about 0.5%, less than about 0.15%, or less than about 0.10%, by weight of any of the following compounds:

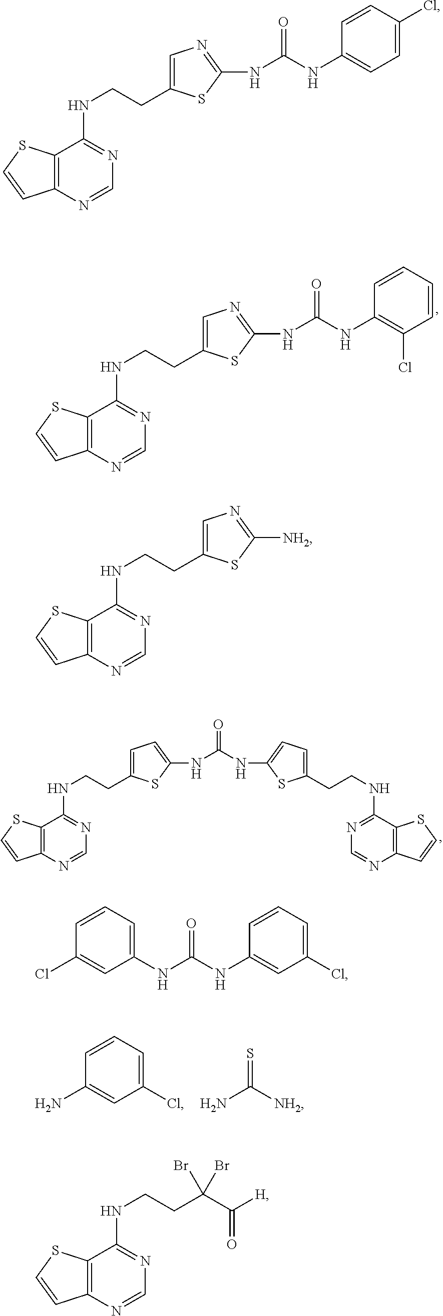

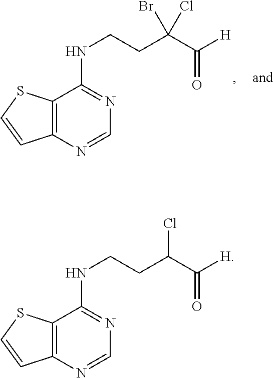

In certain embodiments the present invention provides a composition comprising Compound 1 and one or more of any of the following compounds:

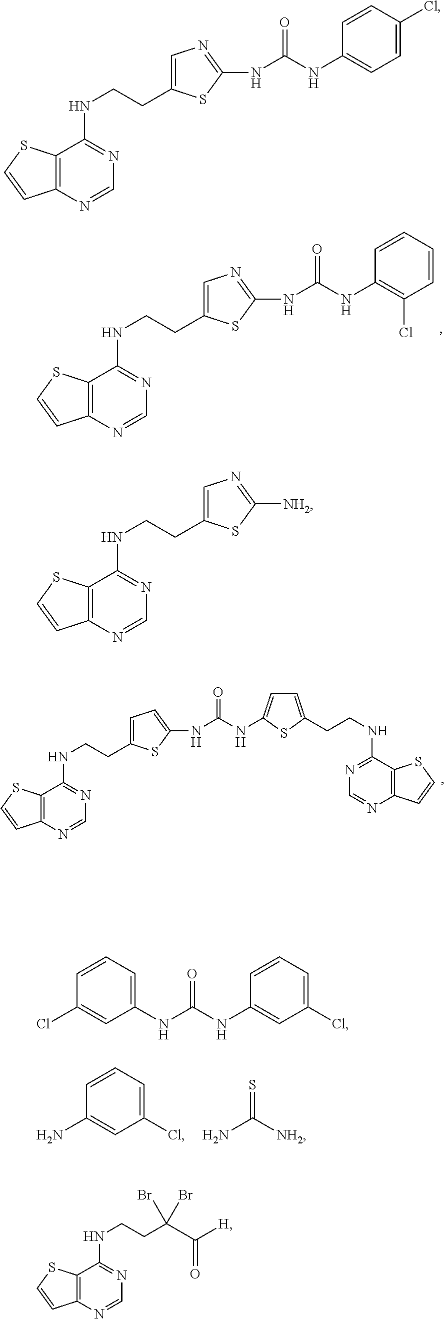

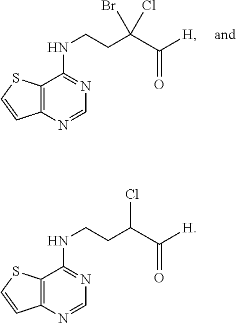

In certain embodiments, the present invention provides a method for preparing Compound 2:

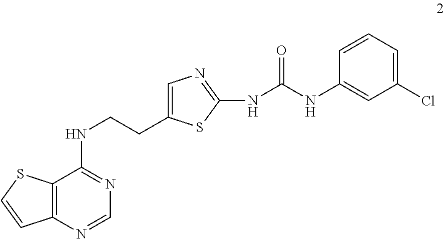

comprising the step of coupling INT5:

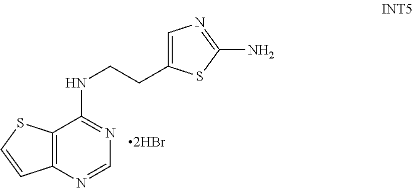

to 3-chlorophenyl-isocyanate to form Compound 2.

In certain embodiments, the present invention provides a method of preparing INT5:

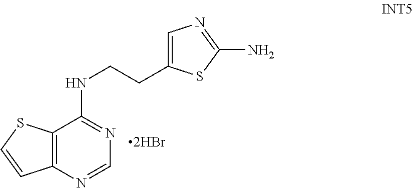

comprising the steps of:

(a) brominating INT3:

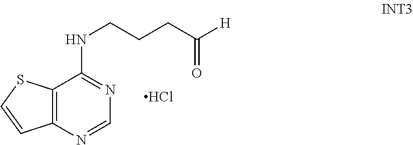

to form INT4:

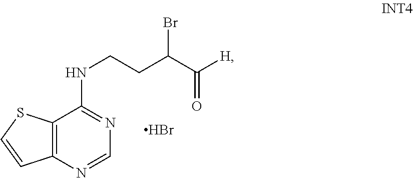

and (b) coupling INT4 with thiourea to form INT5.

In some embodiments, the present invention provides a method of preparing INT3:

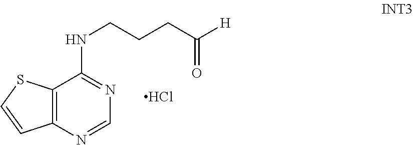

comprising the steps of:

(a) coupling INT1:

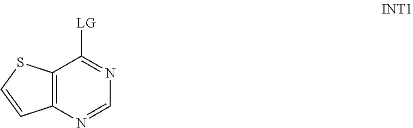

wherein LG is a suitable leaving group, with

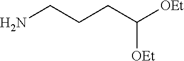

to form INT2:

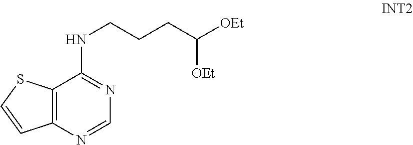

and (b) deprotecting INT2 to form INT3.

In certain embodiments, the present invention provides a method for preparing Compound 2:

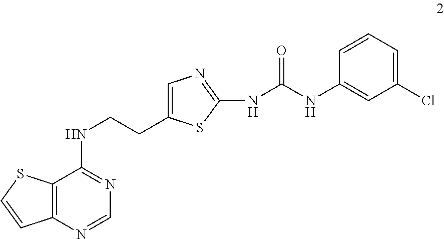

comprising the steps of:

(a) coupling INT1:

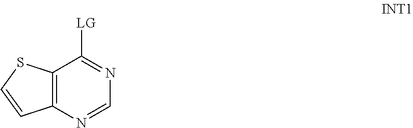

wherein LG is a suitable leaving group, with

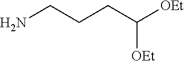

to form INT2:

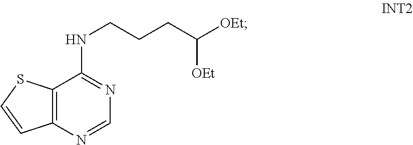

(b) deprotecting INT2 to form INT3;

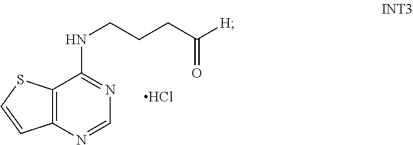

(c) brominating INT3 to form INT4:

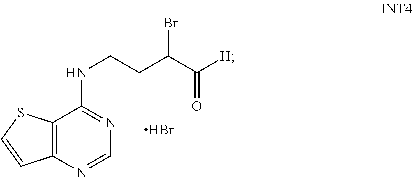

(d) coupling INT4 with thiourea to form INT5:

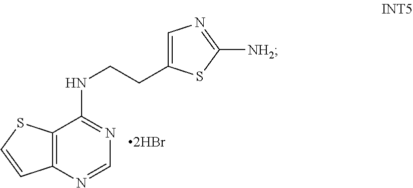

and (e) coupling INT5:

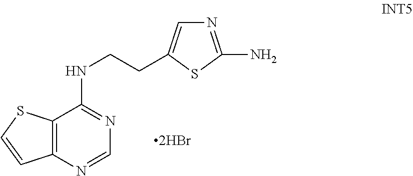

to 3-chlorophenyl-isocyanate to form Compound 2.

In some embodiments, the present invention provides a method of preparing Compound 1:

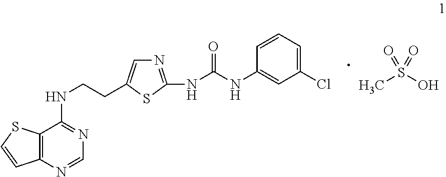

comprising the step of treating Compound 2:

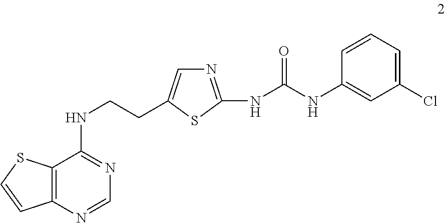

with methanesulfonic acid.

The present disclosure now being generally described, it will be more readily understood by reference to the following examples, which are included merely for purposes of illustration of certain aspects and embodiments of the present invention, and are not intended to limit the disclosure herein.

### EXEMPLIFICATION

The Aurora family of serine/threonine kinases (Aurora A, Aurora B, and Aurora C) plays a key role in cells orderly progression through mitosis. Elevated expression levels of Aurora kinases have been detected in a high percentage of melanoma, colon, breast, ovarian, gastric, and pancreatic tumors, and in a subset of these tumors the AURKA locus (20q13) is amplified. Compound 1, a novel aminothiazole-derived urea, is a selective inhibitor of Aurora kinases A, B, and C with IC₅₀ values in the low nanomolar range. Compound 1 potently inhibits cell proliferation and induces polyploidy (>4N DNA) in a diverse panel of human cancer cell lines. The pharmacodynamic effects and in vivo activity of Compound 1 were investigated in human tumor xenograft models. Compound 1 displayed potent anti-tumor activity in HCT 116 (colon), PC-3 (prostate), CALU-6 (NSCLC) and MDA-MB-231 (breast) models. Tumor growth inhibition in these xenograft models ranged from 67.5 to 96.6% on a twice-weekly administration for 3 weeks. Following Compound 1 drug administration, endoreduplication and sustained pro-apoptotic effects measured by increased PARP cleavage and Caspase activation in tumor samples were observed. Compound 1-dependent effects in surrogate tissues were also evaluated as potential biomarkers and indicators of response; inhibition of histone H3 phosphorylation was observed in bone marrow and skin epidermis obtained from mice after exposure to Compound 1 at drug levels that are efficacious and well tolerated in xenograft models. Compound 1 displays favorable pharmacokinetics with measurable drug levels sustained for more than 96 hours post-dose in the HCT 116 tumor. These drug levels were associated with prolonged inhibition of histone H3 phosphorylation, an established substrate of Aurora Kinase B. Combined, these data suggest that Compound 1 may be an effective therapeutic agent for the treatment of diverse human malignancies.

### Characterization Methods

Provided herein is an assortment of characterizing information to describe provided forms of Compound 1. It should be understood, however, that not all such information is required for one skilled in the art to determine that such particular form is present in a given composition, but that the determination of a particular form can be achieved using any portion of the characterizing information that one skilled in the art would recognize as sufficient for establishing the presence of a particular form, e.g., even a single distinguishing peak can be sufficient for one skilled in the art to appreciate that such particular form is present. United States Pharmacopeia provides additional guidance with respect to characterization of crystalline forms (see X-Ray Diffraction, <941>. United States Pharmacopeia, 31st ed. Rockville, Md.: United States Pharmacopeial Convention; 2008:372-374), which is incorporated herein by reference.

### Instrumentation

| Instrument | Vendor/Model # |
|---|---|
| Differential Scanning Calorimeter | Mettler 822^{e} DSC |
| Thermal Gravimetric Analyzer | Mettler 851^{e} SDTA/TGA |
| X-Ray Powder Diffraction System | Shimdazu XRD-6000 |
| Karl Fisher | Metrohm 756 KF Coulometer |
| Nuclear Magnetic Resonance | 500 MHz Bruker AVANCE with |
| Spectrometer | 5 mm BBO probe |
| Moisture Sorption Analysis | Hiden IGAsorp Moisture |
| | Sorption Instrument |

### Differential Scanning Calorimetry Analysis (DSC)

DSC analyses were carried out on the samples “as is”. Samples were weighed in an aluminum pan, covered with a pierced lid, and then crimped. Analysis conditions were 30° C. to 300° C. ramped at 10° C./minute.

### Thermal Gravimetric Analysis (TGA)

TGA analyses were carried out on the samples “as is”. Samples were weighed in an alumina crucible and analyzed from 30° C. to 230° C. and a ramp rate of 10° C./minute.

### X-Ray Powder Diffraction (XRPD)

Samples were analyzed “as is”. Samples were placed on Si zero-return ultra-micro sample holders and analyzed using the following conditions:

| | |
|---|---|
| X-ray tube: | Cu Kα, 40 kV, 40 mA |
| Slits | |
| Divergence Slit | 1.00 deg |
| Scatter Slit | 1.00 deg |
| Receiving Slit | 0.30 mm |
| Scanning | |
| Scan Range | 3.0-45.0 deg |
| Scan Mode | Continuous |
| Step Size | 0.04° |
| Scan Rate | 2°/min |

### Dynamic Vapor Sorption (DVS)

DVS experiments were carried out on all available forms by first drying the sample at 0% RH and 25° C. until an equilibrium weight was reached or a maximum of four hours. The sample was then subjected to an isothermal (25° C.) adsorption scan from 10 to 90% RH in steps of 10% RH. The sample was allowed to equilibrate to an asymptotic weight at each point for a maximum of four hours. Following adsorption, a desorption scan from 85 to 0% RH (at 25° C.) was run in steps of −10% RH again allowing a maximum of four hours for equilibration to an asymptotic weight. The sample was then dried for two hours at 80° C. and the resulting solid analyzed by XRPD.

### ¹H Nuclear Magnetic Resonance (¹H NMR)

Samples (2-10 mg) were dissolved in DMSO-d₆ with 0.05% tetramethylsilane (TMS) for internal reference. ¹H NMR spectra were acquired at 500 MHz using 5 mm broadband observe (¹H-X) Z gradient probe. A 30 degree pulse with 20 ppm spectral width, 1.0 s repetition rate, and 16 to 64 transients were utilized in acquiring the spectra.

### Example 1

### Preparation of Form A

For all processes, a reactor, unless otherwise stated, refers to a 72-L, unjacketed, five-neck glass reactor equipped with a mechanical stirrer [19-mm glass stir shaft, poly-tetrafluoroethylene (PTFE) stir blade], drop-bottom valve, temperature probe, and nitrogen inlet. All temperatures refer to internal temperatures unless otherwise stated. Where external cooling was applied, the reactor was placed in a steel cooling bath. For heating stages, the reactor was placed in a heating mantle and if applicable the reactor was equipped with a condenser. All table-top filter funnels were 24 inches in diameter and of polypropylene construction. All amber glass containers were fitted with a PTFE-lined closure.

### Stage 1 Preparation of INT3

To a reactor was charged INT1 (2.00 kg, 11.72 mol), 2-propanol (20 L, 10 vol), triethylamine (1.96 L, 14.07 mol), and 4-aminobutyraldehyde diethyl acetal (2.36 kg, 14.65 mol), and a portion of 2-propanol was retained to rinse the weighing containers into the reactor. The batch was heated to 75° C. and maintained at 80±5° C. for 3 hours 19 minutes prior to sampling. The analysis indicated that INT1 was 0.31% by conversion and met the specification of ≦2% by conversion. The heating was turned off, and the batch allowed to cool overnight. The resultant suspension was concentrated via a rotary evaporator (water bath at 45° C.) to a slurry and the solvent chased with ethyl acetate (EtOAc) (50 L, 25 vol). A first portion of EtOAc (3 L) was used to rinse residue from the reactor, and was subsequently added to the bulb. The remaining EtOAc (47 L) portion was added to the reactor en route to the evaporator bulb.

The batch (net 5586 g) was diluted with EtOAc (35.35 L), to a total of volume of 40 L and transferred to the reactor and heating to 50° C. was initiated. EtOAc (34 L) was preheated (50° C.) in the reactor and the batch was readily soluble. Purified water (10 L, 5 vol.) was added to the reactor stirred for 16 minutes once the batch had reached 50° C. The stirring was stopped and the phases settled and separated. Brine (10 L, 5 vol) was added to the reactor and once the batch had reheated to 50° C. (required 22 min), it was washed for 17 minutes. After the settled phases were separated, the batch was allowed to cool overnight.

The batch was concentrated via a rotary evaporator (water bath at 40° C.) to a slurry and the solvent chased with THF (50 L, 25 vol) using a similar method to that described above. The bulb was stored overnight under nitrogen at ambient temperature (net 3788 g). The batch was mobilized with THF and made up to a total of 40 L (required volume of THF was 36.5 L) and transferred to the reactor.

Hydrochloric acid (HCl) (2N, 6.25 L, 3.13 vol, 1.06 equiv) was added to the yellow solution over 1 hour 2 minutes. An initial suspension formed after approximately 1 L had been added and the addition rate was reduced, which resulted in the solids dissolving. The batch became turbid seven minutes after the addition was complete and four minutes later it was a thick yellow slurry. The stirring rate was increased to ensure that the solids mixed efficiently. HPLC analysis (TM-1486) after 4 hours 4 minutes indicated that the level of INT2 was ≦0.5% (AUC). The cream-colored slurry was stirred for 5 hours 52 minutes at ambient temperature and then filtered through a 24-inch filter funnel (polypropylene) fitted with a PTFE filter cloth (wet with 3 L of THF).

Some solids passed through the filter cloth in the initial filtration and these were re-filtered. No further solids were observed in the filtrate. Once the batch was transferred to the filter, the reactor was rinsed with THF (10 L) and the rinse transferred to the filter cake. The cake was covered with a stainless-steel filter cover and a nitrogen sweep was passed over the batch. The batch (net wet weight 5645 g) was transferred to six glass drying trays and placed into a vacuum oven (50±5° C.) and dried until the weight was constant (22 hr, 9 min). The batch was transferred to six amber glass bottles, blanketed with nitrogen, and stored at room temperature. Total yield of INT3=2.79 kg, 92% of theory.

### Stage 2 Preparation of INT5

To a reactor was charged INT3 (2500 g, 9.70 mol) and glacial acetic acid (32.5 L, 13 vol). The batch was heated to 77.5° C. over 1 hour 36 minutes when a solution formed. The batch was then cooled to 50-55° C. over 6 hours 15 minutes and when the batch reached 53° C., a solution of bromine in glacial acetic acid was added via a peristaltic pump over 45 minutes [bromine (1395 g, 8.73 mol) and glacial acetic acid (5.0 L, 2 vol)] using PTFE, polypropylene, and Pharmapure tubing. No significant exotherm or cooling was observed. The yellow solution was maintained at 50-55° C. during the HPLC analysis (TM-1493) for a total of 3 hours 56 minutes. After 1 hour 22 minutes, INT3 was above the specification of ≦4% (AUC).

An additional charge of bromine (79 g) in acetic acid (280 mL) was performed. Thirty minutes later, INT3 was 2.03% (AUC) by HPLC analysis. The heating was stopped and acetone (12.5 L, 5 vol) was added to the batch via addition funnel. MTBE (12.5 L, 5 vol) was added to the batch. The resultant yellow suspension was allowed to cool to ≦30° C. and the batch filtered using a 24-inch, table-top filter (polypropylene) fitted with a PTFE cloth. The reactor was rinsed with acetone (6.25 L, 2.5 vol) and MTBE (6.25 L, 2.5 vol) and the rinse mixed in the reactor. The rinse was applied to the cake. The yellow solid was transferred to six glass drying trays (net wet weight 3017 g) and dried in a vacuum oven at 50° C. to constant weight over 18 hours 58 minutes to give INT4 (2693 g, 73% of theory).

To a second reactor was charged INT4 (2694 g, 7.07 mol), ethanol (24.2 L, 200 Proof, 9 vol), thiourea (803 g, 10.54 mol), and purified water (2.7 L, 1 vol). The batch was heated to 78±5° C. over 1 hour 19 minutes and maintained at that temperature range for 1 hour 53 minutes. The batch was sampled for HPLC analysis and INT4 was not detected [specification was ≦1% (AUC)]. After a total of 3 hours 10 minutes, the heating was stopped and the batch cooled to <55° C.; a 10-L portion was cooled in a carboy and was concentrated ahead of the main batch. The batch was concentrated until all the batch was in the bulb (20-L) and then the ethanol rinse (26.9 L, 10 vol) was charged to the bulb. The batch was concentrated to a yellow slurry and the bulb was stored under nitrogen overnight. The batch was sampled for KF analysis which indicated a water content of 0.8% (specification ≦5%).

The batch was transferred to the second reactor in ethanol to give a total batch volume of 26.9 L (required 18 L ethanol, 200 Proof) and stirred at ambient temperature for 1 hour 22 minutes. MTBE (26.9 L, 10 vol) was added over 3 hours 12 minutes via an addition funnel (the funnel was fitted with a PTFE transfer tube to deliver the solvent between the outer side of the vortex and midway between the shaft and vessel wall). The yellow suspension was then cooled to 5-10° C. over 49 minutes and the batch was aged at this temperature range for 53 minutes (Tₘᵢₙ=6° C.). The batch was filtered through a 24-inch, table-top filter (polypropylene) fitted with a PTFE cloth and the reactor and cake were rinsed with MTBE (26.9 L, 10 vol). The residue was transferred to six glass drying trays (net wet weight 3239 g) and dried at 50° C. to constant weight which required a total time of 18 hours 58 minutes. The yellow solid was transferred to three, amber, glass jars (80 oz.) and blanketed with nitrogen. Total Yield of INT5=2783 g, 90% of theory (65% over two steps).

### Stage 3 Preparation of Compound 2

To a reactor was charged INT5 (2650 g, 6.03 mol) and acetonitrile (31.8 L, 21 vol, anhydrous) and heated to 50±5° C. with stirring over 18 minutes. Triethylamine (1.770 L, 12.67 mol, 2.1 equiv, 99.5%) was added when the temperature was 54.5° C. over 2 hours 15 minutes. An addition funnel fitted with a PTFE transfer tube was used to transfer the liquid close to the vortex. Eight minutes later, 3-chlorophenyl isocyanate (1854 g, 12.07 mol, 2.0 equiv, 99%) was added to the batch over 2 hours 3 minutes. HPLC analysis after 2 hours 12 minutes indicated INT5 was 16.2% by conversion (specification ≦4%) and 3 hours 56 minutes from the time of addition of 3-chlorophenyl isocyanate, additional 3-chlorophenyl isocyanate (370 g, 2.41 mol, 0.4 equiv) was added over 26 minutes. The batch was sampled one hour later maintaining the temperature at 50±5° C. and the level had reduced to 2.68% INT4. One hour 27 minutes from sampling, the heating was stopped and allowed to cool to <30° C. (required 5 hr 48 min).

The yellow suspension was filtered via a 24-inch, table-top filter fitted with a nylon cloth and the reactor and cake were rinsed with acetonitrile (26.5 L, 10 vol, ACS). The cake was covered with a stainless-steel filter cover under nitrogen (total filtration time 27 min). The residue was transferred back to the reactor and methanol (23.9 L, 9 vol, ACS) and acetonitrile (23.9 L, 9 vol, ACS) were added. The mixing solvents resulted in an endotherm to approximately 10° C. The batch was heated to 50±5° C. over 1 hour 2 minutes and maintained at that temperature for 6 hours 17 minutes with IPC sampling taking place after 3 hours 37 minutes. This indicated that INT5 was 0.54% (AUC) and Compound 1 was 98.3% (AUC) and the heating was discontinued. The batch was allowed to cool to <30° C. overnight.

The light yellow suspension was filtered through a 24-inch, table-top filter fitted with a nylon cloth. Acetonitrile (6.7 L, 2.5 vol, ACS) and methanol (6.7 L, 2.5 vol, ACS) were charged to the reactor and mixed to rinse the reactor. The rinse was transferred to the filter cake, and was covered with a stainless-steel filter cover and a nitrogen sweep. The light yellow residue (wet-weight 2778 g) was transferred to six glass drying trays and dried under vacuum at 50±5° C. for a total of 47 hours 9 minutes. The batch was sampled, transferred to three amber glass jars, blanketed with nitrogen and stored at room temperature. Total Yield of Compound 2=2194 g, 84% of theory.

### Preparation of Compound 1

### Final Step

To the second reactor were charged Compound 2 (2136 g and 1564 g) and acetic acid (14.8 L, 4 vol, glacial) and were heated to 50-60° C. with stirring over 37 minutes. The resultant solution was clarified into a third reactor via a transfer pump equipped with a 10-micron filter (Pall 12077) over four minutes. The batch was reheated to 60-65° C. over 19 minutes. Methanesulfonic acid (844 g, 0.228 wt) was added to the batch via an addition funnel over 1 hour 39 minutes maintaining the temperature at 60-65° C. The batch was cooled to 50-55° C. over 1 hour 22 minutes and acetone (37 L, 10 vol, clarified) was then added over 2 hours 9 minutes maintaining the temperature at 50-55° C. The batch became turbid after 14 L had been added and became a yellow suspension during 17-20 L. The heat was stopped and the batch cooled to <30° C.

The batch was filtered via a 24-inch, table-top funnel fitted with a PTFE cloth and the reactor rinsed with acetone (18.5 L, clarified) and the rinse transferred to the cake. The dense yellow residue (net wet-weight 4975 g) was transferred to six glass drying trays and dried in a vacuum oven at 55° C. to constant weight (70 hr 51 min). The batch (3985 g) was stored in the oven with the heating discontinued under vacuum until required.

To a reactor were charged Compound 1 and acetone (63 L, 17 vol, clarified). The batch was heated to 57±5° C. with stirring over 1 hour 50 minutes and distilled into a 12-L reactor whilst simultaneously adding additional remaining clarified acetone. After the addition of acetone, the batch was distilled with periodic draining of the 12-L reactor. Some of the distillate (˜8 L) possibly escaped as vapor due to the nitrogen flow used to aid distillation. The final volume was gauged by distillation to a level on the reactor. The heating was stopped, the batch cooled to <30° C. and sampled for differential scanning calorimetry (DSC) analysis. The specification was met (consistent with reference).

The batch was filtered via a 24-inch, table-top funnel fitted with a PTFE cloth and the reactor rinsed with acetone (18.5 L, J. T. Baker, low water) and the rinse transferred to the cake. The cake was covered with a stainless-steel filter funnel and a nitrogen sweep applied. The dense yellow residue (net wet-weight 4594 g) was transferred to six glass drying trays and placed into a vacuum oven, dried at 55° C. to constant weight over 70 hours 21 minutes, and then sampled for IPC analysis. The batch was maintained in the oven at 55±5° C. for 48 hours 54 minutes during the acquisition of the IPC data (total time at 55±5° C. was 119 h 15 min). The batch of Compound 1 was packaged into two containers, each consisting of two 4 mil LDPE bags, cable ties, and a desiccant bag and blanketed under nitrogen. The amount per container was 2940 g and 1010 g (3950 g, 87% of theory from Compound 2).

The XRPD and DSC patterns obtained for Form A are depicted in FIGS. 18 and 19, respectively. Characteristics of Form A are summarized in Table 13.

TABLE 13
Summary of Characteristics for Crystalline Forms A-L

| | | | TGA | | | | |
|---|---|---|---|---|---|---|---|
| | Representative | Salt Ratio | Losses | DSC Peaks | | KF Wt | |
| Form | Solvents | (¹H NMR) | (wt %) | (° C.) | DVS Result | % H₂O | Description |
| A | AcOH/ | ~1:1 | 0.0 | 229 | No form | <0.1 | Anhydrate |
| | Acetone | | | | change | | |
| B | Water slurries | ~1:1 | 2.5, 0.2 | 170 | No form | 3.0 | Mono- |
| | | | | | change, low | | hydrate |
| | | | | | hygroscopicity | | |
| C | AcOH/EtOAc | n/a | 12.6 | 164 | n/a | n/a | n/a |
| D | DMA | ~1:1 | 14-19 | ~130, ~230 | Converts to | 1.2 | DMA solvate |
| | | | | | Form B | | |
| E | Formamide | ~0.9:1 | No weight | ~163, ~219 | No form | 0.44 | Formamide |
| | | | loss below | | change, 8.0 wt | | solvate |
| | | | 140° C. | | % water uptake | | |
| | | | | | at 90% RH | | |
| F | AcOH | ~1:1 | 4.0 | 164 | n/a | n/a | n/a |
| G | MeOH | ~1:1 | 4.0 | 131, 171(x), | No form | 3.1 | Mono- |
| | | | | 212 | change, low | | hydrate |
| | | | | | hygroscopicity | | |
| H | EtOH | ~1:1 | 3.6, 3.0 | 121, 163, | Converts to | 0.50 | Ethanol |
| | | | | 170(x), | Form G | | solvate |
| | | | | 179(x), | | | |
| | | | | 221, 227 | | | |
| I | AcOH or AcOH | ~1:1 | 4.2, 4.3 | 81, 98, 164, | Converts to | 2.6 | AcOH |
| | slurry | | | 229 | Form B | | solvate or |
| | | | | | | | hydrate |
| J | DMF | ~1:1 | 6.2 | 76, 129, | Converts to | 3.4 | DMF solvate |
| | | | | 180 | Form B | | or hydrate |
| K | NMP slurry | ~1:1 | no weight | 97, 116(x) | Converts to | 0.22 | NMP solvate |
| | | | loss below | | Form B | | |
| | | | 90° C. | | | | |
| L | DMF slurry | 1:1 | 11.2 | 133, 232 | Converts to | <0.1 | DMF solvate |
| | | | | | Form B | | |

n/a: data not available.

### Example 2

### Preparation of Form B

Compound 1 (Form A, 291 mg) was dissolved in DMF (3 mL) at 55° C. followed by hot filtration and addition of THF (29 mL). This mixture was placed in the refrigerator for fast cooling and held at 4° C. for 16 hours. The resulting solids were isolated by filtration, dried in vacuo (room temperature, 30 mm Hg) to afford Form B of Compound 1 (290.8 mg). The XRPD and DSC patterns obtained for Form B are depicted in FIGS. 20 and 21, respectively. Characteristics of Form B are summarized in Table 13.

### Example 3

### Preparation of Form C

Compound 2 (500 mg) in acetic acid (5 mL) was heated to 55° C. and then a solution of methanesulfonic acid (1.05 equivalents) in acetic acid (2 mL) was added. The solution was cooled to 42° C. and then EtOAc (10 mL) was added, resulting in the formation of solids. The mixture was cooled to room temperature over 1 hour, filtered, and the solids washed with ethyl acetate (10 mL) then dried in a vacuum oven at 50° C. to afford Form C of Compound 1 (650 mg). The XRPD and DSC patterns obtained for Form C are depicted in FIGS. 22 and 23, respectively. Characteristics of Form C are summarized in Table 13.

### Example 4

### Preparation of Form D

Compound 1 (Form A, 204.3 mg) was weighed out into vial and dimethylacetamide (1.3 mL) was added until the material went into solution at 55° C. The resulting solution was then clarified by hot filtration through a syringe filter (Millipore Millex-FH). After filtration, the vial was slowly cooled to room temperature at a rate of 20° C. per hour and further stirred at room temperature for 16 hours. The resulting solids were collected by vacuum filtration and dried (in vacuo, room temperature, 30 inches Hg) to afford Form D of Compound 1 (219.0 mg). The XRPD and DSC patterns obtained for Form D are depicted in FIGS. 24 and 25, respectively. Characteristics of Form D are summarized in Table 13.

### Example 5

### Preparation of Form E

Compound 1 (Form A, 361 mg) was dissolved in formamide (4 mL) at 55° C. and held at this temperature with stirring for approximately one hour. After the initial dissolution, a precipitate was observed to form at 55° C. within five minutes. The resulting slurry was slowly cooled to room temperature at a rate of 20° C. per hour and further held at room temperature for 16 hours. The resulting solids were isolated by filtration, dried (in vacuo, room temperature, 30 mm Hg) to afford Form E of Compound 1 (305.2 mg). The XRPD, and DSC patterns obtained for Form E are depicted in FIGS. 26 and 27, respectively. Characteristics of Form E are summarized in Table 13.

### Example 6

### Preparation of Form F

Compound 1 (Form A, 30 mg) was weighed out into a vial and acetic acid (0.2 mL) was added until the material went into solution at 55° C. The obtained solution was then slowly cooled to room temperature at a rate of 20° C. per hour and the resulting slurry further stirred at room temperature for 16 hours. The obtained solids were isolated by filtration, dried (in vacuo, room temperature, 30 mm Hg) to afford Form F of Compound 1 (16.7 mg). The XRPD and DSC patterns obtained for Form F are depicted in FIGS. 28 and 29, respectively. Characteristics of Form F are summarized in Table 13.

### Example 7

### Preparation of Form G

Compound 1 (Form A, 153.5 mg) was weighed out into a vial and methanol (3.2 mL) was added to form a slurry. The slurry was stirred at 55° C. for one hour then slowly cooled to room temperature at a rate of 20° C. per hour and further held at this temperature for 16 hours. The resulting solids were collected by vacuum filtration and dried (in vacuo, room temperature, 30 inches Hg) to afford Form G of Compound 1 (145 mg). The XRPD and DSC patterns obtained for Form G are depicted in FIGS. 30 and 31, respectively. Characteristics of Form G are summarized in Table 13.

### Example 8

### Preparation of Form H

Compound 1 (Form A, 193.4 mg) was weighed out into a vial and ethanol (3.2 mL) was added to form slurry. The slurry was stirred at 55° C. for one hour then slowly cooled to room temperature at a rate of 20° C. per hour and further held at this temperature for 16 hours. The resulting solids were collected by vacuum filtration and dried (in vacuo, room temperature, 30 inches Hg) to afford Form H of Compound 1 (187.2 mg). The XRPD and DSC patterns obtained for Form H are depicted in FIGS. 32 and 33, respectively. Characteristics of Form H are summarized in Table 13.

### Example 9

### Preparation of Form I

Compound 1 (Form A, 300 mg) was dissolved in acetic acid (2 mL) at 55° C., stirred at this temperature for approximately one hour and slowly cooled to room temperature at a rate of 20° C. per hour. The obtained slurry was then stirred at room temperature for 16 hours. The solids were isolated by filtration, dried (in vacuo, room temperature, 30 mm Hg) to afford Form I of Compound 1 (268 mg). The XRPD and DSC patterns obtained for Form I are depicted in FIGS. 34 and 35, respectively. Characteristics of Form I are summarized in Table 13.

### Example 10

### Preparation of Form J

Compound 1 (Form A, 192.7 mg) was weighed out into a vial and DMF (1.4 mL) was added until the material went into solution at 55° C. The resulting solution was filtered hot through a syringe filter (Millipore Millex-FH) then slowly cooled to room temperature at the rate of 20° C. per hour and further stirred at room temperature for 16 hours. The resulting solids were collected by vacuum filtration and dried (in vacuo, room temperature, 30 inches Hg) to afford Form J of Compound 1 (120 mg). The XRPD and DSC patterns obtained for Form J are depicted in FIGS. 36 and 37, respectively. Characteristics of Form J are summarized in Table 13.

### Example 11

### Preparation of Form K

Compound 1 (Form A, 711 mg) was reslurried in N-methylpyrrolidine (1.5 mL) at room temperature for 19 hours. The resulting solids were isolated by filtration and dried (in vacuo, room temperature, 30 inches Hg) to afford Form K of Compound 1 (657 mg). The XRPD and DSC patterns obtained for Form K are depicted in FIGS. 38 and 39, respectively. Characteristics of Form K are summarized in Table 13.

### Example 12

### Preparation of Form L

Compound 1 (Form A, 500 mg) was reslurried in DMF (2.5 mL) at 40° C. for one week. The resulting solids were isolated by filtration and dried (in vacuo, room temperature, 30 inches Hg) to afford Form L of Compound 1 (405 mg). The XRPD and DSC patterns obtained for Form K are depicted in FIGS. 40 and 41, respectively. Characteristics of Form L are summarized in Table 13.

### Example 13

### Solubility Screen

A solubility study of Compound 1 Form A in various solvents was executed to determine its solubility in various solvents. The results are summarized in Table 14. Compound 1 Form A was placed in vials and the chosen solvents were dispensed in 100 μL portions into the corresponding vials. The solvents were chosen based on differences in polarity and functionality and on their classification according to the International Conference on Harmonization (ICH), with preference given to class II and class III solvents. After each addition of solvent, the vials were visually inspected to assess dissolution and further heated to 55° C. to ensure dissolution.

Compound 1 Form A is soluble in DMF, NMP, DMA, formamide, AcOH and is sparingly soluble in methanol and ethanol. Compound 1 Form A showed poor solubility in THF, EtOAc, MeCN, acetone, MEK, IPA, water, dioxane, MTBE, IPAc, heptane, CH₂Cl₂ and toluene.

TABLE 14
Approximate Solubility of Compound 1 Form A

| | Material | Solvent | | | | |
|---|---|---|---|---|---|---|
| | Amount | Amount | Conc. | | | ICH |
| Solvent | (mg) | (mL) | (mg/mL) | Temp | Soluble | Class |
| DMF | 0.9 | 0.10 | >9.00 | RT | Yes | II |
| NMP | 1.4 | 0.10 | >14.00 | RT | Yes | II |
| DMA | 2.8 | 0.10 | >28.00 | RT | Yes | II |
| Formamide | 1.6 | 0.10 | >16.00 | RT | Yes | |
| AcOH | 1.7 | 0.10 | >17.00 | RT | Yes | III |
| MeOH | 2.1 | 3.00 | ~0.70 | 55 | Yes | II |
| THF | 1.9 | 6.00 | <0.32 | 55 | No | II |
| EtOAc | 1.5 | 6.00 | <0.25 | 55 | No | III |
| MeCN | 2.5 | 6.00 | <0.42 | 55 | No | II |
| Acetone | 2.2 | 6.00 | <0.37 | 55 | No | III |
| MEK | 2.9 | 6.00 | <0.48 | 55 | No | III |
| IPA | 1.8 | 6.00 | <0.30 | 55 | Partially | III |
| EtOH | 1.9 | 3.00 | ~0.63 | 55 | Yes | III |
| Water | 1.7 | 6.00 | <0.28 | 55 | No | III |
| Dioxane | 1.7 | 6.00 | <0.28 | 55 | No | II |
| MTBE | 1.2 | 6.00 | <0.20 | 55 | No | III |
| IPAc | 2.4 | 6.00 | <0.40 | 55 | No | III |
| Heptane | 2.6 | 6.00 | <0.43 | 55 | No | III |
| DCM | 2.6 | 6.00 | <0.43 | 55 | No | II |
| Toluene | 2.0 | 6.00 | <0.33 | 55 | No | II |

### Example 14

### Single Solvent Recrystallization/Reslurry with Slow Cooling

Based on the initial solubility study, seven solvents were selected for the slow cooling crystallization: DMF, NMP, DMA, formamide, AcOH, methanol, and ethanol. Compound 1 (approximately 30 mg) was weighed out into vials. and solvent was added until the material went into solution at elevated temperature (this applies to the primary solvents DMF, NMP, DMA, formamide, acetic acid); other solvents were added to form slurries and stirred at 55° C. for approximately two hours. The vials were then slowly cooled to room temperature at a rate of 20° C./h and further stirred at room temperature for 16 hours. Table 15 shows all experimental details. Samples 15 and 16 using MeOH and EtOH respectively were filtered hot to remove some residual insoluble material and then were also slowly cooled to room temperature. After the cooling process, precipitates were isolated by filtration. Sample 2 did not produce any solid and was therefore concentrated under a gentle nitrogen flow overnight. The recovered materials from all experiments were dried in vacuo at room temperature and 30 inches Hg.

Forms D, E, F, G, and H were obtained from single solvent recrystallizations from DMA, formamide, AcOH, MeOH, and EtOH, respectively. The unique XRPD patterns for these forms are shown in FIGS. 24, 26, 28, 30, and 32, respectively.

The single solvent recrystallization/reslurry from THF, EtOAc, MeCN, acetone, MEK, and IPA for Compound 1 produced samples showing XRPD patterns consistent with Form A. These samples were the same form as the starting material most likely due to the poor solubility of Compound 1 in these solvents.

Form B was produced from water. Form B was also produced from DMF and NMP, indicating that the residual water in these solvents is enough to trigger a form conversion to the hydrate.

TABLE 15
Single Solvent Recrystallization/Reslurry using a Slow Cooling Procedure

| | | MSA Salt | Solv. | | | | |
|---|---|---|---|---|---|---|---|
| | Boiling | Amount | Amt | Conc. | Temp | | |
| Solvent | Point | (mg) | (mL) | (mg/mL) | (° C.) | Precipit. | Form |
| DMF | 153 | 26.2 | 0.20 | 131.00 | 55 | Yes | B |
| NMP | 82/10 mm | 39.1 | 0.20 | 195.50 | 55 | No/evap | B |
| DMA | 165 | 29.9 | 0.20 | 149.50 | 55 | Yes | D |
| | | | | | | (55° C.) | |
| Formamide | 210 | 36.1 | 0.40 | 90.25 | 55/100 | Yes | E |
| | | | | | | (55° C.) | |
| AcOH | 117 | 30.0 | 0.20 | 150.00 | 55 | Yes | F |
| MeOH | 64 | 24.3 | 0.50 | 48.60 | 55 | Slurry | G |
| THF | 65 | 28.7 | 0.50 | 57.40 | 55 | Slurry | A |
| EtOAc | 76 | 27.5 | 0.50 | 55.00 | 55 | Slurry | A |
| MeCN | 81 | 30.6 | 0.50 | 61.20 | 55 | Slurry | A |
| Acetone | 56 | 32.8 | 0.50 | 65.60 | 55 | Slurry | A |
| MEK | 80 | 22.3 | 0.50 | 44.60 | 55 | Slurry | A |
| IPA | 82 | 26.5 | 0.50 | 53.00 | 55 | Slurry | A |
| EtOH | 78 | 26.3 | 0.50 | 52.60 | 55 | Slurry | H |
| Water | 100 | 21.9 | 0.50 | 43.80 | 55 | Slurry | B |
| MeOH* | 64 | 32.5 | 7.0 | 4.64 | 62 | Yes | G |
| EtOH* | 78 | 30.8 | 7.0 | 4.40 | 75 | Yes | H |
| | | | | | | (42° C.) | |

*Samples were filtered hot

### Example 15

### Binary Solvent Recrystallizations

Binary solvent recrystallizations of Compound 1 were performed using five primary solvents (DMF, NMP, DMA, formamide, and AcOH) and eight co-solvents (MeOH, EtOH, THF, EtOAc, MeCN, acetone, MEK, and IPA) with fast and slow cooling profiles. Tables 16-28 provide detailed information for these sets of experiments.

### Fast Cooling Profile

Compound 1 (approximately 30 mg) was weighed out into vials, and primary solvent was added until the material went into solution at elevated temperature. After hot filtration, the anti-solvent was added portionwise until the solution became turbid or the vial was full. The vials were then placed in a refrigerator and held at 4° C. for 16 hours. After the cooling process, precipitates were isolated by filtration, and dried in vacuo at room temperature and 30 inches Hg. The vials without solids were evaporated to dryness using a gentle stream of nitrogen. The solids obtained were also dried in vacuo at ambient temperature and 30 inches Hg.

### Slow Cooling Profile

Compound 1 (approximately 30 mg of Form A) was weighed into vials, and primary solvent was added until the material went into solution at elevated temperature. After a hot filtration, the anti-solvent was added portionwise until the solution became turbid or the vial was full, consistent with the fast cooling experiments. The vials were then slowly cooled to room temperature at a rate of 20° C./h from 55° C. After the cooling process, precipitates were isolated by filtration, and dried in vacuo at ambient temperature and 30 inches Hg. The vials without solids were evaporated to dryness or until a precipitate was formed using a gentle stream of nitrogen. The resultant solids were also dried in vacuo at room temperature and 30 inches Hg. All solids obtained were analyzed by XRPD to determine the physical form of the obtained material.

### Formamide as Primary Solvent

As was observed during single solvent crystallizations, a minimum amount of formamide (0.3 mL) dissolved the starting material at 55° C. and very quickly produced a precipitate, which was found to be Form E. In order to avoid premature crystallization of the material in this experiment, an additional amount of formamide was added at 100° C. before the addition of the anti-solvent, Table 16. After a hot filtration, the anti-solvent was added but the solution did not become turbid, even after reaching the maximum volume allowable by the size of the crystallization vials (8 mL). The vials were then placed in a refrigerator (4° C.) and held at this temperature for 16 hours, during which time no precipitation was observed. The solutions were transferred to larger vials (20 mL) and another 13 mL of the chosen anti-solvents were added to each vial. The resulting solutions were further held at 4° C. for 24 hours, during which time no precipitate was generated. All vials were evaporated to dryness using a gentle stream of nitrogen. The resulting solids were dried in vacuo at room temperature and 30 inches Hg and analyzed by XRPD. These forms were observed to be unique compared to single solvent crystallization but subsequent analysis by NMR indicated that the material was consistent with the free base and not the mesylate salt.

TABLE 16
Binary Solvent Recrystallizations of Compound 1 using Formamide
as a Primary Solvent* and a Fast Cooling Procedure

| | | | | Appearance | | | |
|---|---|---|---|---|---|---|---|
| Cmpd 1 | Formamide | | Antisolvent | After Co- | | | |
| Amount | Amount | Anti- | Amount | solvent | | | Recovery |
| (mg) | (mL) | solvent | (mL) | Addition | Precipitate | Form | (mg) |
| 28.4 | 3.50 | MeOH | 4 + 13 | Clear | evap to ppt | FB | 13.7 |
| 25.0 | 2.90 | THF | 4 + 13 | Clear | evap to ppt | FB | 11.8 |
| 29.6 | 3.50 | EtOAc | 4 + 13 | Clear | evap to ppt | FB | 14.8 |
| 25.0 | 2.90 | MeCN | 4 + 13 | Clear | evap to ppt | FB | 11.2 |
| 24.9 | 2.90 | Acetone | 4 + 13 | Clear | evap to ppt | FB | 11.2 |
| 30.0 | 3.50 | MEK | 4 + 13 | Clear | evap to ppt | FB | 12 |
| 23.9 | 2.90 | IPA | 4 + 13 | Clear | evap to ppt | FB | 10.3 |
| 29.7 | 3.50 | EtOH | 4 + 13 | Clear | evap to ppt | FB | 12.9 |

*Solids were dissolved at 100° C.
FB—Free base, not a salt

Slow cooling procedure of binary solvent crystallizations with formamide as primary solvent afforded mostly Form E material with the exceptions of MeOH as anti-solvent which produced Form G, MeCN and IPA produced Form A and EtOH produced Form H. Table 17 provides a summary of the detailed information about this experiment.

TABLE 17
Binary Solvent Crystallizations of Compound 1 using Formamide
as a Primary Solvent and a Slow Cooling Procedure

| | | | | Appearance | | | |
|---|---|---|---|---|---|---|---|
| Cmpd 1 | Formamide | | Antisolvent | After Co- | | | |
| Amount | Amount | Anti- | Amount | solvent | | | Recovery |
| (mg) | (mL) | solvent | (mL) | Addition | Precipitate | Form | (mg) |
| 26.6 | 0.3 | MeOH | 7.00 | Clear | Yes | G | 11.3 |
| 25.9 | 0.3 | THF | 1.00 | Turbid | Yes | E | 15.8 |
| 29.5 | 0.3 | EtOAc | 0.47 | Turbid | Yes | E | 18 |
| 25.4 | 0.3 | MeCN | 1.00 | Turbid | Yes | A | 14.1 |
| 28.8 | 0.3 | Acetone | 1.00 | Turbid | Yes | E | 18.7 |
| 29.0 | 0.3 | MEK | 1.00 | Turbid | Yes | E | 16 |
| 25.9 | 0.3 | IPA | 7.00 | Clear | Yes | A | 15.6 |
| 28.3 | 0.3 | EtOH | 7.00 | Clear | Yes | H | 14.6 |
| 27.7 | 0.3 | n/a | n/a | n/a | Yes | E | 12.2 |

### DMF as Primary Solvent

Both fast cooling (Table 18) and slow cooling (Table 19) experiments using DMF as a primary solvent showed that MeOH (as anti-solvent) produces Form G, and EtOH produced Form H in slow cooling and Form B in fast cooling procedures. All other anti-solvents produced Form A in slow cooling procedure and Forms A or B in fast cooling procedures (see Table 16 and Table 21).

TABLE 18
Binary Solvent Recrystallizations of Compound 1 using
DMF as a Primary Solvent and a Fast Cooling Procedure

| | | | | Appearance | | | |
|---|---|---|---|---|---|---|---|
| Cmpd 1 | DMF | | Antisolvent | After Co- | | | |
| Amount | Amount | Anti- | Amount | solvent | | | Recovery |
| (mg) | (mL) | solvent | (mL) | Addition | Precipitate | Form | (mg) |
| 26.5 | 0.3 | MeOH | 6.0 | Clear | Small | G | 12.5 |
| 29.1 | 0.3 | THF | 2.9 | Turbid | Yes | B | 18.8 |
| 24.4 | 0.3 | EtOAc | 0.6 | Turbid | Yes | B+ | 14.0 |
| 30.7 | 0.3 | MeCN | 1.0 | Turbid | Yes | A | 21.2 |
| 26.7 | 0.3 | Acetone | 1.0 | Turbid | Yes | A | 15.1 |
| 25.0 | 0.3 | MEK | 1.5 | Turbid | Yes | A | 16.6 |
| 27.7 | 0.3 | IPA | 6.0 | Clear | Small | B | 15.5 |
| 28.2 | 0.3 | EtOH | 6.0 | Clear | Small | B+ | 17.1 |

B+: Form B with extra diffraction peaks

TABLE 19
Binary Solvent Recrystallizations of Compound 1 using
DMF as a Primary Solvent and a Slow Cooling Procedure

| | | | | Appearance | | | |
|---|---|---|---|---|---|---|---|
| Cmpd 1 | DMF | | Antisolvent | After Co- | | | |
| Amount | Amount | Anti- | Amount | solvent | | | Recovery |
| (mg) | (mL) | solvent | (mL) | Addition | Precipitate | Form | (mg) |
| 28.4 | 0.3 | MeOH | 7.0 | Clear | Yes | G | 15.2 |
| 28.3 | 0.3 | THF | 2.9 | Turbid | Yes | A | 20 |
| 28.5 | 0.3 | EtOAc | 0.6 | Turbid | Yes | A | 19.9 |
| 31.1 | 0.3 | MeCN | 1.0 | Turbid | Yes | A | 21.6 |
| 30.5 | 0.3 | Acetone | 1.0 | Turbid | Yes | A | 20.8 |
| 29.4 | 0.3 | MEK | 1.0 | Turbid | Yes | A | 20.3 |
| 25.2 | 0.3 | IPA | 7.0 | Clear | Yes | A | 17 |
| 31.4 | 0.3 | EtOH | 7.0 | Clear | Yes | H | 17.6 |

### DMA as Primary Solvent

It was observed that most of the solvent mixtures in the binary solvent experiments with DMA as a primary solvent produced Form D with the exceptions of MeOH (Form G) and EtOH (Form H), and occasionally Forms A and B were also obtained (Table 20 and Table 21).

For the samples in Table 23, it was noted that after the dissolution of the starting material in DMA and stirring at 55° C. for 5-10 minutes, a very fine precipitate was formed in samples 1-3 and 5-8. In samples 1-3 and 5-6, this material went through the syringe filter (Millex-HV) during filtration. In the last two samples 7 and 8, this material was caught in the syringe filter.

TABLE 20
Binary Solvent Recrystallizations of Compound 1 using DMA as a
Primary Solvent and a Fast Cooling Procedure

| | | | | Appearance | | | Re- |
|---|---|---|---|---|---|---|---|
| Cmpd 1 | DMA | | | After Co- | Pre- | | cov- |
| Amount | Amount | Anti- | Amount | solvent | cip- | | ery |
| (mg) | (mL) | solvent | (mL) | Addition | itate | Form | (mg) |
| 30.1 | 0.3 | MeOH | 3.00 | Turbid | Yes | G | 12.8 |
| 28.5 | 0.3 | THF | 1.00 | Turbid | Yes | D | 16.7 |
| 28.0 | 0.3 | EtOAc | 0.60 | Turbid | Yes | D | 16.2 |
| 26.5 | 0.3 | MeCN | 0.85 | Turbid | Yes | B | 14.8 |
| 30.2 | 0.3 | Acetone | 0.55 | Turbid | Yes | D | 18.2 |
| 27.5 | 0.3 | MEK | 0.65 | Turbid | Yes | D | 18.9 |
| 28.0 | 0.3 | IPA | 6.00 | Turbid | Yes | B | 16.8 |
| 24.9 | 0.3 | EtOH | 6.00 | Clear | Yes | H | 11.0 |

TABLE 21
Binary Solvent Crystallizations of Compound 1 using DMA as a
Primary Solvent and a Slow Cooling Procedure

| | | | | Appearance | | | |
|---|---|---|---|---|---|---|---|
| Cmpd 1 | DMA | | Antisolvent | After Co- | | | |
| Amount | Amount | Anti- | Amount | solvent | | | Recovery |
| (mg) | (mL) | solvent | (mL) | Addition | Precipitate | Form | (mg) |
| 28.6 | 0.3 | MeOH | 7.00 | Clear | Yes | G | 11.6 |
| 29.6 | 0.3 | THF | 1.00 | Turbid | Yes | D | 20.5 |
| 27.8 | 0.3 | EtOAc | 0.60 | Turbid | Yes | B+ | 19 |
| 25.3 | 0.3 | MeCN | 0.70 | Turbid | Yes | D | 14.6 |
| 31.2 | 0.3 | Acetone | 0.65 | Turbid | Yes | D | 25.2 |
| 31.5 | 0.3 | MEK | 0.65 | Turbid | Yes | D | 24.8 |
| 31.3 | 0.3 | IPA | 7.00 | Clear | Yes | A | 20.5 |
| 31.7 | 0.3 | EtOH | 7.00 | Clear | Yes | H | 15.8 |

B+: Form B with extra diffraction peaks

Fast cooling binary solvent crystallizations with DMA as the primary solvent were re-evaluated using a different crystallization technique. Each sample was dissolved in DMA without extra stirring at 55° C. for 5-10 minutes as was done before. Upon dissolution the solution was hot filtered through a syringe filter (Millex-FH) followed by fast addition of the anti-solvent. This was done to avoid any premature precipitation in the DMA. Table 22 summarizes the experimental details. The results were similar to the first experiment, with the exception that most of the solvents produced mixtures of Forms B and D instead of pure Form D.

TABLE 22
Binary Solvent Crystallizations of Compound 1 using DMA as a Primary Solvent
and a Fast Cooling Procedure

| | | | | Appearance | | | |
|---|---|---|---|---|---|---|---|
| Cmpd 1 | DMA | | Antisolvent | After Co- | | | |
| Amount | Amount | Anti- | Amount | solvent | | | Recovery |
| (mg) | (mL) | solvent | (mL) | Addition | Precipitate | Form | (mg) |
| 28.3 | 0.3 | MeOH | 7.00 | Clear | Yes | G | 15.3 |
| 28.6 | 0.3 | THF | 1.00 | Turbid | Yes | D | 22.9 |
| 28.8 | 0.3 | EtOAc | 0.47 | Turbid | Yes | B + D | 20.6 |
| 28.4 | 0.3 | MeCN | 0.93 | Turbid | Yes | B + D | 20.3 |
| 26.1 | 0.3 | Acetone | 1.00 | Turbid | Yes | B + D | 18.6 |
| 26.2 | 0.3 | MEK | 1.00 | Turbid | Yes | B + D | 18.7 |
| 27.1 | 0.3 | IPA | 7.00 | Clear | Yes | B | 14.1 |
| 26.5 | 0.3 | EtOH | 7.00 | Clear | Yes | H | 12.2 |
| 26.5 | 0.3 | n/a | n/a | n/a | Yes | B | 18.3 |

### NMP as Primary Solvent

Fast cooling binary solvent experiments (Table 23) using NMP as a primary solvent produced mainly Form B compared to slow cooling (Table 24) experiments which provided mostly Form A. Methanol and ethanol produced Forms G and H respectively in both fast and slow cooling experiments.

TABLE 23
Binary Solvent Recrystallizations of Compound 1 using NMP as a Primary Solvent
and a Fast Cooling Procedure

| | | | | Appearance | | | |
|---|---|---|---|---|---|---|---|
| Cmpd 1 | NMP | | Antisolvent | After Co- | | | |
| Amount | Amount | Anti- | Amount | solvent | | | Recovery |
| (mg) | (mL) | solvent | (mL) | Addition | Precipitate | Form | (mg) |
| 26.1 | 0.3 | MeOH | 6.0 | Clear | Small | G | 10.0 |
| 30.2 | 0.3 | THF | 3.9 | Turbid | Yes | B | 17.0 |
| 28.3 | 0.3 | EtOAc | 0.6 | Turbid | Yes | B+ | 16.4 |
| 30.1 | 0.3 | MeCN | 2.0 | Turbid | Yes | A | 16.0 |
| 28.1 | 0.3 | Acetone | 1.9 | Turbid | Yes | B | 13.0 |
| 26.3 | 0.3 | MEK | 2.5 | Turbid | Yes | A | 12.4 |
| 29.3 | 0.3 | IPA | 6.0 | Clear | Small | B | 15.7 |
| 27.2 | 0.3 | EtOH | 6.0 | Clear | Small | B+ | 12.3 |

B+: Form B with extra diffraction peaks

TABLE 24
Binary Solvent Crystallizations of Compound 1 using NMP as a Primary Solvent
and a Slow Cooling Procedure

| | | | | Appearance | | | |
|---|---|---|---|---|---|---|---|
| Cmpd 1 | NMP | | Antisolvent | After Co- | | | |
| Amount | Amount | Anti- | Amount | solvent | | | Recovery |
| (mg) | (mL) | solvent | (mL) | Addition | Precipitate | Form | (mg) |
| 26.3 | 0.3 | MeOH | 7.0 | Clear | Yes | G | 9.3 |
| 25.1 | 0.3 | THF | 5.0 | Turbid | Yes | A | 13.3 |
| 26.4 | 0.3 | EtOAc | 0.6 | Turbid | Yes | B+ | 18.3 |
| 25.0 | 0.3 | MeCN | 2.0 | Turbid | Yes | A | 12.4 |
| 28.3 | 0.3 | Acetone | 1.95 | Turbid | Yes | A | 15.2 |
| 30.2 | 0.3 | MEK | 2.0 | Turbid | Yes | A | 16.2 |
| 30.0 | 0.3 | IPA | 7.0 | Clear | Yes | A | 14.3 |
| 26.9 | 0.3 | EtOH | 7.0 | Clear | Yes | H | 10.2 |

B+: Form B with extra diffraction peaks

### AcOH as Primary Solvent

Most of the anti-solvents provided Form A in both fast cooling and slow cooling experiments using AcOH as primary solvent. Tables 25 and Table 26 summarize experimental details and results.

TABLE 25
Binary Solvent Recrystallizations of Compound 1 using AcOH as a Primary Solvent
and a Fast Cooling Procedure

| | | | | Appearance | | | |
|---|---|---|---|---|---|---|---|
| Cmpd 1 | AcOH | | Antisolvent | After Co- | | | |
| Amount | Amount | Anti- | Amount | solvent | | | Recovery |
| (mg) | (mL) | solvent | (mL) | Addition | Precipitate | Form | (mg) |
| 30.8 | 0.3 | MeOH | 6.00 | Clear | Yes | G | 14.4 |
| 30.9 | 0.3 | THF | 0.65 | Turbid | Yes | A | 13.5 |
| 29.6 | 0.3 | EtOAc | 0.40 | Turbid | Yes | B+ | 17.4 |
| 30.3 | 0.3 | MeCN | 3.00 | Turbid | Yes | A | 19.1 |
| 31.1 | 0.3 | Acetone | 2.00 | Turbid | Yes | A | 18.6 |
| 26.9 | 0.3 | MEK | 2.60 | Turbid | Yes | A | 15.8 |
| 27.1 | 0.3 | IPA | 6.00 | Clear | Yes | B | 19.1 |
| 30.2 | 0.3 | EtOH | 2.00 | Turbid | Yes | H | 19.6 |

B+: Form B with extra diffraction peaks

TABLE 26
Binary Solvent Crystallizations of Compound 1 using AcOH as a Primary Solvent and
a Slow Cooling Procedure

| | | | | Appearance | | | |
|---|---|---|---|---|---|---|---|
| Cmpd 1 | AcOH | | Antisolvent | After Co- | | | |
| Amount | Amount | Anti- | Amount | solvent | | | Recovery |
| (mg) | (mL) | solvent | (mL) | Addition | Precipitate | Form | (mg) |
| 25.4 | 0.3 | MeOH | 7.00 | Clear | Yes | G | 12.9 |
| 27.0 | 0.3 | THF | 0.65 | Turbid | Yes | A | 14.4 |
| 26.3 | 0.3 | EtOAc | 0.40 | Turbid | Yes | E | 16.8 |
| 26.9 | 0.3 | MeCN | 4.00 | Turbid | Yes | A | 14.3 |
| 28.3 | 0.3 | Acetone | 2.00 | Turbid | Yes | A | 17.8 |
| 24.7 | 0.3 | MEK | 2.60 | Turbid | Yes | A | 11.8 |
| 27.4 | 0.3 | IPA | 7.00 | Clear | Yes | A | 16.8 |
| 26.6 | 0.3 | EtOH | 2.00 | Turbid | Yes | G | 16.5 |

### Example 16

### Binary Solvent Crystallizations Using Water as a Co-Solvent

In efforts to evaluate the propensity of Compound 1 for hydrate formation, six water miscible solvents (DMF, NMP, DMA, formamide, AcOH, and MeOH) were chosen for binary solvent crystallizations experiments using water as a co-solvent. Each solvent was pre-mixed with 2% and 10% water, for a total of 12 solvent combinations.

Compound 1 (25-30 mg) was weighed out into vials and the corresponding solvent mixture was added until the material went into solution at elevated temperature (55° C.). After a hot filtration through a syringe filter (Millex-FH), the vials were then placed in a refrigerator and held at 4° C. for 16 hours (fast cooling protocol) or slowly cooled to room temperature at a rate of 20° C./h and further stirred at room temperature for 16 hours (slow cooling protocol). Tables 27 and 28 summarize the experimental details for both sets (fast and slow cooling). The isolated solids were collected by vacuum filtration. The vials without precipitates were evaporated to dryness using a gentle stream of nitrogen. All resultant solids were dried in vacuo at room temperature and 30 inches Hg.

The collected solids were analyzed by XRPD. Both fast and slow cooling experiments showed that aqueous DMF and acetic acid afforded Form B, aqueous formamide afforded Form E, aqueous methanol afforded Form G and aqueous DMA afforded Form D with only one exception of DMA/10% water, which afforded mostly Form B with some extra diffraction peaks. The residual material from aqueous NMP after evaporation under nitrogen flow was not analyzable as the material was an oil.

TABLE 27
Crystallizations with 2% and 10% Water using Fast Cooling Procedure

| Material | | Solvent | | | | |
|---|---|---|---|---|---|---|
| Amount | Solvent/% | Amount | Temp | | | Recovery |
| (mg) | Water | (mL) | (° C.) | Precipitate | Form | (mg) |
| 28.6 | DMF/2% | 0.3 | 55 | No | B | n/a |
| 24.9 | DMF/10% | 0.3 | 75 | Small | B | n/a |
| 30.3 | NMP/2% | 0.3 | 55 | No | n/a | n/a |
| 29.1 | NMP/10% | 0.3 | 55 | No | n/a | n/a |
| 24.5 | DMA/2% | 0.3 | 55 | Yes | D | 10.9 |
| 29.8 | DMA/10% | 0.3 | 55 | Yes | B + D | 12.6 |
| 25.3 | FA/2% | 0.3 | 55 | Yes | E | 11.6 |
| 24.8 | FA/10% | 0.3 | 75 | Yes | E | 10.7 |
| 27.5 | AcOH/2% | 0.3 | 55 | evap to dr. | B | n/a |
| 24.5 | AcOH/10% | 0.3 | 55 | evap to dr. | B+ | n/a |
| 24.6 | MeOH/2% | 7.3 | 64 | Yes | G | 14.0 |
| 27.6 | MeOH/10% | 7.3 | 55 | Yes | G | 13.6 |

FA: Formamide
n/a: not analyzable
B+: Form B with extra diffraction peaks

TABLE 28
Crystallizations with 2% and 10% Water Using Slow Cooling Procedure

| Material | | Solvent | | | | |
|---|---|---|---|---|---|---|
| Amount | Solvent/% | Amount | Temp | | | Recovery |
| (mg) | Water | (mL) | (° C.) | Precipitate | Form | (mg) |
| 25.6 | DMF/2% | 0.3 | 55 | evap to dr. | B+ | n/a |
| 28.6 | DMF/10% | 0.3 | 75 | Yes | B | 8.1 |
| 25.4 | NMP/2% | 0.3 | 55 | evap to dr. | n/a | n/a |
| 29.0 | NMP/10% | 0.3 | 55 | evap to dr. | n/a | n/a |
| 30.0 | DMA/2% | 0.3 | 55 | Yes | D | 11.4 |
| 28.0 | DMA/10% | 0.3 | 55 | Yes | D | 12 |
| 26.5 | FA/2% | 0.3 | 55 | Yes | E | 13.9 |
| 29.5 | FA/10% | 0.3 | 75 | Yes | E | 14.8 |
| 27.4 | AcOH/2% | 0.3 | 55 | Yes | B | 13.2 |
| 29.2 | AcOH/10% | 0.3 | 55 | Yes | B | 12.2 |
| 25.7 | MeOH/2% | 7.3 | 64 | Yes | G | 17.3 |
| 26.3 | MeOH/10% | 7.3 | 55 | Yes | G | 15.2 |

FA: Formamide
n/a: not analyzable

### Example 17

### Reslurry of Form A in 20 Solvents

The reslurry of Compound 1 Form A was conducted in 20 solvents: DMF, NMP, DMA, formamide, acetic acid, MeOH, EtOH, THF, EtOAc, MeCN, acetone, MEK, IPA, water, dioxane, MTBE, IPAc, heptane, CH₂O₂, and toluene. About 50-75 mg of Compound 1 was weighed into 2-dram amber vials. Various amounts of solvents were added to each vial to form slurries which were allowed to stir at room temperature for two weeks. The slurries were then filtered with the help of a gentle nitrogen flow. The samples were further dried in vacuo at room temperature for two hours, except the sample from formamide which was dried in vacuo for about 20 hours.

After two weeks, the samples were filtered and then analyzed by XRPD, DSC, and TGA. The results are summarized in Table 29. Three new forms were found from the slurry studies. These Forms I, K, and L were generated from AcOH, NMP, and DMF, respectively. The slurry samples from other solvents afforded results consistent with the single solvent recrystallizations. Form A remained unchanged after reslurry in THF, EtOAc, MeCN, acetone, MEK, IPA, dioxane, MTBE, IPAc, heptane, CH₂Cl₂, and toluene, most likely due to the poor solubility of SNS-314 mesylate in these solvents. Forms D, E, G, and H were generated from reslurry experiments in DMA, formamide, methanol, and ethanol, respectively. These results are consistent with those obtained in single solvent recrystallizations experiments using these solvents.

TABLE 29
Summary of XRPD, DSC and TGA Data for Slurries of
Compound 1 Form A

| | Form by | | TGA Loss |
|---|---|---|---|
| Solvent | XRPD | DSC Peaks (° C.) | (Wt %) |
| DMF | L | 140, 145(x), 226 | 5.7 |
| NMP | K | 90, 113 | 9.9, 6.0 |
| DMA | D | 91, 173 | 0.5, 0.7 |
| Formamide | E | 156, 210 | 3.9 |
| AcOH | I | 81, 105, 159, 174, 178(x), 219 | 3.1, 3.8, 4.4 |
| MeOH | G | 99, 169, 175(x), 216 | 1.5, 3.0 |
| THF | A | 226 | 0.0 |
| EtOAc | A | 228 | 0.0 |
| MeCN | A | 229 | 0.0 |
| Acetone | A | 228 | 0.0 |
| MEK | A | 229 | 0.0 |
| IPA | A | 229 | 0.0 |
| EtOH | H | 121, 161, 176, 219, 227 | 3.8, 2.4, 1.5 |
| Water | B | 87, 171 | n/a |
| Dioxane | A | 229 | 0.0 |
| MTBE | A | 229 | 0.0 |
| IPAc | A | 228 | 0.0 |
| Heptane | A | 228 | 0.0 |
| DCM | A | 229 | 0.0 |
| Toluene | A | 229 | 0.0 |

n/a: weight loss not available from the TGA thermogram most likely due to small sample amount.

### Example 18

### Humidity-Controlled Form Conversion of Form A and Form B

In an attempt to determine the stability of Compound 1 Forms A and B at different humidity levels, chambers with five different humidities (0, 20, 52, 75, and 95% RH, Table 30) were set up for humidity-controlled form conversion for Form A and Form B samples of Compound 1. These chambers were allowed to equilibrate for at least 24 hours before the Form A and Form B samples of Compound 1 were placed in the chambers. The samples were monitored each week for a total duration of five weeks. Each sample was tested by XRPD, DSC, TGA, and Karl Fisher analysis.

TABLE 30
Set Up for Humidity Chambers

| Humidity (% RH) | Reagent |
|---|---|
| 0 | Drierite |
| 20 | Saturated potassium acetate solution |
| 52 | Saturated NaHSO₄•H₂O solution |
| 75 | Saturated NaCl solution |
| 95 | Saturated Na₂HPO₄•12H₂O solution |

The results obtained during the five weeks are summarized in Tables 31-35. For the first week samples, the XRPD patterns did not change for either Form A or Form B. However, the DSC, TGA, and KF results (Table 31) of Form A in 95% RH seemed to suggest the presence of the hydrate (Form B) in the sample. After the second week, the XRPD pattern of the Form A sample in 95% RH was consistent with Form B instead of Form A. This result, along with the DSC, TGA, and KF results (Table 32) for this sample suggested that Form A converted to Form B at 95% RH in two weeks. No form conversion was observed for Form A samples in the lower humidity (i.e., 0, 25, 52, and 75% RH) chambers or any Form B samples for the duration of five weeks.

TABLE 31
Summary of Analytical Results for Humidity Controlled Form Conversion
for Form A and Form B of Compound 1 - Starting Point and Week 1

| | | | | DSC | TGA | KF |
|---|---|---|---|---|---|---|
| Starting | Humidity | | XRPD | Peaks | Loss | (Wt % |
| Form | (% RH) | Week | Form | (° C.) | (Wt %) | H₂O) |
| A | n/a | 0 | A | 229 | 0.0 | 0.07 |
| A | 0 | 1 | A | 229 | 0.0 | 0.17 |
| A | 20 | 1 | A | 229 | 0.0 | 0.16 |
| A | 52 | 1 | A | 229 | 0.0 | 0.29 |
| A | 75 | 1 | A | 229 | 0.0 | 0.12 |
| A | 95 | 1 | A | 171, 228 | 0.1 | 1.5 |
| B | n/a | 0 | B | 170 | 2.5, 0.2 | 3.0 |
| B | 0 | 1 | B | 120, 170 | 2.4, 0.2 | 1.4 |
| B | 20 | 1 | B | 141, 171 | 2.7, 0.2 | 3.3 |
| B | 52 | 1 | B | 140, 171 | 2.7, 0.2 | 3.0 |
| B | 75 | 1 | B | 146, 172 | 2.3, 0.2 | 3.4 |
| B | 95 | 1 | B | 144, 171 | 2.4, 0.2 | 3.2 |

TABLE 32
Summary of Analytical Results for Humidity Controlled Form Conversion
for Form A and Form B of Compound 1 - Week 2

| | | | | DSC | TGA | KF |
|---|---|---|---|---|---|---|
| Starting | Humidity | | XRPD | Peaks | Loss | (Wt % |
| Form | (% RH) | Week | Form | (° C.) | (Wt %) | H₂O) |
| A | 0 | 2 | A | 229 | 0.0 | <0.1 |
| A | 20 | 2 | A | 229 | 0.0 | 0.12 |
| A | 52 | 2 | A | 229 | 0.0 | 0.16 |
| A | 75 | 2 | A | 230 | 0.0 | 0.13 |
| A | 95 | 2 | B | 126, | 1.7, 0.2 | 3.2 |
| | | | | 171, 226 | | |
| B | 0 | 2 | B | 142, 171 | 3.0, 0.2 | 3.4 |
| B | 20 | 2 | B | 119, 170 | 2.7, 0.3 | 3.7 |
| B | 52 | 2 | B | 130, 170 | 2.7, 0.2 | 3.6 |
| B | 75 | 2 | B | 133, 170 | 2.4, 0.3 | 3.4 |
| B | 95 | 2 | B | 136, 170 | 2.8, 0.3 | 3.5 |

TABLE 33
Summary of Analytical Results for Humidity Controlled Form Conversion
for Form A and Form B of Compound 1 - Week 3

| | | | | DSC | TGA | KF |
|---|---|---|---|---|---|---|
| Starting | Humidity | | XRPD | Peaks | Loss | (Wt % |
| Form | (% RH) | Week | Form | (° C.) | (Wt %) | H₂O) |
| A | 0 | 3 | A | 229 | 0.0 | 0.11 |
| A | 20 | 3 | A | 229 | 0.0 | 0.11 |
| A | 52 | 3 | A | 229 | 0.0 | 0.11 |
| A | 75 | 3 | A | 229 | 0.0 | 0.19 |
| A | 95 | 3 | B | 130, 171 | 2.1, 0.1 | 3.7 |
| B | 0 | 3 | B | 142, 171 | 2.5, 0.4 | 3.6 |
| B | 20 | 3 | B | 119, 170 | 2.9, 0.3 | 3.6 |
| B | 52 | 3 | B | 130, 170 | 2.9, 0.3 | 3.7 |
| B | 75 | 3 | B | 133, 170 | 2.8, 0.3 | 2.4 |
| B | 95 | 3 | B | 136, 170 | 3.2 | 4.6 |

TABLE 34
Summary of Analytical Results for Humidity Controlled Form Conversion
for Form A and Form B of Compound 1 - Week 4

| | | | | DSC | TGA | KF |
|---|---|---|---|---|---|---|
| Starting | Humidity | | XRPD | Peaks | Loss | (Wt % |
| Form | (% RH) | Week | Form | (° C.) | (Wt %) | H₂O) |
| A | 0 | 4 | A | 229 | 0.0 | <0.1 |
| A | 20 | 4 | A | 228 | 0.0 | <0.1 |
| A | 52 | 4 | n/a | n/a | n/a | n/a |
| A | 75 | 4 | A | 229 | 0.0 | <0.1 |
| A | 95 | 4 | B | 122, 171 | 1.5 | 3.5 |
| B | 0 | 4 | B | 140, 171 | 2.8, 0.2 | 3.0 |
| B | 20 | 4 | B | 137, 170 | 2.7, 0.3 | 3.4 |
| B | 52 | 4 | B | 140, 171 | 2.5, 0.3 | 3.3 |
| B | 75 | 4 | B | 132, 171 | 2.6, 0.3 | 3.3 |
| B | 95 | 4 | B | 131, 170 | 2.3, 0.3 | 3.7 |

n/a: sample not available.

TABLE 35
Summary of Analytical Results for Humidity Controlled Form Conversion
for Form A and Form B of Compound 1 - Week 5

| | | | | DSC | TGA | KF |
|---|---|---|---|---|---|---|
| Starting | Humidity | | XRPD | Peaks | Loss | (Wt % |
| Form | (% RH) | Week | Form | (° C.) | (Wt %) | H₂O) |
| A | 0 | 5 | A | 229 | 0.0 | 0.1 |
| A | 20 | 5 | A | 229 | 0.0 | 0.2 |
| A | 52 | 5 | n/a | n/a | n/a | n/a |
| A | 75 | 5 | A | 229 | 0.0 | 0.3 |
| A | 95 | 5 | B | 126, 172 | 2.3, 0.2 | 3.9 |
| B | 0 | 5 | B | 143, 172 | 3.0, 0.3 | 3.4 |
| B | 20 | 5 | B | 128, 170 | 2.4, 0.3 | 3.5 |
| B | 52 | 5 | B | 120, 170 | 2.2, 0.3 | 3.6 |
| B | 75 | 5 | n/a | n/a | n/a | n/a |
| B | 95 | 5 | B | 148, 172 | 2.6, 0.3 | 4.7 |

n/a: sample not available.

### Example 19

### Ripening Experiments and Relative Stability of Forms

In order to further investigate the relative stability of Forms A, B, E, and G, ripening experiments were performed in water and MEK as detailed in Table 36. In these experiments 10 to 40 mg of the samples were weighed into amber vials, and 0.8 mL water or 1 mL MEK was dispensed into each vial to form slurries. MEK was briefly dried using dried molecular sieves. The KF results showed 0.4 wt % of water in MEK after drying. The vials were capped with Teflon lined caps and sealed using a Parafilm® tape. After a week of stirring, the slurries were sampled, filtered, and analyzed by XRPD.

TABLE 36
Ripening Studies of Forms A, B, E, and G of Compound 1

| | Sample Amount | | |
|---|---|---|---|
| Solvent | (mg) | Starting Form | Final Form |
| Water | 20 | B | B |
| Water | 20 | E | B |
| Water | 20 | G | B |
| Water | 10 + 10 | A + B | B |
| Water | 10 + 10 | A + E | B |
| Water | 10 + 10 | A + G | B |
| Water | 10 + 10 | B + E | B |
| Water | 10 + 10 | B + G | B |
| Water | 10 + 10 | E + G | B |
| MEK | 20 | B | B |
| MEK | 20 | E | B |
| MEK | 20 | G | A |
| MEK | 10 + 10 | A + B | B |
| MEK | 10 + 10 | A + E | B |
| MEK | 10 + 10 | A + G | A |
| MEK | 10 + 10 | B + E | B |
| MEK | 10 + 10 | B + G | B |
| MEK | 10 + 10 | E + G | A |

The XRPD analysis showed that all slurries in water generated Form B. These results were consistent with the observations made during the polymorph study and during the process development studies.

The slurries in MEK starting with Form G, Forms A+G, or Forms E+G converted to Form A. The other six slurries in MEK (B, E, A+B, A+E, B+E, B+G) all converted to Form B. These results suggested that Form G is less stable than Form A and Form B. The slurry started with Forms A+B converted to Form B, indicating Form B is more stable than Form A.

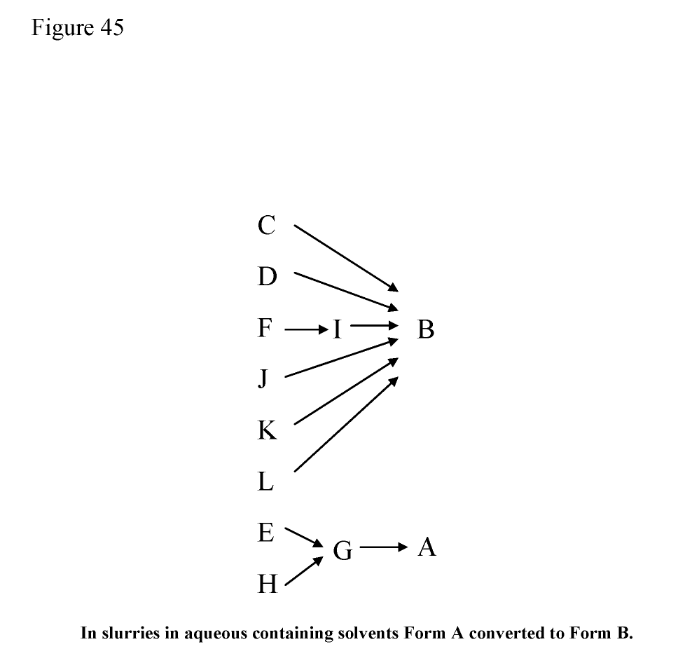

Based on the characterization of various forms of Compound 1, the relative stability of the forms (A to L) can be ranked as shown in FIG. 45. Detailed description and conversion conditions between the forms are summarized in Table 37.

TABLE 37
Description and Relative Stability of Crystalline Forms of Compound 1

| Form | Description | Converts to | Conditions or Comments |
|---|---|---|---|
| A | Anhydrate | B | In water containing solvents or in anhydrous |
| | | | solvents with Form B seeding |
| B | Monohydrate | A | In anhydrous acetone (with distillation) |
| C | n/a | B | Under normal storage conditions |
| D | DMA solvate | B | At above 60% RH |
| E | Formamide | G | After drying at 105° C. in vacuo |
| | solvate | | |
| F | n/a | I | Crystallizations or slurries in AcOH all |
| | | | generated Form I, except one experiment |
| | | | which generated Form F, indicating Form F |
| | | | is less stable than Form I |
| G | Monohydrate | A (or B) | Converts to From A in anhydrous solvents; |
| | | | converts to From B in water containing |
| | | | solvents or anhydrous solvent with From B |
| | | | seeding |
| H | Ethanol solvate | G | At above 90% RH |
| I | AcOH solvate or | B | At above 50% RH |
| | hydrate | | |
| J | DMF solvate or | B | At above 40% RH |
| | hydrate | | |
| K | NMP solvate | B | Gradually converts to Form B under humid |
| | | | environment. |
| L | DMF solvate | B | At above 80% RH |

n/a: identification not available due to lack of materials.

### Example 20

### Kinase Assays

Compound 1 was tested for inhibitory activity against a panel of 219 kinases (Upstate Biotechnology, Dundee, UK). All screens were performed by incubating the kinase enzyme, Compound 1, and radiolabeled ATP together for typically 30-60 min. The final ATP concentration in the reaction was within 15 mM of the Kₘ for ATP, as calculated by Upstate.

It was determined that Compound 1 is a highly selective Aurora kinase inhibitor. Only 7 kinases out of the 219 show selectivity less than 100-fold. The respective IC₅₀ values for these kinases are shown in Table 38.

TABLE 38

| Kinase | IC₅₀ (μM) in Radiometric Assay |
|---|---|
| Aurora A | 0.001 |
| TrkB | 0.005 |
| TrkA | 0.012 |
| Flt4 | 0.014 |
| Fms | 0.015 |
| DDR2 | 0.082 |
| Axl | 0.084 |
| c-Raf | 0.100 |

Fourteen other kinases had an IC₅₀ value between 0.100 μM and 1 μM. Compound 1 showed at least a 1000-fold selectivity over the remaining 197 kinases (i.e., IC₅₀≧1 μM). These data suggest that Compound 1 has a low potential for off-target kinase related toxicities.

### Example 21

### Aurora Biochemical Assays

A Homogenous Time-Resolved Fluorescence (HTRF)-based biochemical IC₅₀ assay from Cisbio (Bedford, Mass.) was used to test for the kinase activity of the three isoforms of Aurora (Aurora A, B, and C) in the presence of Compound 1. A biotin-conjugated histone H3 peptide (Upstate Biotechnology) was used as a substrate.

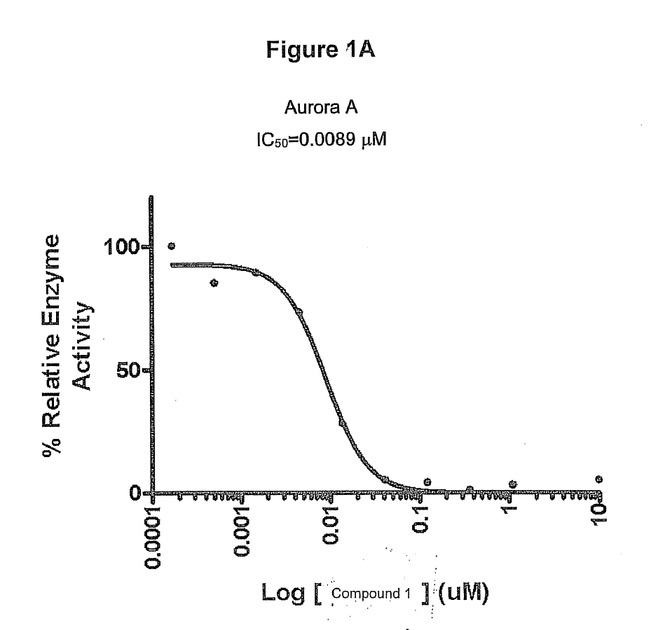

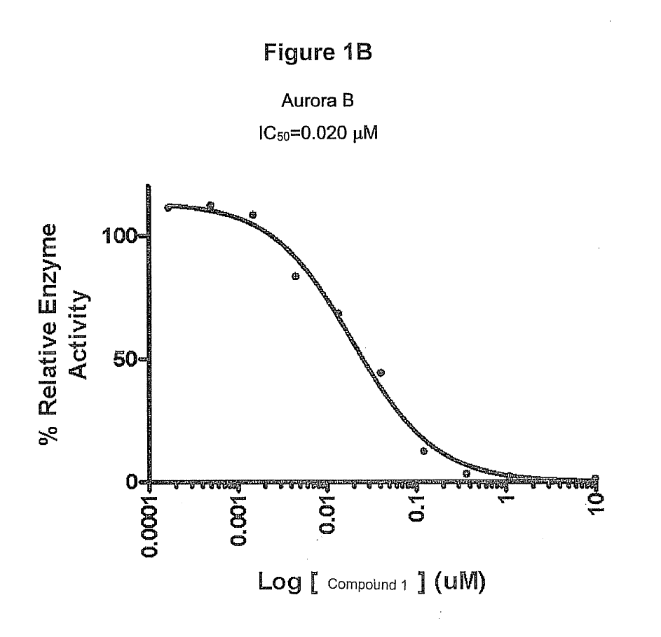

FIG. 1 shows representative Compound 1 IC₅₀ curves for (A) Aurora A and (B) Aurora B using the HTRF-based biochemical assay. As can be seen in this Figure, Compound 1 has an IC₅₀ of 0.0089 μM for Aurora A, and has an IC₅₀ of 0.020 μM for Aurora B.

Table 39 shows a summary of the results using the HTRF assay for Aurora A, Aurora B, and Aurora C. It can be seen from the data that Compound 1 is a potent Aurora kinase inhibitor.

TABLE 39

| | Aurora-A | Aurora-B | Aurora-C |
|---|---|---|---|
| Average IC₅₀ (μM) | 0.009 | 0.031 | 0.0034 |
| Standard Deviation (SD) | 0.002 | 0.007 | NA |
| N | 9 | 10 | 1 |

### Example 22

### Crystallography

Diffraction-quality crystals of Aurora A in complex Compound 2 were obtained by hanging-drop vapor diffusion at 20-25° C. Diffraction data were collected under standard cryogenic conditions on RAXIS-IV, processed and scaled by using CrystalClear from Rigaku/Molecular Structure Corporation. The structures were determined from single-wavelength native diffraction experiments by molecular replacement with AMoRe using a search model from a previously determined structure.

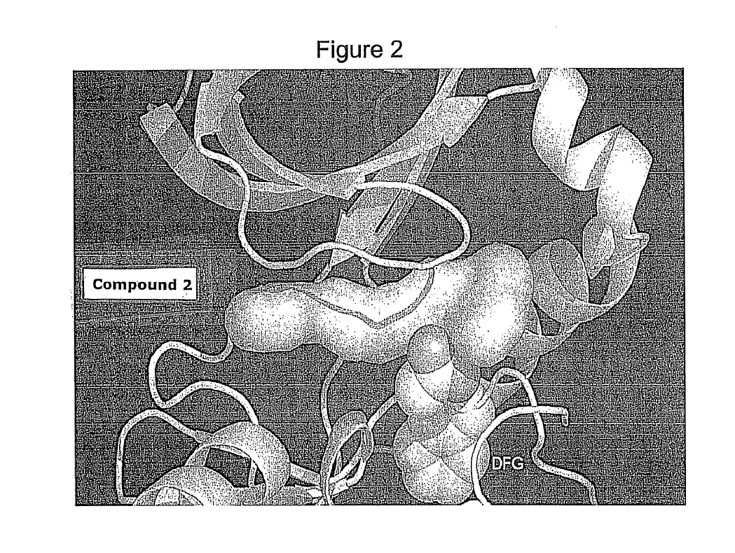

A detail of a crystal structure of Aurora A with Compound 2 is provided in FIG. 2. It can be seen from the structure that the compound is in an extended conformation. In particular, the inhibitor is located in the ATP (purine) binding pocket and extends into the substrate binding groove. Furthermore, the compound binds to the active conformation of Aurora A.

### Example 23

### Flow Cytometry

HCT 116 cells were seeded at 10,000 cells per well in 12-well plates and cells were incubated 24 hr at 37° C. Compound 1 compound titration was achieved by making a 3-fold dilution series [in dimethyl sulfoxide (DMSO)], starting at 10 mM for a total of 11 concentrations (10 mM-0.0002 mM) and one DMSO control. This series was diluted 1000× in RPMI-1640 containing 10% FBS (1× treatment concentration: 10 μM-0.0002 μM).

Plates were removed from the incubator, growth media was aspirated, and 1 mL/well of 1× Compound 1 compound dilution series (in RPMI-1640/10% FBS) or no treatment control (RPMI-1640/10% FBS/0.1% DMSO) was added to cells. After 16 hrs, media was aspirated and placed in a labeled collection tube, cells were trypsinized with 100 μL trypsin for 5 min at room temperature, quenched with fresh media, and placed in the collection tube with their appropriate media aspirate. Cells were spun at 2000 RPM for 5 min, supernatant was aspirated, and cells were re-suspended in 50 μl, 1× phosphate buffered saline (PBS) and 200 μL 100% methanol. Samples were then placed at −20° C. Cells fixed in methanol were spun at 2000 RPM for 5 min, supernatant was removed and cells were washed with 500 μL 0.1% bovine serum albumin (BSA) in PBS. Cells were re-suspended in 100 μL propidium iodide (PI) staining solution [prepared from 10 μg/mL PI (Sigma #P4864), 100 μg/mL RNase (Sigma #R4642) in PBS] and incubated at 37° C. for 1 hr. Cell populations were then analyzed by flow cytometry on Fluorescence-Activated Cell Sorter (FACS) instrumentation (FACSCalibur; Becton-Dickinson) according to common techniques.

The distribution of cells in the various phases of cell cycle was assessed by propidium iodide (PI) staining of DNA. The total intensity of PI was considered to reflect the DNA content of cells.

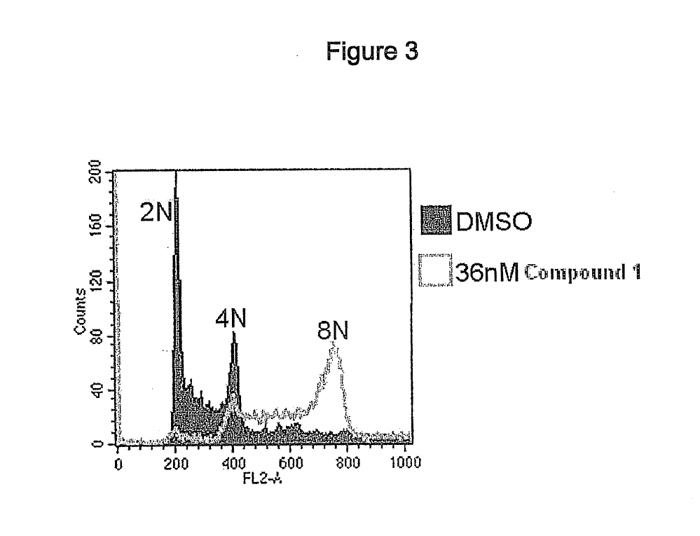

Data is shown in FIG. 3 from the 36 nM treatment sample. As can be seen from the Figure, exposure to Compound 1 caused cells to have a higher DNA content than exposure to DMSO vehicle. While the DMSO control-treated cells were predominantly distributed across 2N (G1) and 4N (G2/M) peaks, cells treated with 36 nM Compound 1 had predominantly 4N and 8N DNA content. This phenomenon is indicative of aberrant mitosis.

### Example 24

### Fluorescent Imaging

HCT 116 cells were seeded at 6×10⁴ cells/mL on coverslips in 12-well plates, and were treated with 16 nM Compound 1 or DMSO control for 72 hr. Cells were then fixed with 4% paraformaldehyde for 20 min at room temperature, washed with 1×PBS three times, permeabilized with 0.1% Triton® X-100 nonionic surfactant for 5 min at room temperature, washed with 1×PBS twice, blocked with 10% fetal bovine serum (FBS) in PBS for 2 hr at room temperature. The cells were incubated in a diluted alpha-tubulin primary antibody solution in 10% FBS for 2 days at 4° C., and stained with DAPI (DNA/.blue) and with a diluted FITC-labeled secondary antibody (tubulin/green) solutions in 10% FBS for 1 hr at room temperature away from light. Cells were then washed in 1×PBS and the coverslips were mounted on slides and analyzed with a Leica DMIRE2 fluorescence microscope with a 63× oil immersion objective. Images were captured on a Leica DFC300FX CCD camera and analyzed using Image-Pro software. For both images captured, the same objective was used.

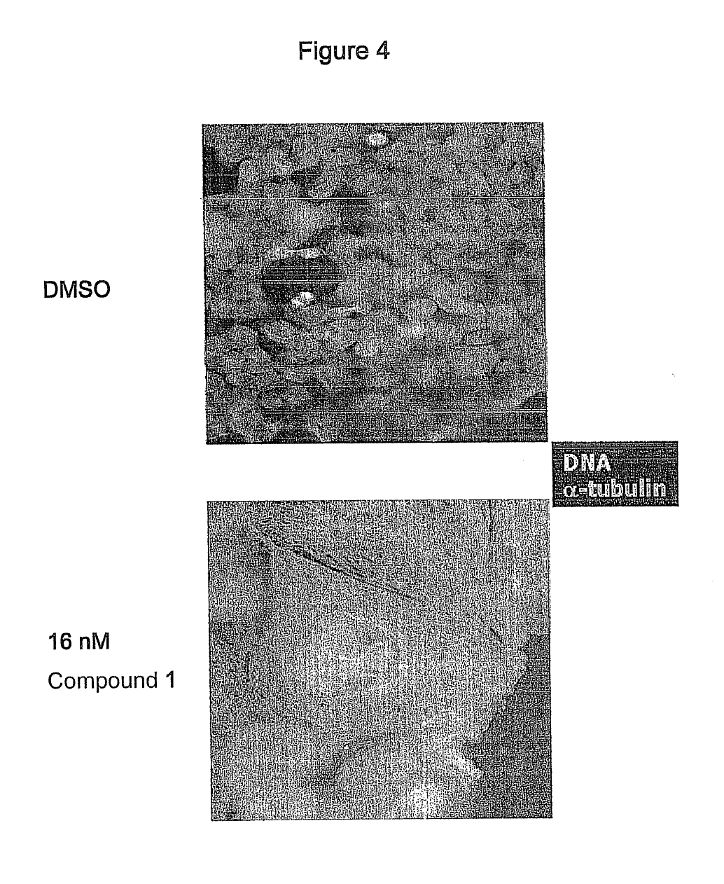

As shown in FIG. 4, treatment of the cells with the compound caused formation of large polyploid cells. A drastic increase in both nuclear and cellular area was observed when cells were treated with 16 nM Compound 1 for 72 hr as compared to vehicle. This increase in nuclear and cellular area indicates that the compound causes cell cycle defects that lead to abnormal cytokinesis and endoreduplication. These defects are consistent with Aurora kinase inhibition.

### Example 25

### Phospho-Histone H3 Staining (High Content Screening)

Analysis of phospho-histone H3 (pHH3) levels was performed on adherent cells using high-content screening methodology. HCT 116 cells were plated at 1,000 cells per well in growth medium on 96-well poly-L-lysine plates and allowed overnight growth at 37° C. Compound 1 titration was achieved by making a 3-fold dilution series (in DMSO) starting at 10 mM for a total of 11 concentrations (10 mM-0.0002 mM) and one DMSO control. This series was diluted 1000× in RPMI-1640 containing 10% FBS (1× treatment concentration: 10 μM-0.0002 μM). Plates were removed from the incubator, growth media was aspirated, and 100 μL/well of 1× compound dilution series (in RPMI-1640 with 10% FBS) or no treatment control (RPMI-1640 with 10% FBS/0.1% DMSO) was added to cells in duplicate wells.

Cells were treated with the various concentrations of Compound 1 for 1 hour. Then, medium was aspirated and cells were incubated in 100 μL/well 4% formaldehyde for 15 min at room temperature. After aspirating the fixation solution, cells were rinsed once in 100 μL/well 1×PBS and then incubated in 100 μL/well permeabilization buffer (0.5% Triton X-100 in 1×PBS for 5 min at room temperature. This solution was aspirated and 100 μL/well of blocking buffer (10% FBS in 1×PBS) was added. Cells were incubated for 10-20 min at 37° C. After aspirating the blocking buffer, the cells were incubated in 50 μL/well primary antibody solution (p-Histone H3 Cell Signaling # 9701 at 1:400 in 10% FBS) for 1-2 hr at 37° C. Antibody solution was removed and cells were washed twice in 100 μL of 1×PBS. After removing the PBS, cells were incubated in 50 μL/well staining solution (1:100 secondary antibody/1:5000 Hoechst stain) for 35 min at room temperature away from light. Finally, cells were washed 3 times with 200 μL/well 1×PBS. Images were captured and pHH3 staining was analyzed using the Target Activation application and ArrayScan VTI™ instrument (Cellomics, Inc.). Data points taken from the parameter Mean_AveIntenCh2 were graphed in GraphPrism and fitted into an IC₅₀ equation.

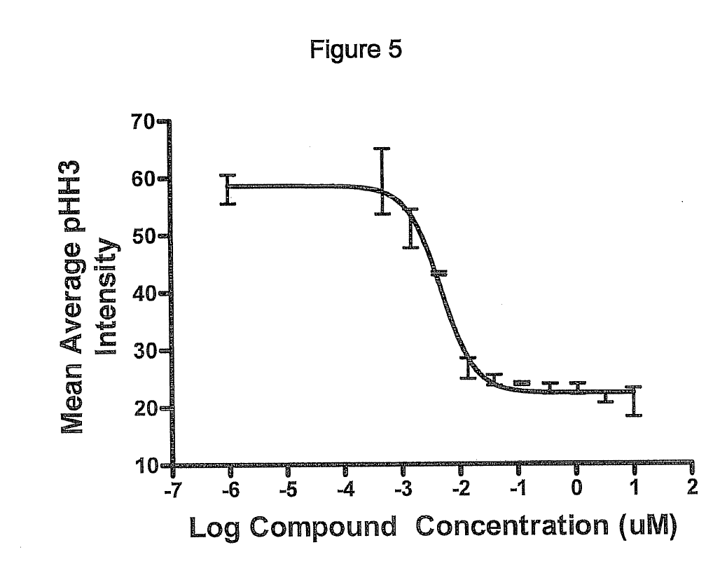

As can be seen in FIG. 5, phosphorylation of histone H3 on serine 10, a known Aurora B cellular target, was inhibited by treatment of cells with Compound 1. The EC₅₀) of the reduction of pHH3 is approximately 9 nM. The reduction in pHH3 levels likely reflects inhibition of Aurora B activity in HCT 116 cells by the compound.

### Example 26

### Cellular Profile

Cellular proliferation was assessed using the Cell Proliferation ELISA, bromodeoxyuridine (BrdU) kit (Roche) including reagents, according to the kit protocol. Briefly, cells were treated with Compound 1 for 96 hr and labeled with BrdU for 2 hr before preparation for analysis.

For cell cycle analysis on adherent cells (HCT116, Calu-6, PC3, HeLa, A375, MiaPaca2, MDA-MB-231, and H1299), tumor cells were grown in 96-well tissue culture plates overnight at 37° C. The cells were then exposed to Compound 1 at 0.0002 to 10 μM for 16 hours. Cells were fixed, stained, and analyzed. The percentage of cells with ≧4N DNA content as a function of concentration was fit to estimate EC₅₀. For cell cycle analysis on nonadherent cells or cells with irregular morphology (A2780, HL-60, CCRF-CEM, and HT-29), tumor cells were seeded in 12-well tissue culture plates overnight at 37° C. The cells were then exposed to Compound 1 at 0.0002 to 10 μM for 16 hours. Cells were trypsinized, collected, stained with propidium iodide, and analyzed by flow cytometry.

For analysis of pHH3 on adherent cells (HCT116, A375, and H1299), tumor cells were grown in 96-well tissue culture plates overnight at 37° C. The cells were then exposed to Compound 1 at 0.0002 to 10 μM for 1 hour. Cells were fixed, permeabilized and exposed to anti-pHH3 antibody and analyzed for pHH3 staining. Data were fit to an IC₅₀ equation. For analysis of pHH3 on nonadherent cells or cells with irregular morphology (A2780, Calu-6, and HT-29), tumor cells were seeded in 6-well tissue culture plates overnight at 37° C. The cells were then exposed to Compound 1 at 0.0002 to 10 μM for 1 hour. Cells were trypsinized, collected, lysed, and analyzed by immunoblotting.

For mitotic indexing, solid tumor cells were grown in 96-well tissue culture plates overnight at 37° C. Cells were fixed, permeabilized, and exposed to fluorescently labeled antibody MPM2. The percentage of cells staining positive with this antibody was analyzed.

For analysis of Aurora A and Aurora B levels, tumor cells were grown in 12-well tissue culture plates overnight at 37° C. Cells were harvested, separated by SDS-PAGE electrophoresis, and total Aurora kinase levels were analyzed by immunoblotting with antibodies to Aurora A and Aurora B.

The cellular effects of Compound 1 in a diverse panel of tumor cell lines are provided in Table 40.

TABLE 40

| | | | Cell | | | |
|---|---|---|---|---|---|---|
| | | BrdU | Cycle | pHH3 | MPM2⁺ | Aurora A |
| Tumor | | IC₅₀ | EC₅₀ | IC₅₀ | Cells | vs. B |
| Type | Cell Line | (μM) | (μM) | (μM) | (%) | Score^{a} |
| Colon | HCT 116 | 0.0064 | 0.034 | 0.009 | 4.149 | 3 |
| | HT29 | 0.0244 | 0.012 | 0.06 | 2.95 | 1 |
| Lung | Calu-6 | 0.0133 | 0.013 | 0.06 | 1.928 | 2 |
| | H1299 | 0.004 | 0.08 | 0.058 | 2.955 | 1 |
| Prostate | PC3 | 0.0044 | 0.011 | — | 0.361 | 1 |
| Ovarian | A2780 | 0.0018 | 0.0061 | 0.15 | 3.413 | 3 |
| Breast | MDA- | 0.0081 | 0.03 | — | 0.4275 | 1 |
| | MB-231 | | | | | |
| Cervical | HeLa | 0.0093 | 0.29 | — | 2.25 | 1 |
| Pancreatic | MiaPaca2 | 0.0091 | 0.009 | — | 1.614 | — |
| Melanoma | A375 | 0.0059 | 0.093 | 0.015 | 1.115 | 2 |
| Leukemia | HL-60 | —^{b} | 0.007 | — | — | — |
| | CCRF- | — | 0.0042 | — | — | 2 |
| | CEM | | | | | |

^{a}Score of 1: Aurora A levels > Aurora B levels 2: Aurora A levels = Aurora B levels 3: Aurora A levels < Aurora B levels
^{b}A dash (—) indicates not tested.

It can be seen from Table 15 that Compound 1 shows low nanomolar anti-proliferative activity in a broad panel of cancer cell lines, with IC₅₀ values between 0.002 μM and 0.01 μM. Compound 1 also potently inhibits normal progression of cell cycle, and the phosphorylation of histone H3. The potency of Compound 1 in the assays of this example is independent of Aurora A and Aurora B levels, and the mitotic indicies.

### Example 27

### In Vivo Mouse Xenograft Assays

The studies in Examples 20, 21, and 22 used female mice nu/nu athymic mice. Compound 1 was formulated fresh each week for dosing. The powder containing Compound 1 was added directly to a 30% aqueous cyclodextrin solution and sonicated at 50° C. for approximately 30 min until dissolved.

HCT 116 colorectal carcinoma cells were implanted in the animals' right hind flanks subcutaneously with 200 μL of a 2.5×10⁷ cells/mL suspension [1:1 Dulbecco's PBS (DPBS) with cells:Matrigel™. For each of the studies of compound distribution, pHH3 levels, and tumor section microscopy, after the tumors reached an average volume of 500 mm³, the animals were weighed and sorted into randomized groups before initial dosing. Dosing schedules are provided separately for each of the studies in Examples 28, 29, and 30.

### Example 28

### Distribution of Compound In Vivo

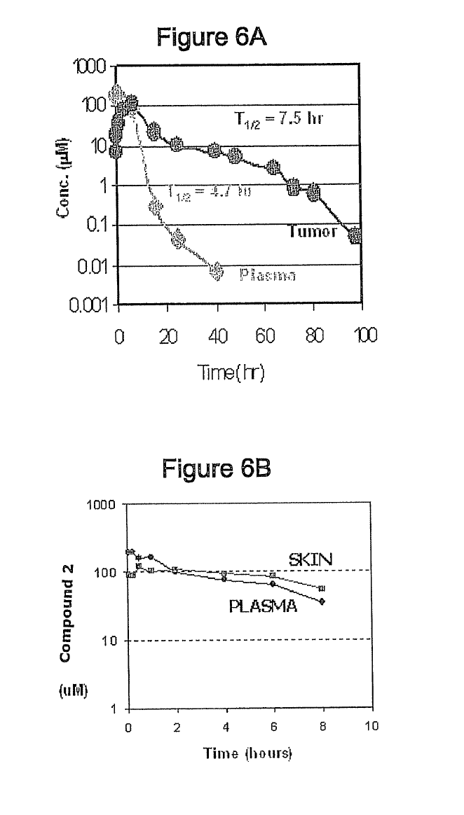

For the distribution studies depicted in FIG. 6A, the mice were treated with a single dose of 170 mg/kg of Compound 1 intraperitoneally (IP). Terminal blood and tumor samples were harvested between 15 min and 96 hr.

Female nu/nu athymic mice received HCT 116 colorectal cancer cell suspension (1:1 DPBS with cells:Matrigel) as a subcutaneous injection in the right hind flank. When tumors reached an average volume of 500 mm³, mice were sorted into groups of 3 per time point. Compound 2 was extracted from tumor after homogenization with 10×w/v PBS. Quantification of Compound 2 was done by HPLC-MS/MS after extraction from plasma and tumor homogenate with acetonitrile. For HPLC-MS/MS, the detector consisted of an API4000 (Sciex/ABI, Foster City, Calif.) triple quadrapole mass spectrometer using turbo electrospray ionization. Half-life estimates were made using the last 5 time points in tumor and last 3 time points in plasma.

It can be seen from FIG. 6A that Compound 2 was preferentially retained in the tumor, i.e., the half-life of the compound is longer in the tumor as compared with its half-life in plasma (7.5 hr versus 4.7 hr, respectively).

For the distribution studies shown in FIG. 6B, Female nu/nu athymic mice were administered 170 mg/kg Compound 1 IP with terminal plasma and skin collections between 15 min-16 hr post administration. It can be seen from FIG. 6B that the plasma and skin PK profiles are similar. The similar profile allows PD readouts in the skin to be directly correlated with drug concentrations measured in the plasma.

### Example 29

### Phospho-Histone H3 In Vivo

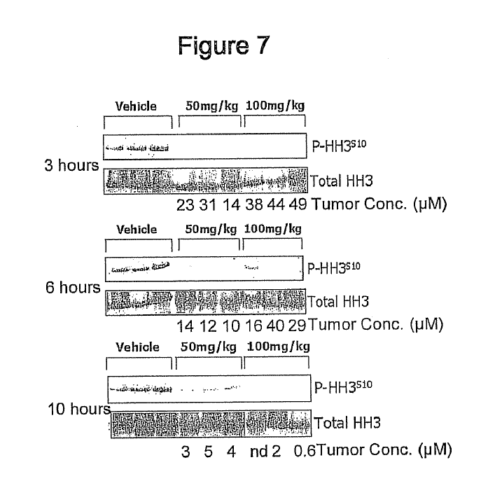

For the pHH3 studies depicted in FIG. 7, the mice were treated IP with a single dose of either vehicle, 50 mg/kg of Compound 1, or 100 mg/kg of Compound 1, as labeled. It can be seen that at the 50 mg/kg and 100 mg/kg doses of Compound 1, the level of pHH3 is decreased at 3 hr, 6 hr, and 10 hr post administration, as compared with the levels observed in vehicle-treated mice. The levels of compound in the tumor are provided below each lane; the levels of compound in the tumor are more than 20 times greater than the IC₅₀ for Aurora B in vitro.

### Example 30

### Microscopy of Tumor Sections

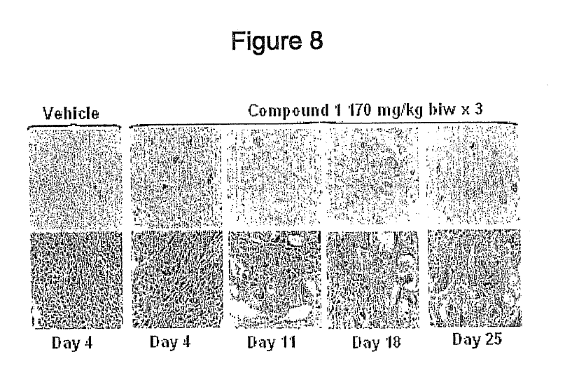

For the microscopy assays depicted in FIG. 8, the mice were treated either with vehicle or with a dose of Compound 1 of 170 mg/kg twice-weekly for three weeks. Following the treatment, tumors were harvested, placed in Streck fixative, paraffin embedded, sectioned, and transferred to slides. Tumor sections were stained with hematoxylin and eosin (H&E). Hematoxylin stains negatively charged nucleic acid structures, such as nuclei and ribosomes, blue, whereas eosin stains proteins pink. Treatments were administered on Day 1, 4, 8, 11, 15, and 18, with tumors being excised Day 4, 11, 18, and 25 of the study. All images in this Figure were taken at 40× magnification.

As the upper panel of FIG. 8 demonstrates, a significant increase (compared to vehicle) of caspase-3 positive cells were observed up to 18 days, indicating induction of apoptosis. As the lower panel of FIG. 8 demonstrates, large polyploid cells appeared by Day 4 and persisted for at least 25 days after treatment initiation, indicating successive rounds of endoreduplication.

### Example 31

### In Vivo Efficacy

HCT 116 colon cancer cells [200 μL of a 2.5×10⁷ cells/mL suspension (1:1 DPBS with cells:Matrigel)] were subcutaneously implanted in the right hind flank of female nu/nu athymic mice. After 7 days, when tumors reached an average volume of approximately 200 mm³, animals were weighed, randomized by tumor volume (l×w×h×0.52), and assigned to the various study groups before initial dosing.

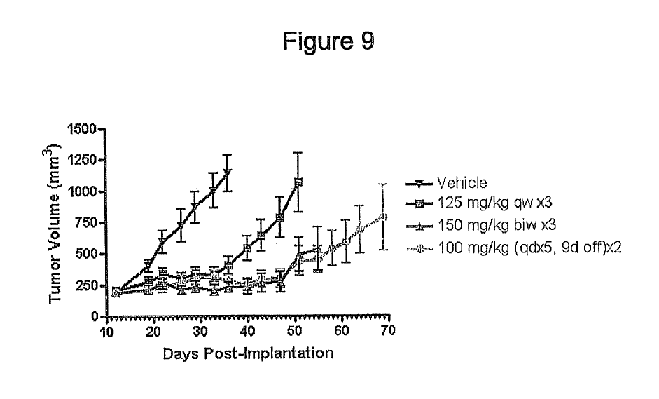

Compound 1 was tested for efficacy in HCT 116 xenograft mice on the following three schedules: a twice-weekly (biw) schedule for three weeks, a once-weekly (qw) schedule for three weeks, and a schedule of daily treatment for five days with a 9-day interval without drug administration (qd x5, 9 day off) with two cycles administered. The animals on the twice-weekly schedule received compound on Days 1, 4, 8, 11, 15 and 18. Doses were as shown in FIG. 9 and in Table 41. It can be seen from this Figure and the table that Compound 1 shows strong anti-tumor activity in HCT 116 xenograft mice on all dosing schedules tested.

TABLE 41

| Dose | | % TGI | TGD |
|---|---|---|---|
| (mg/kg) | Schedule | (Day 36) | (days) |
| 125 | Qw × 3 | 79.8 | 22.5 |
| 150 | biw × 3 | 95.6 | 32.5 |
| 100 | qd × 5, | 91.6 | 45 |
| | 9 d off × 2 | | |

TGI = Tumor Growth Inhibition
TGD = Tumor Growth Delay

Tumor Growth Inhibition (TGI) was determined by examining the tumor volume graph and calculating the percent of inhibition from the vehicle control group on the last day the control contained at least 75% of the animals. Percent TGI is then calculated with the following equation:

$$\% TGI = \frac{\begin{matrix}( control {TV}_{t} - control {TV}_{i} ) - \\ ( treatment {TV}_{t} - treatment {TV}_{i} )\end{matrix}}{( control {TV}_{t} - control {TV}_{i} )} \times 100$$

where TVₜ is the average tumor volume on Day 10 and TV, is the initial average tumor volume. ANOVA was performed to calculate statistical significance, defined as p<0.05.

Time To Endpoint (TTE) was calculated for each individual animal to reach the predetermined study end point where the tumor volume becomes 1200 mm³ or 10% of body weight or a greater than 20% body weight loss for two sequential measurements. The TTE is calculated and the median value is recorded for the group. Tumor Growth Delay (TGD) is then calculated with the following equation:

TGD=median TTEₜᵣₑₐₜₘₑₙₜ−median TTE_{control}

Percent Tumor Growth Delay (% TGD) is calculated with the following equation:

$$\% TGD = \frac{median {TTE}_{treatment} - median {TTE}_{control}}{median {TTE}_{control}} \times 100$$

A Log Rank test was performed to calculate statistical significance, defined as p<0.05.

The three dosing schedules for Compound 1 described above for the HCT 116 xenograft mice were also examined in mouse xenograft assays using other tumor types. Results for the twice-weekly for three weeks schedule and doses used are provided in Table 5 below.

A2780 ovarian cancer cells [200 μL of a 2.5×10⁷ cells/mL suspension (1:1 DPBS with cells:Matrigel)] were implanted subcutaneously in the right hind flank of mice. After 7 days, when tumors reached an average volume of approximately 130 mm³, animals were weighed, randomized by tumor volume (l×w×h×0.52), and assigned to the various study groups before initial dosing.

A375 melanoma tumor fragments (1 mm³) were implanted subcutaneously in the right hind flank of mice. After 9 days, when tumors reached an average volume of approximately 110 mm³, animals were weighed, randomized by tumor volume (l×w×h×0.52), and assigned to the various study groups before initial dosing.

MDA-MB-231 breast cancer cells [200 μL of a 2.5×10⁷ cells/mL suspension (1:1 DPBS with cells:Matrigel)] were implanted subcutaneously in the right hind flank of mice. After 13 days, when tumors reached n average volume of approximately 95 mm³, animals were weighed, randomized by tumor volume (l×w×h×0.52), and assigned to the various study groups before initial dosing.

H1299 non-small cell lung cancer cells [200 μL of a 5×10⁷ cells/mL suspension (1:1 DPBS with cells:Matrigel)] were implanted subcutaneously in the right hind flank. After 10 days, when tumors reached an average volume of approximately 100 mm³, animals were weighed, randomized by tumor volume (l×w×h×0.52), and assigned to the various study groups before initial dosing.

Calu 6 lung carcinoma cells [200 μL of a 5×10⁷ cells/ml suspension (1:1 DPBS with cells:Matrigel)] were implanted subcutaneously in the right hind flank of mice. After 11 days, when tumors reached an average volume of approximately 150 mm³, animals were weighed, randomized by tumor volume (l×w×h×0.52), and assigned to the various study groups before initial dosing.

PC3 prostate tumor fragments (1 mm³) were implanted subcutaneously in the right hind flank of mice. After 21 days, when tumors reached a volume of approximately 120 mm³, animals were weighed, randomized by tumor volume (l×w×h×0.52), and assigned to the various study groups before initial dosing.

As shown in Table 42, Compound 1 effected significant tumor growth inhibition in a dose-dependent manner ranging from 58-99% at well tolerated doses in a variety of mice xenograft models representing a range of tissue types.

TABLE 42

| | Dose | | TGD |
|---|---|---|---|
| Cell line | (mg/kg@ biw × 3) | % TGI | (days) |
| HCT 116 | 150 | 95.6 | 32.5 |
| (Colon) | 100 | 79.4 | 25.1 |
| | 75 | 67.5 | 22 |
| A2780 | 170 | 53.8 | 9 |
| (Ovarian) | 85 | 9.8 | 3.5 |
| | 42.5 | 0 | 0 |
| A375 | 170 | 65.4 | 7.4 |
| (Melanoma) | 85 | 20.6 | 0 |
| | 42.5 | 1.2 | 0 |
| MDA-MB-231 | 170 | 73.8 | 14.1 |
| (Breast) | 85 | 19.2 | 4.0 |
| | 42.5 | 20.6 | 3.5 |
| H1299 | 170 | 69.1 | 5.7 |
| (NSCLC) | 85 | 17.7 | 0 |
| | 42.5 | 0 | 0 |
| CALU6 | 170 | 91.4 | ND |
| (NSCLC) | 85 | 28.7 | ND |
| | 42.5 | 23.5 | ND |
| PC3 | 170 | 67.5 | 25 |
| (Prostate) | 85 | 63.6 | 12 |
| | 42.5 | 39.5 | 0 |

### Example 32

The human cell line MV-4-11 (human acute myeloid leukemia) was established as subcutaneous xenografts in nu/nu female mice. Animals were randomized by tumor volume and distributed into groups of ten animals each. Treatments were initiated when tumors averaged about 200 mm³ in volume. End points for each group were determined based on body weight nadir, adverse clinical observations, or tumor volumes exceeding maximum threshold of 2000 mm³.

Compound 1 was administered intraperitoneally (IP) biweekly (i.e. twice-weekly) for 3 weeks at a dose of 150 mg/kg. Responses were assessed by tumor growth inhibition (TGI) and tumor growth delay (TGD). TGI and TGD in the treatment group were evaluated against the vehicle control group. The treatment significantly delayed tumor growth compared to the vehicle. Percent tumor growth inhibition (% TGI) was 75.56 with a p-value of 0.0008, and the tumor growth delay was 10 days.

### Example 33

### Nonclinical Pharmacokinetics, Distribution, and Excretion

Pharmacokinetic studies were conducted in mice, rats and dogs after single and repeated administration of Compound 1. Pharmacokinetic parameters were estimated using noncompartmental analysis within WinNonlin v. 4.1. Quantification of Compound 2 was done by HPLC-MS/MS after extraction from plasma with acetonitrile. CD-1 mice, Sprague-Dawley rats, and beagle dogs were administered a single bolus intravenous injection of Compound 1 and blood sampled (terminal bleed, mouse, rat (exposure and gender data), n=3; serial bleed, rat and dog) between 5 min-24 hours. Bioavailability profile in mice was determined after administration of 50 mg/kg IV, IP, and PO with blood sampling 15 min-16 hr post administration. For rising dose experiments measuring exposure for each species, sets containing several animals were singly dosed at a given dose.

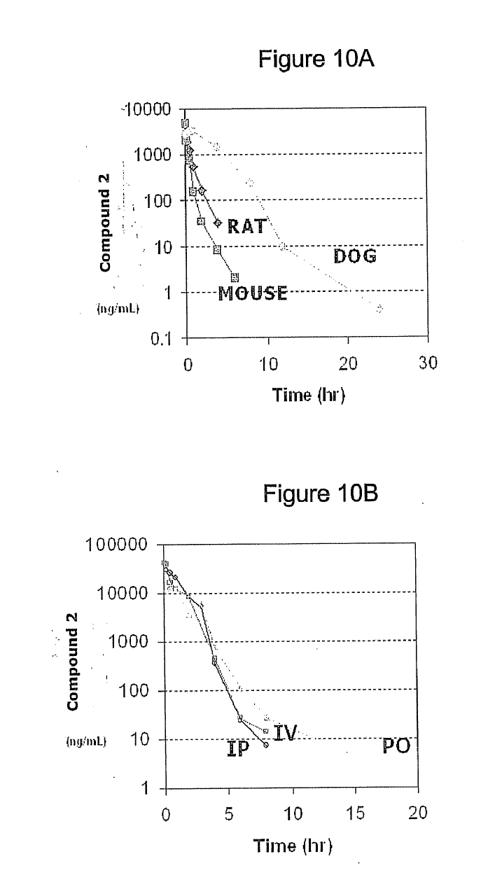

Results from single-dose experiments are shown in FIG. 10A, Table 43, and in FIG. 10B and Table 44, respectively. FIG. 10A shows a decrease in plasma concentration of Compound 2 over time in mouse, rat, and dog after a single intravenous dose. Pharmacokinetic parameters for the study are provided in Table 43. C₀ is initial concentration extrapolated to time zero. AUC_{INF} is area under the plasma-concentration time curve from time zero extrapolated to the infinite time. CL is clearance; Vₛₛ is steady state volume of distribution. T_{1/2} is half-life.

TABLE 43

| | MOUSE | RAT | DOG |
|---|---|---|---|
| Dose (mg/kg) | 5 | 5 | 5 |
| Dose (mg/m²) | 15 | 30 | 90 |
| C₀ (μg/mL) | 7.1 | 3.6 | 2.8 |
| AUC_{INF} (μg*hr/mL) | 1.8 | 2.1 | 12.3 |
| CL (mL/min/kg) | 47.6 | 41.2 | 6.8 |
| Vₛₛ (L/kg) | 1.0 | 1.8 | 1.0 |
| T_{1/2} (hr) | 1.4 | 0.8 | 1.1 |

In mice, a single dose of Compound 1 was administered intravenously, intraperitoneally, or orally, and decrease in plasma concentration of the compound over time is shown in FIG. 10B. Pharmacokinetic parameters for the study are provided in Table 44. Abbreviations are as in Table 18; F is fraction of dose absorbed. Compound 2 is rapidly and extensively distributed in both mice and rats when dosed IV, IP, or PO.

TABLE 44

| | IV | IP | PO |
|---|---|---|---|
| Dose (mg/kg) | 50 | 50 | 50 |
| C₀ (μg/mL) | 42.0 | — | — |
| AUC_{INF} (μg*hr/mL) | 43.6 | 46.2 | 28.3 |
| CL (mL/min/kg) | 19.1 | — | — |
| Vₛₛ (L/kg) | 1.2 | — | — |
| T_{1/2} (hr) | 0.8 | 0.7 | 3.3 |
| F (%) | — | 106 | 65 |

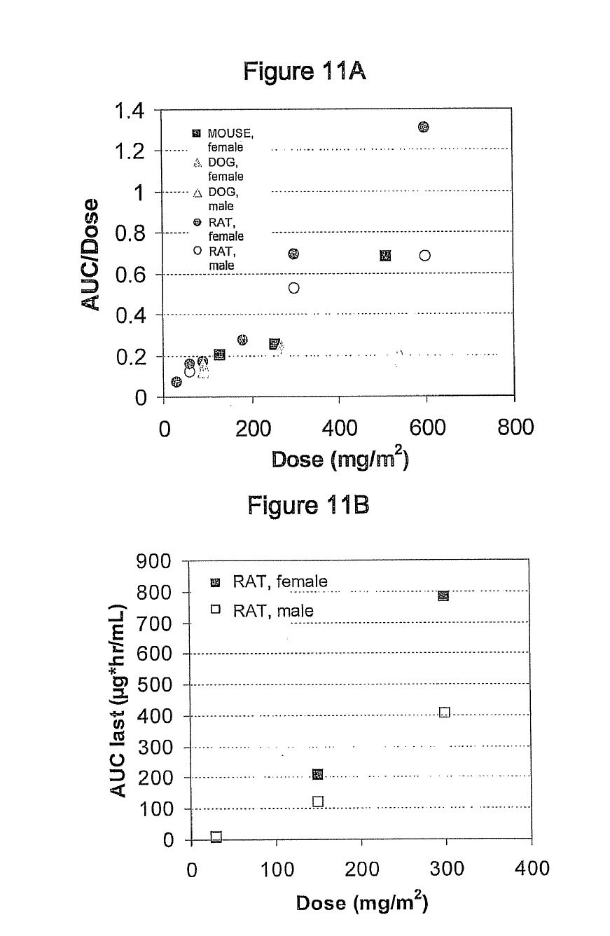

The results of rising dose pharmacokinetic studies are shown in FIG. 11 and in Table 45. In rising dose pharmacokinetic studies, Compound 2 displayed non-linear systemic exposure; the area under the concentration curve (AUC) increased more than dose linearly. As shown in FIG. 11A this non-linear systemic exposure was most pronounced in rats and mice and occurs to a lesser extent in dogs. As can be seen in Table 45, the non-linear PK observed in rat correlated with changes in clearance.

Gender-related differences in pharmacokinetic parameters were observed in rodents and to a much lesser extent in dogs. As shown in FIG. 11B, Female rats had 1.3- to 2-fold greater plasma AUC than male rats. “AUC last” on the plot corresponds to the area under the curve taken between the first and last measured time points.

TABLE 45

| | Dose | | |
|---|---|---|---|
| | 60 mg/m² | 300 mg/m² | 600 mg/m² |
| CL (mL/min/kg) | 17.2 | 4.0 | 2.0 |
| Vₛₛ (L/kg) | 1.3 | 1.1 | 1.1 |
| T_{1/2} (hr) | 1.4 | 1.7 | 5.9 |

### Example 34

### Mass Balance and Elimination

¹⁴C-Labeled Compound 1, with the label on the free base, was administered as a IV bolus dose of 50 mg/kg to male rats. Whole-body autoradiography indicated ¹⁴C-Compound 2-related radioactivity was widely distributed in tissues after an IV bolus dose with maximum concentrations observed 1 hour post dose.

Treated rats were further cannulated in femoral vein and the bile duct to allow for the evaluation of the rate and extent of elimination of total radioactivity from urine, bile, and feces. Total radioactivity was analyzed by liquid scintillation counting. Samples were also subject to HPLC-radiometric detection to elucidate the metabolic and elimination profile of Compound 2.

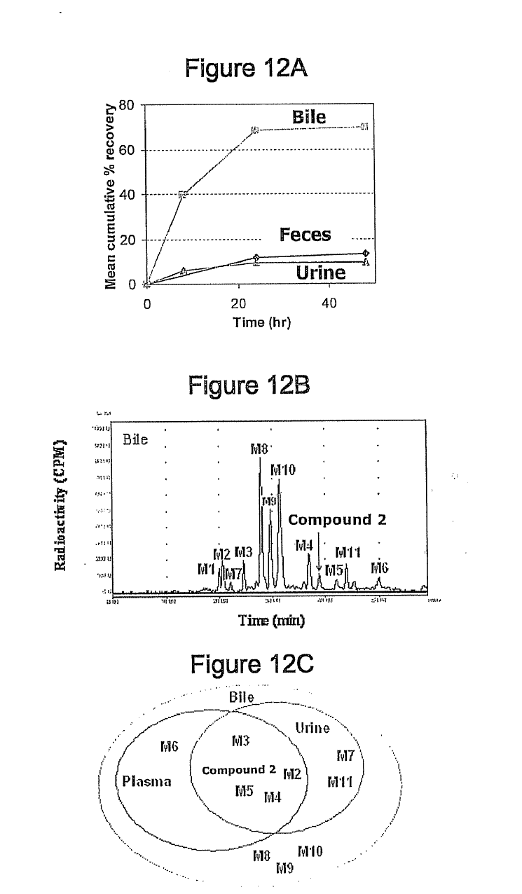

Results of elimination studies are shown in FIG. 12 and in Table 46. It can be seen from the FIG. 12A and Table 46 that Compound 2 is predominately eliminated in bile (i.e., by biliary excretion).

TABLE 46

| | | Mean cumulative |
|---|---|---|
| | Interval (hr) | % recovery |
| Feces | 0-24 | 11.8 |
| | 24-48 | 13.2 |
| Bile | 0-8 | 39.7 |
| | 8-24 | 68.5 |
| | 24-48 | 69.5 |
| Urine | 0-8 | 6.1 |
| | 8-24 | 9.4 |
| | 24-48 | 9.6 |
| Cumulative | 24 | 89.7 |
| Total | 48 | 92.3 |

FIG. 12B shows various metabolites (M1-M11) of Compound 2 as observed in rat bile by HPLC. Preparation of samples for metabolite analysis was as follows. Plasma samples were extracted by protein precipitation with acetonitrile. The extraction was preformed by adding ice cold acetonitrile (3 parts) to plasma (1 part v/v). After the samples were mixed using a benchtop vortex mixer the samples were centrifuged, the supernatants were transferred to silanized glass tubes, evaporated to dryness under nitrogen, and reconstituted in 50/50 acetonitrile/water solution. Urine samples were directly injected. Bile samples containing radioactivity were diluted in water prior to injection.

Metabolites in plasma, urine, and bile were separated on a reverse phase HPLC column with an Agilent 1000 system (Santa Clara, Calif.). Separation of Compound 2 and Compound 2-derived metabolites was achieved on a 250×4.6 mm 4 micron C18 Synergi Hydro column (Phenomonex, Torrance, Calif.) using mobile phase A of 0.1% formic acid in water and mobile phase B of acetonitrile. The flow rate was 0.75 mL/min with the following gradient: 0-2 min hold at 10% B followed by a linear gradient to 30% B at 45 min; 45-47 ramping to 90% B and held for 2 min; 49-50 min ramping from 90% to 10% B and held for 2 min; 52 to 55 min ramping to 90% B and back to 10% B at 57 min and held for the completion of the run. For ¹⁴C detection the HPLC was coupled to a Radiomatic 610TR Flow Scintillation Analyzer equipped with a 500 μL liquid cell (PerkinElmer Life Sciences, Waltham, Mass.) using a scintillation fluid flow rate of 2.25 mL/min.

Taken together, FIG. 12A, 12B, and Table 46 demonstrate that the majority of Compound 2 is eliminated as metabolized drug in rats. A graphical depiction of the location and distribution of the observed Compound 2 metabolites in rats, as mapped onto the circulating and elimination pathway, is provided in FIG. 12C.

### Example 35

### Protein Binding Studies

Studies were performed using the Rapid Equilibrium Dialysis (RED) device (Linden Bioscience). Inserts were soaked in water for 10 min×2, then removed and drained immediately prior to use. Inserts were placed into a PTFE base plate prior to the addition of spiked matrix (Compound 1 in plasma at 15 μM) and buffer. All experiments were performed in duplicate and each chamber was sampled in duplicate. The samples were incubated for 4-6 h at 37° C. in a rotating incubator (100 rpm). Compound 2 was quantified using LC-MS/MS. Data from duplicate samples each sampled twice.

It was determined that in each of mouse plasma, dog plasma, and human plasma, Compound 2 is highly protein-bound. At the concentration used, the mean percentage of Compound 2 that is protein-bound is greater than or equal to 99.9% for each of mouse plasma, dog plasma, and human plasma.

### Example 36

### Calculation of Combination Index

A combination index compares the concentration of compounds dosed in combination required for a given fractional effect to the concentration of single agent compound required to give the same fractional affect. In this application, the fractional effect is EC₅₀.

$${CI}_{50} = \frac{D 1 Combo {EC}_{50}}{{D}_{1} {EC}_{50}} + \frac{{D}_{2} Combo {EC}_{50}}{{D}_{2} {EC}_{50}}$$

The equation above represents the theoretical additive response for two mutually exclusive drugs, and takes into consideration the ratio at which the two compounds are dosed. When CI₅₀=1, then drugs are additive, as if using twice as much of either drug alone. When CI₅₀<1, less compound is required for a given fractional effect, and the combination is synergistic. When CI₅₀>1, more compound is required, and the combination is antagonistic. The process by which CI₅₀ values were determined in this application is described in the figures below which illustrate hypothetical outcomes for interactions of equipotent drugs (10 nM EC₅₀).

The following equations are examples of additive, antagonistic, and synergistic scenarios using the equation above, and where Drug 1 and Drug 2 are equipotent with an EC₅₀ of 10 nM.

$$\begin{matrix}{CI}_{50} = \frac{5}{10} + \frac{5}{10} = 1 & Additive \\ {CI}_{50} = \frac{10}{10} + \frac{10}{10} = 2 & Antagonistic \\ {CI}_{50} = \frac{1.5}{10} + \frac{1.5}{10} = 0.3 & Synergistic\end{matrix}$$

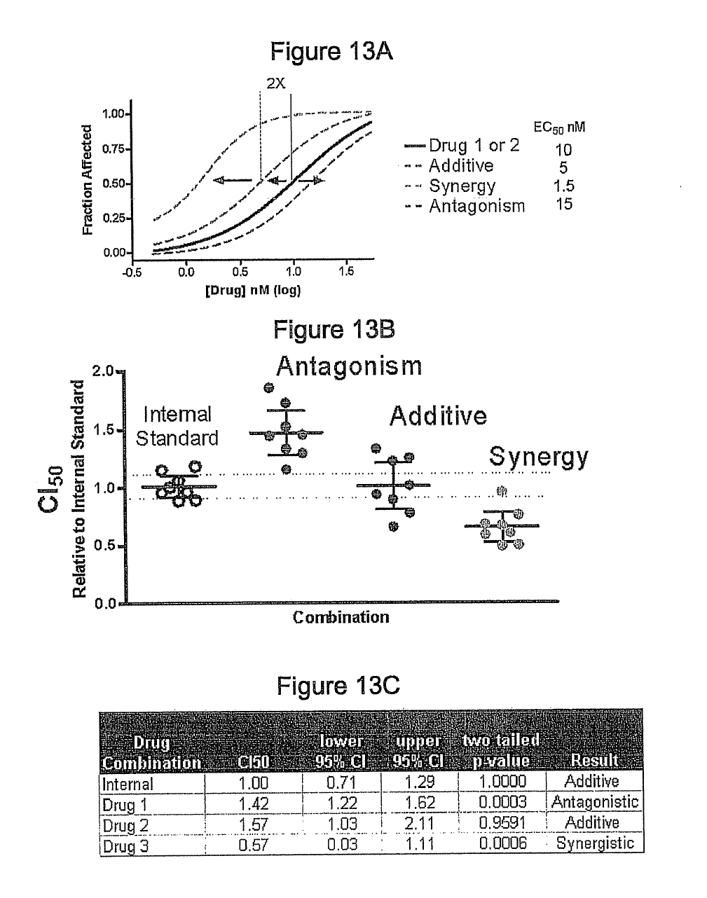

FIG. 13 shows an example of how interaction between two drugs can be determined by measuring corresponding dose-responses. FIG. 13A generically depicts the interpretation of EC₅₀ values for single agents and for combinations. FIG. 13B generically depicts calculation of CI₅₀ values for drug dosed with itself, or in combination with other drugs. Data from independent experiments may be plotted with 95% confidence intervals. FIG. 13C generically depicts results from the Mann-Whitney test that was used to calculate a p-value and determine statistical significance from the additive internal control.

### Example 37

### In Vitro Combination Studies

A colorectal carcinoma cell line, HCT 116 with either intact p53 (p53+/+) or suppressed p53 (p53−/−) protein levels, was treated in vitro with Compound 1 in combination with a panel of chemotherapeutic agents using either co-dosing or sequential dosing schedules, as described in further detail below. High content cell imaging and a cell proliferation assay were used to measure the anti-proliferative effects of the compounds.

HCT 116 cells transfected with p53 RNAi or a control vector were cultured in DMEM, 10% FBS, and 1× antibiotic/antimycotic. Cells were plated in growth medium in black/clear Falcon® 384-well plates. Cells were treated to assess the effects of p53 status, drug dose ratios, and dose schedules. A dilution series of Compound 1 combined with a dilution series of various cytotoxics: gemcitabine (Gem), 5-fluorouracil (5-FU), docetaxel (DTX), vincristine (VIN), carboplatin (Carbo), SN38, daunomycin (Dauno), cisplatin (Cis), nocodazole (NOC), or Compound 1 (internal additive control) was applied to cells. The three dose ratios tested were (Compound 1/Panel), high/high, low/high, and high/low, where the “high” compound dose response is generated starting at 10×EC₅₀ and “low” compound is 1×EC₅₀. Dose schedules were tested by combining compounds as a co-dose (i.e. simultaneous administration), or sequential washout dose starting with either Compound 1 or a panel compound. All procedures were performed by a Tecan robotic platform.

### Cell Count Assay

After overnight growth, cells were treated with compound for a total of 72 hours and incubated at 37° C., 5% CO₂. Cells were fixed in 4% formaldehyde and stained with 1:4000 dilution of 10 mg/mL Hoechst 33342. HCS images were captured and data analyzed using the Target Activation application, object count per field parameter, on the ArrayScan VTI instrument (Cellomics, Inc.).

### Proliferation Assay

Cells were plated and treated as described in the cell count assay with the exception of an extended incubation period of 6 days. A CellTiter Blue® cell viability assay (Promega) method was applied according to the manufacturer's instructions.

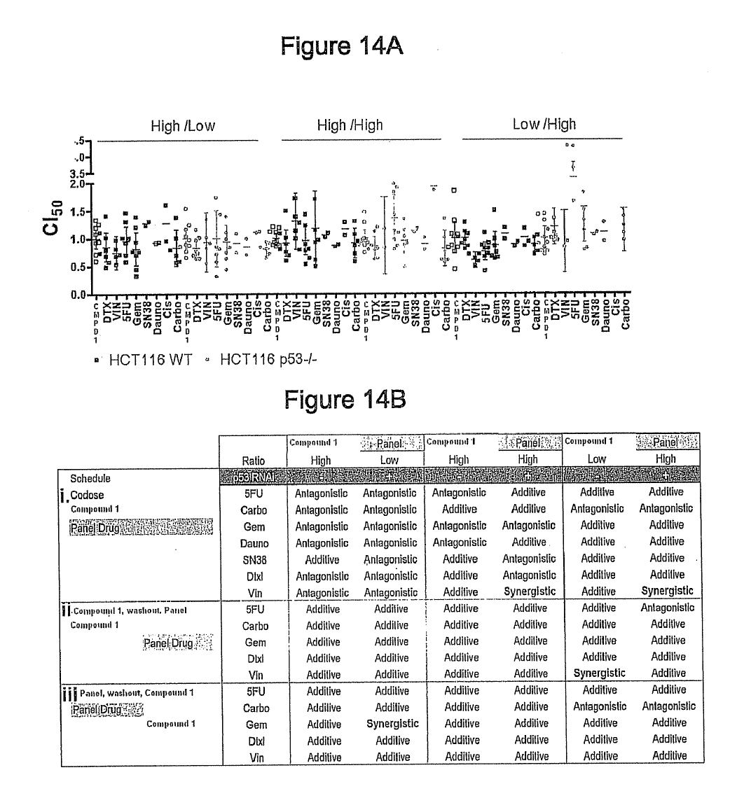

FIG. 14A and FIG. 14B shows results using the cell count assay for combination studies in HCT 116 cells conducted under three dosing ratios in the cell count assay. Studies were performed in p53+/+ and p53−/− (i.e. without and with p53 RNAi, respectively). It can be seen from the FIGS. 14A and 14B that conditional synergies were observed in vitro combined with gemcitabine (Gem), docetaxel (Dxtl), and vincristine (Vin). In other words, synergies with the second agent were dependent in certain cases on the ratios of compounds used or p53 status of the cells.

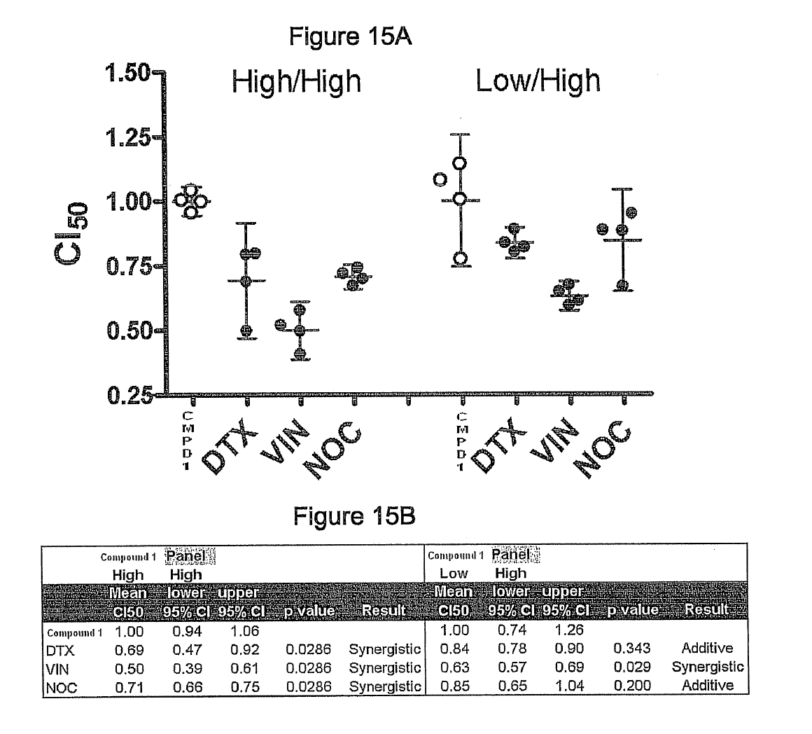

FIG. 15 shows results obtained using the proliferation assay, demonstrating that microtubule targeted agents (i.e. spindle toxins) show synergy in combination with Compound 1 under certain conditions. These microtubule-targeted agents target the mitotic spindle in dividing cells. The sequence of administration was Compound 1, washout, and then docetaxel (DTX), vincristine (VIN), or nocodazole (NOC). High/High ratios of Compound 1/panel drug are on the left and Low/High ratios of Compound 1/panel drug are on the right.

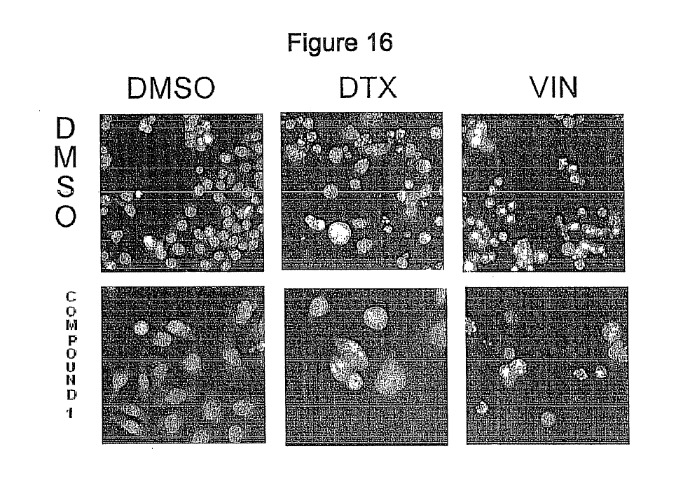

FIG. 16 shows HCS images of HCT 116 cells treated with Compound 1, docetaxel (DTX), or vincristine (VIN), alone, and Compound 1 in combination with docetaxel or with vincristine. As can be seen from the figure, in cells that were treated with compound 1 alone or in combination, polyploidy was observed. Certain treatments and combinations of treatments also led to chromatin condensation or fragmentation.

In general, the most profound anti-proliferative effects were observed with Compound 1 and agents that disrupt microtubule polymerization such as vincristine and nocodazole. Statistically significant synergy was observed in p53−/− HCT 116 cells when Compound 1 was co-dosed with high doses of vincristine. Sequential dosing of Compound 1 followed by each chemotherapeutic compound showed significant synergy with vincristine and nocodazole, a trend toward synergy with docetaxel (i.e., under certain conditions), and additive anti-proliferative effects with carboplatin, gemcitabine, 5-fluorouracil, daunomycin, and the active metabolite of irinotecan, SN38.

### Example 38

### In Vivo Combination Studies Using Compound 1 and Docetaxel

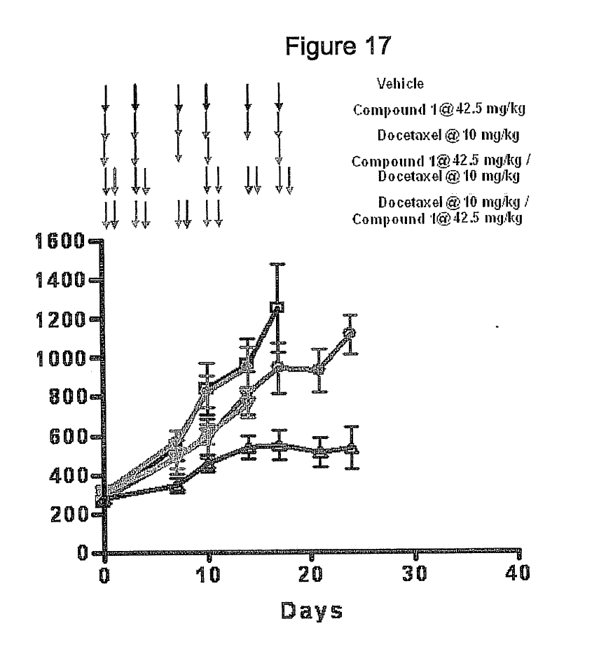

The in vivo anti-tumor activity of Compound 1 in combination with docetaxel (Taxotere®) was evaluated in female mice (nu/nu) subcutaneously implanted in the right hind flank region with 200 ml of a 2.5×10⁷ cells/mL suspension (1:1 DPBS with cells: Matrigel) of HCT 116 colorectal carcinoma cells. Treatments were initiated when tumors reached an average volume of 200 mm³; mice were randomized into groups and treated with vehicle, Compound 1, docetaxel or with either sequential combination of Compound 1 and docetaxel administered with 24 hours separation. Results are shown in FIG. 17.

End points for each group were determined based on body weight nadir, adverse clinical observations, or tumor volumes exceeding maximum threshold of 2000 mm³. Responses were assessed by tumor growth inhibition and tumor growth delay. TGI and TGD in the treatment group were evaluated against the vehicle control group.

Compound 1 was administered IP on day 0, 3, 7, 10, 14 and 17 at a dose of 42.5 mg/kg (shown as open circles, FIG. 17); docetaxel was administered IP on day 0, 3, 7, 10 and 17 at a dose of 10 mg/kg (shown as solid circles, FIG. 17). The sequence Compound 1→docetaxel was accomplished by the administration IP of Compound 1 on day 0, 3, 10, 14 and 17 and of docetaxel on day 1, 4, 11, 15 and 18 (shown as open triangles, FIG. 17). The sequence docetaxel→Compound 1 was accomplished by the IP administration of docetaxel on day 0, 3, 7 and 10 and of Compound 1 on day 1, 4, 8 and 11 (shown as open inverted triangles, FIG. 17).

### Example 39

Compound 1 was formulated as a sterile, clear, colorless-to-yellow liquid for intravenous (IV) infusion. The formulation contained 10 mg/mL Compound 2 (the free base of Compound 1), 200 mg/mL of sulfobutyl ether beta-cyclodextrin, sodium salt (e.g., Captisol®) as a solublizing excipient, hydrochloric acid for pH adjustment, and Water for Injection (qs). The formulation had a pH of 3.0. In certain embodiments, the formulation for injection has a pH of about 2.5 to 3.5. The formulation for injection was manufactured without preservatives under current Good Manufacturing Practice (GMP). In certain embodiments, the formulation has a total impurity content of less than about 3% by weight.

Compound 1 formulation for injection was supplied in 25 mL Type 1 glass vials. Each vial contained sufficient Compound 2, at a concentration of 10 mg/mL, to permit administration of 200 mg of Compound 2 to a patient. A 6% fill overage was included for vial-needle-syringe withdrawal loss. Each single-use vial was labeled individually. The formulation is packaged in cartons that may contain multiple vials per carton. The cardboard carton also provides protection from light.

Before IV administration, Compound 1 formulation was diluted with 5% Dextrose in Water, USP, (D5W) to concentrations between 0.5 mg/mL and 5.0 mg/mL, measured as free base concentrations. Once prepared, these dilutions were stable for up to 32 hours, when stored at ambient conditions.

### Example 40

Compound 1 formulation for injection was administered weekly for 3 consecutive weeks of a 28-day cycle. In one embodiment, Compound 1 formulation for injection was given as a 3-hour infusion. In one embodiment, Compound 1 formulation for injection was given on Day 1, Day 8 and Day 15 of the 28-day cycle.

Pharmacokinetic (PK) evaluation was performed on Days 1 and 15. PK analysis showed that Compound 2 declines with a terminal half-life of 7 hours and has a moderate to low clearance. Pharmacokinetic parameters (including plasma exposure) were similar after the first and third-weekly dose administrations, indicating no change in Compound 2 disposition following repeated administration of Compound 1. At all dose levels time vs. concentration profiles showed spikes in plasma concentrations or a flat terminal phase, which is suggestive of entero-hepatic recirculation of Compound 2.

### Example 41

The activity of Compound 1 was studied in the human cell line HCT 116 established as subcutaneous xenografts in nu/nu female mice. For each study, animals were randomized by tumor volume and distributed into groups of ten animals each. Treatments were initiated when tumor volume averaged about 200 mm³. Compound 1 was administered intraperitoneally (IP) biweekly for 3 weeks (BIW×3) at a dose of 150 mg/kg.

### Effects on Target Activity in Tumors and Normal Tissues

Inhibition of histone H3 (HH3) phosphorylation was evaluated in HCT 116 xenograft tumors, mouse femur bone marrow, and mouse skin punch biopsy sections by immunohistochemistry (IHC). HCT 116 xenograft tumors, femurs, skin punches were excised from mice treated biweekly for three weeks BIW×3 (on Days 1, 4, 8, 11, 15, and 18) with Compound 1 at a dose of 150 (skin) or 170 (bone marrow) mg/kg IP. The tumors were collected 6 hrs post-dose on day 4, 11, 18 and on day 25 (one week after completion of dosing phase of the experiment).

Phosphorylated histone H3 (pHH3) was detected by immunohistochemistry staining of tissue sections with the antibody # 9701 (Cell Signaling Technology, Inc.), which recognizes phosphorylation of Ser10 residue in histone H3 protein.

### Effects in Mouse Skin Punches

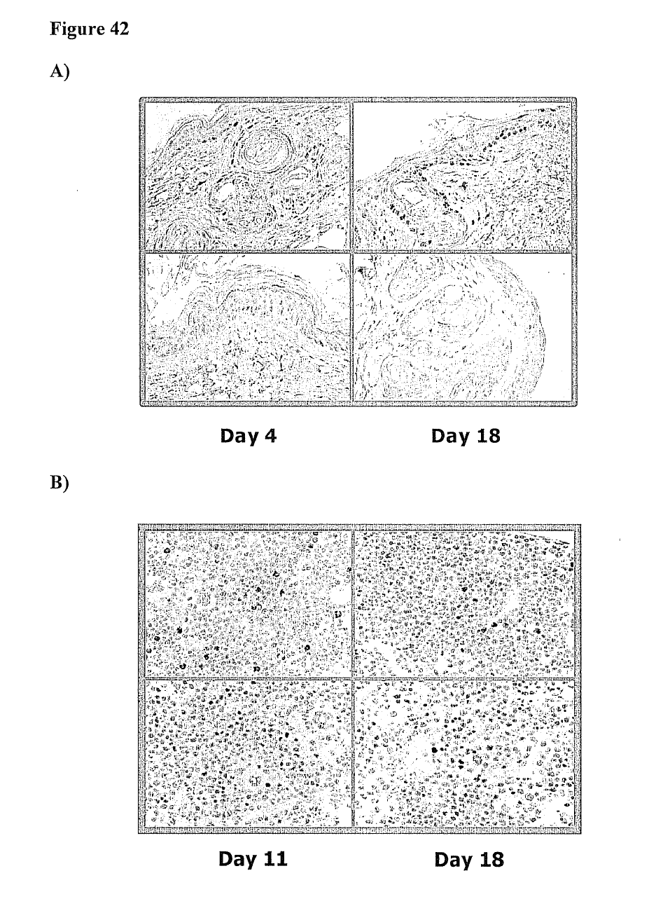

Photomicrographs of skin punches from nu/nu athymic mice after treatment with 150 mg/kg Compound 1 biweekly for 3 weeks. Three mice in each group were sacrificed on day 4 and day 18, 6 hours post-dose. Skin punches (8 mm) were fixed in formalin, trimmed, and sections stained to identify cells positive for histone H3 phosphorylation. The epidermis of mice exposed to Compound 1 displayed a decreased number of phospho-histone H3-positive cells; Compound 1 was able to reduce ˜50% the number of positively stained cells as compared with cells from vehicle-treated mice at day 4 and day 18 of the study (FIG. 42A). On day 25 of the experiment, the number of positively stained cells in the epidermis of Compound 1-treated mice was still decreased compared to vehicle-treated mice.

### Effects in Mouse Bone Marrow

Photomicrographs of sections of mouse femurs after treatment with 170 mg/kg of Compound 1 on a BIW×3 schedule show a significant drug-induced effects on histone H3 phosphorylation (FIG. 42B). Bone marrow cells positive for this staining were 3 and 7 times less evident at day 11 and day 18, respectively, after treatment with 170 mg/kg Compound 1 IP as compared with vehicle treatment. Histone H3 phosphorylation had recovered to normal levels at day 25.

### Example 42

Clinical pharmacodynamic assessments were performed as follows. Skin punch biopsy samples were collected prior to (e.g., just prior to or up to 14 days prior to) treatment and during Cycle 1, Day 1 of treatment at between 3 and 7 hours after the start of the 3-hour infusion. Skin punch biopsy samples were analyzed for inhibition of histone H3 phosphorylation. Based on average in vitro cellular pHH3 EC₉₀ estimates, the target serum concentration is 1 μM, which was achieved at all dose levels for a minimum duration of 4 hours. The duration for which the estimated target serum concentration threshold was achieved is provided in Table 47.

TABLE 47

| Dose | Duration of target serum |
|---|---|
| (mg/m²) | concentration (hours) |
| 30 | 4 |
| 60 | 8 |
| 120 | 10 |
| 240 | 20 |
| 480 | 20 |

Inhibition of pHH3 induced by administration of Compound 1 was observed in skin biopsies of patients treated at doses of 240 mg/m² and greater. At the 240 mg/m² dose level, serum Compound 2 levels exceeded the preclinical target inhibitory levels.

In addition to inhibition of phosphorylation of HH3, skin punch biopsies can also be tested for the appearance of polyploidy.

Patients with readily accessible tumors (such as skin, nodal, or liver metastases) undergo tumor biopsies. Tumor biopsy samples are obtained prior to treatment and on cycle 1, Day 22. Optionally, additional biopsies are also obtained. Tumor biopsy samples are analyzed for appearance of polyploidy and other markers of apoptosis or cell cycle changes.

In addition to skin punch and tumor biopsy samples, historic (e.g., paraffin-embedded pretreatment) samples, if available, are analyzed for baseline expression of proliferation and other markers of apoptosis or cell cycle changes. Samples may be assessed as shown in Table 48.

TABLE 48
Pharmacodynamic Assessments

| Markers | Tissue Samples |
|---|---|
| pHH3 | skin, tumor |
| Polyploidy | skin, tumor |
| Ki67 | Tumor |
| Caspase | Tumor |
| Retinoblastoma (Rb) | Tumor |
| p53 | Tumor |
| p21 | Tumor |
| BCRA1 | Tumor |
| Aurora A | Tumor |

### Example 43

Mice received 200 μL of a 5×10⁶ HCT 116 colorectal cancer cell suspension (1:1 Dulbecco's phosphate-buffered saline with cells:Matrigel) as a subcutaneous injection in the right hind flank. When tumors reached an average volume of 400 mm³, mice were sorted into randomized groups of 3 per time point. For the dose escalation arm; mice were administered 1, 2, 5, 10, or 20 mg/kg Compound 1 IP. At 1 hr postdose tumor and plasma was collected and snap-frozen in liquid nitrogen and stored frozen at −80° C. until samples were processed for analysis. For the time-course arm, mice were administered an IP injection of 170 mg/kg Compound 1 followed by collection of plasma and tumor 6, 9, and 24 hr post-dose.

### Western Blot Assay

Tumor samples were frozen on liquid nitrogen, and ground into a fine powder. Lysis buffer containing phosphatase inhibitors was added to the tumor powder before homogenization and a snap freeze cycle. The cellular debris was removed by centrifugation, and the protein concentration was measured using the BioRad DC Protein Assay. Twenty-five (25 μg) of protein was loaded on NuPAGE 4-12% Bis-Tris Gel and separated by electrophoresis at a constant 200V. Protein was transferred to PVDF membrane at a constant 30 V for 1 hr using the Invitrogen XCell II Blot Module transfer system and, upon completion, the membranes were incubated with 5% milk in TBST (Tris-buffered saline with Tween) at room temperature for 1 hr. The membranes were incubated with antibody against pHH3 or total HH3 (#9701 and #9715, respectively, Cell Signaling Technology) in TBST, overnight at 4° C. Membranes were washed in TBST, and then incubated with anti-rabbit IgG-HRP (#NA934V, GE HealthCare) in TBST for 1 hr at room temperature. Membranes were washed with TBST, and antibodies were detected with ECL Plus chemiluminescent detection system (Amersham), followed by exposure to Kodak BioMax film.

### Western Blot Analysis

Films were visually assessed for total histone H3 (HH3) and histone H3 phosphorylation (pHH3). Total HH3 levels served as loading controls. Tumor pHH3 levels from mice treated with Compound 1 were compared to samples obtained from vehicle control mice.

### ELISA Analysis

Phospho-histone H3 levels were determined using a commercial ELISA kit from (# KHO0671, Biosource/Invitrogen). The conditions were as described by the manufacturer with 100 μg total proteins from tumor lysates prepared as described in this Example.

Preparation of Plasma Samples. Plasma Samples were Extracted by Protein precipitation with acetonitrile. The extraction was preformed by adding 3 parts ice cold acetonitrile containing internal standard (verapamil) to 1 part plasma (v/v). After the samples were mixed using a benchtop vortex mixer the samples were centrifuged, the supernatants were transferred and diluted with water prior to analysis of Compound 2 levels.

HPLC-MS/MS. Compound 2 in plasma was separated on a reverse phase HPLC column with an Agilent 1000 system (Santa Clara, Calif.). Chromatography achieved on a 30×2 mm 4 μm C18 Synergi Hydro-RP column (Phenomenex, Torrance, Calif.) using mobile phase A of 0.1% formic acid in water, and mobile phase B, acetonitrile. The flow rate was 0.70 mL/min with the following gradient: linear gradient between 0-3.5 min starting at 95% A and ending at 60% A, followed by step to 5% A at 3.6 min and held until 4.49 min; the gradient was stepped to 95% A at 4.5 min and served as a wash cycle for the column. This wash cycle was repeated between 4.5 and 5.5 min, at which time the starting conditions were restored and the column allowed to equilibrate for 30 seconds prior to the next run. The detector consisted of an API4000 (Sciex/ABI, Foster City, Calif.) triple quadrupole mass spectrometer using positive mode turbo electrospray ionization.

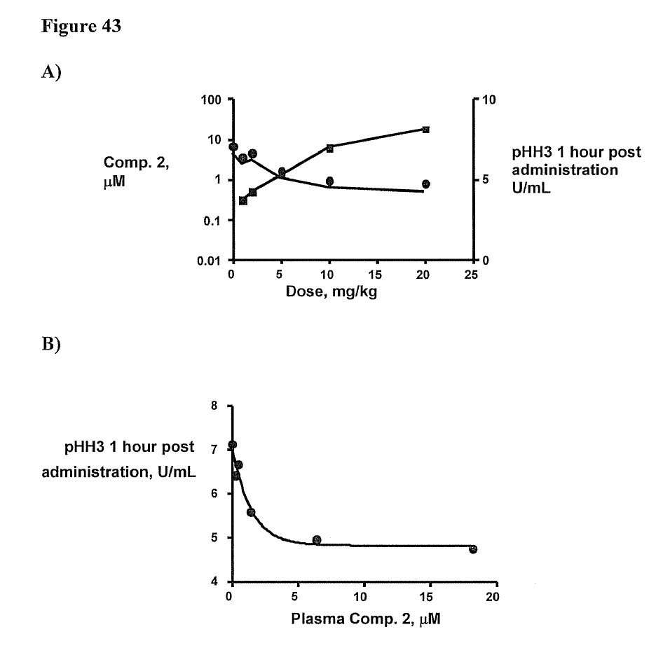

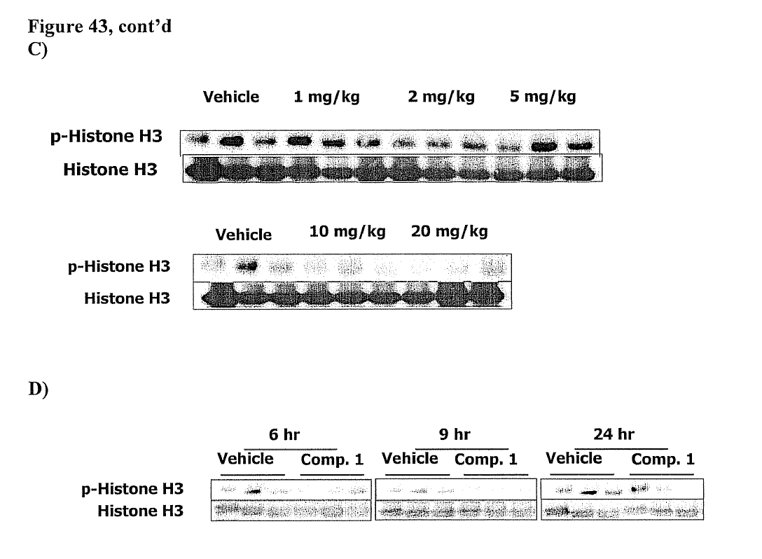

As can be seen in FIG. 43, increasing plasma concentrations of Compound 2 correlated with inhibition of phosphorylation of Histone H3 in tumor. FIGS. 43A and C show that low doses of Compound 1 administration modulated Histone H3 phosphorylation. FIG. 43B demonstrates that 5 μM plasma concentration of Compound 2 produced maximal inhibition of phosphorylation of Histone H3. FIG. 43D shows that at a single dose of 170 mg/kg Compound 1, maximal inhibition of phospho-histone H3 in tumor was maintained for up to 24 hours.

### Example 44

PARP cleavage was measured in HCT 116 (colon carcinoma) and MV-4-11 tumor lysates by western blotting. Lysates were made from xenograft tumors excised from mice treated with a single dose of Compound 1 at a dose of 170 mg/kg IP for HCT 116 and 50 or 100 mg/kg IP for MV4-11. HCT 116 tumors were collected 3, 6 and 12 hrs post dosing; MV-4-11 tumors were collected at 2, 6 and 24 hrs post dosing. Time-dependent effects of Compound 1 on the expression levels of the indicated protein were measured.

Tumors were lysed in cell extraction buffer (Biosource # FNN0011) containing protease inhibitors (Sigma # P2714), and PMSF Phenylmethanesulfonyl fluoride (PMSF) [#P7626, Sigma]. Forty micrograms of protein for each sample was loaded and run on 4-12% Tris-Glycine NuPAGE gel (Invitrogen), in Novex Tris-Glycine running buffer (Invitrogen). After gel separation, proteins were electro-transferred to a PVDF membrane (Invitrogen). Proteins were detected by incubating membranes in primary and secondary antibodies as indicated in Tables 49 and 50 below.

TABLE 49
Primary antibodies

| | | Buffer and |
|---|---|---|
| Name and vendor (product #) | Dilution/Host | incubation time |
| Cl.PARP (Cell Signaling | 1:1,000 rabbit | 5% milk/TBST o/n @ |
| Technology #9541S) | | 4° |
| Actin (Abcam # Ab6276) | 1:10,000 mouse | 5% milk/TBST for 1 |
| | | hr RT |

TABLE 50
Secondary antibodies

| Name and vendor (product #) | Dilution | Buffer and incubation time |
|---|---|---|
| Anti-rabbit (Amersham | 1:5,000 | 5% milk/TBST for 1 hr RT |
| #NA934V) | | |
| Anti-mouse (Amersham | 1:2,000 | 5% milk/TBST for 1 hr RT |
| #NA931V) | | |
| ECL detection reagent | Amersham | |

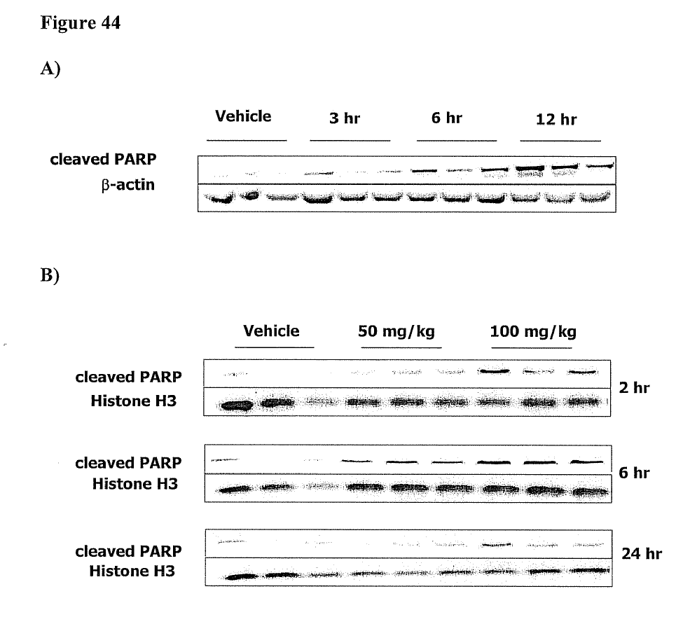

FIG. 44A shows that in HCT 116 tumor bearing mice treated with a single IP dose of 170 mg/kg Compound 1, that PARP cleavage became evident 3 hr after the dose, and is maintained for at least 12 hr after the dose. FIG. 44B shows that in MV-4-11 tumor bearing mice treated with a single IP dose (50 mg/kg or 100 mg/kg) of Compound 1, PARP cleavage was dose- and time-dependent.

While we have presented a number of embodiments of this invention, it is apparent that our basic teaching can be altered to provide other embodiments which utilize the compounds and methods of this invention. Therefore, it will be appreciated that the scope of this invention is to be defined by the appended claims rather than by the specific embodiments which have been represented by way of example.

1. Compound 1:

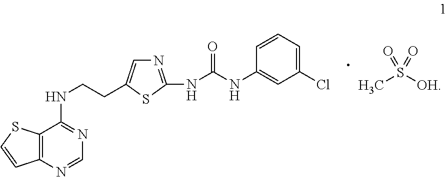

2. A crystalline form of the compound of claim 1.

3. The form according to claim 2, wherein said form is anhydrous and nonsolvated.

4. The form according to claim 2, wherein said form is a hydrate.

5. The form according to claim 2, wherein said form is a solvate.

6. The form according to claim 2, wherein said form is anhydrous.

7. The form according to claim 3, wherein said form is Form A of Compound 1.

8. The form according to claim 7, characterized in that it has one or more peaks in its XRPD pattern selected from those at about 8.5, 13.2, 15.3, 15.6, 16.7, 20.2, 20.6, 25.2, 26.4 and 27.0 degrees 2-theta and a differential scanning calorimetry pattern substantially similar to that depicted in FIG. 19.

9. The form according to claim 7, characterized in that the form has an X-ray diffraction pattern substantially similar to that depicted FIG. 18.

10. The form according to claim 4, wherein said form is Form B of Compound 1.

11. The form according to claim 10, characterized in that it has one or more peaks in its XRPD pattern selected from those at about 7.1, 10.5, 11.8, 17.0, 17.4, 18.0, 21.3, 23.7, 25.1, 25.8, 26.8, 27.4, and 27.7 degrees 2-theta and a differential scanning calorimetry pattern substantially similar to that depicted in FIG. 21.

12. The form according to claim 10, characterized in that the form has an X-ray diffraction pattern substantially similar to that depicted in FIG. 20.

13. A composition comprising Form A of Compound 1 and at least one other solid form of Compound 1.

14. The composition according to claim 13, wherein the other solid form is at least Form B of Compound 1.

15. A composition comprising the compound according to claim 1, and one or more compounds selected from:

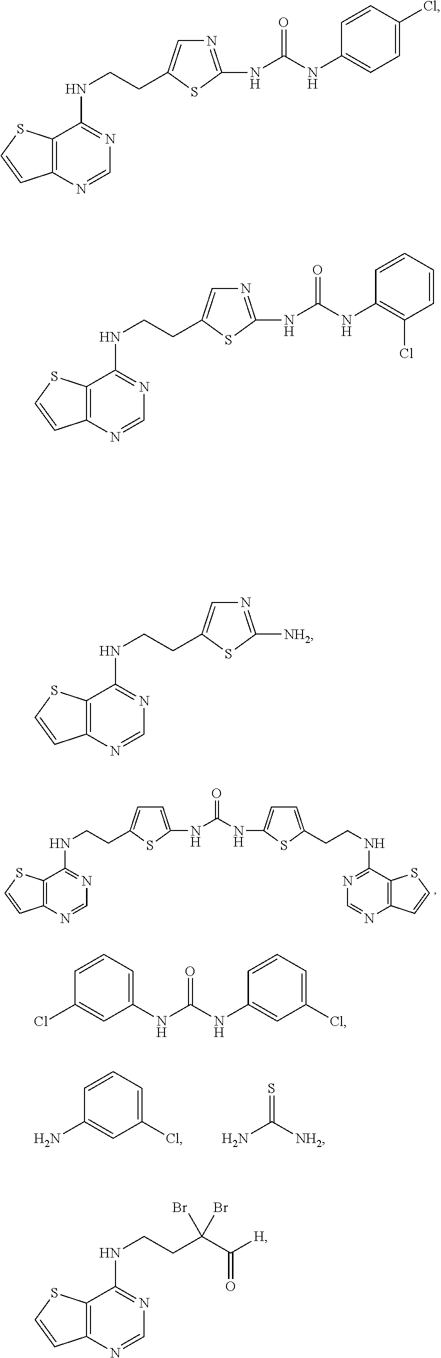

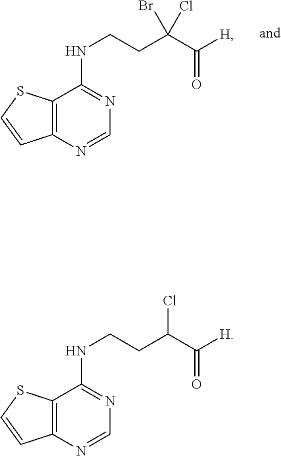

16. A composition comprising the compound according to claim 1 and a carrier.

17. A composition comprising the compound according to claim 1, and one or more pharmaceutically acceptable carriers, diluents, or excipients.

18. The composition according to claim 17, formulated for parenteral administration.

19. The composition according to claim 18, formulated for intravenous administration.

20. The composition according to claim 19, further comprising a solubility enhancer.

21. The composition according to claim 20, wherein the solubility enhancer comprises a cyclodextrin.

22. A method for treating cancer in a patient, comprising administering to the patient the composition according to claim 17.

23. The method according to claim 22, wherein the patient has a cancer characterized by a solid tumor or a hematological tumor.

24. The method according to claim 23, wherein the solid tumor cancer is selected from cancers of the colon, lung, prostate, ovary, breast, cervix, and skin.

25. The method according to claim 23 wherein the hematological tumor is a lymphoma or leukemia.

26. The method according to claim 25 wherein the lymphoma or leukemia is mantle cell lymphoma (MCL), Non-Hodgkin's lymphoma (NHL), Hodgkin's disease, acute myelogenous leukemia (AML), chronic myelogenous leukemia (CML), chronic lymphocytic leukemia (CLL) or acute lymphoblastic lymphoma (ALL).

27. A method for treating cancer in a patient, comprising administering the composition according to claim 17 to a patient having a cancer selected from bladder cancer, brain cancer, breast cancer, cervical cancer, colon cancer, esophageal cancer, head and neck cancer, leukemia, liver cancer, lung cancer, lymphoma, melanoma, myeloma, neuroendocrine cancer, ovarian cancer, pancreatic cancer, prostate cancer, renal cancer, sarcoma, skin cancer, stomach cancer, testicular cancer, thyroid cancer, and uterine cancer.

28. A method for treating cancer in a patient, comprising administering to a patient having a cancer an effective amount of a compound according to claim 1.

29. The method according to claim 28, wherein the compound is administered in a dose of about 30 mg/m²-2000 mg/m².

30. The method of claim 29 wherein the dose is administered once a week.

31. The method of claim 30 wherein the dose is administered once a week for three weeks.

32. The method of claim 30 wherein the dose administered is about 240 mg/m²-2000 mg/m².

33. The method of claim 30 wherein the dose administered is about 480 mg/m²-1800 mg/m².

34. The method of claim 33 wherein the dose administered is about 480 mg/m²-1500 mg/m².

35. The method of claim 33 wherein the dose administered is about 480 mg/m²-1200 mg/m².

36. The method of claim 33 wherein the dose administered is about 750 mg/m²-1500 mg/m².

37. The method of claim 33 wherein the dose administered is about 960 mg/m²-1200 mg/m².

38. The method of claim 28, further comprising administering a dose of a second active agent.

39. The method of claim 28, wherein the composition is administered prior to the dose of the second active agent.

40. The method of claim 38, wherein the second active agent is a spindle poison.

41. The method of claim 38, wherein the second active agent is selected from docetaxel, gemcitabine, vincristine, nocodazole, carboplatin, 5-fluorouracil, daunomycin, cisplatin and SN38.

42. A method for preparing Compound 2: 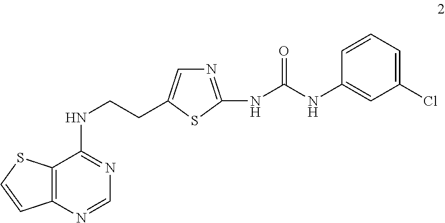
comprising the steps of:
(a) coupling INT1: 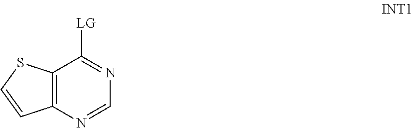
wherein LG is a suitable leaving group, with 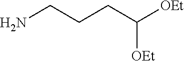
to form INT2: 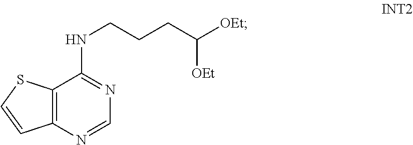
(b) deprotecting INT2 to form INT3; 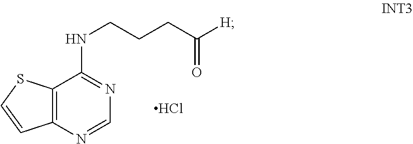
(c) brominating INT3 to form INT4: 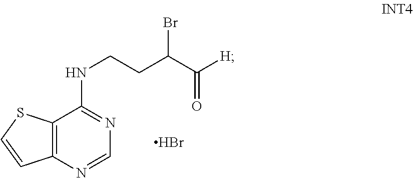
(d) coupling INT4 with thiourea to form INT5: 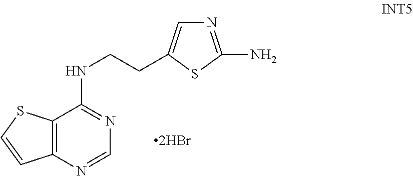
and (e) coupling INT5: 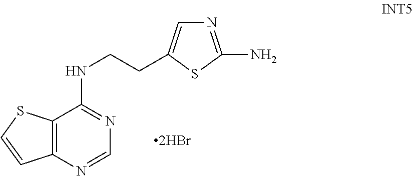
to 3-chlorophenyl-isocyanate to form Compound 2.

43. The method according to claim 42, further comprising the step of treating Compound 2 with methanesulfonic acid to form Compound 1: [chemical structure]
